SEQUENCE LISTING (78 sequences)

SEQ ID NO: 1 - protein, 8 aa, synthetic construct
GFKFSDHW

SEQ ID NO: 2 - protein, 8 aa, synthetic construct
ISEAGREK

SEQ ID NO: 3 - protein, 8 aa, synthetic construct
ISEDGREK

SEQ ID NO: 4 - protein, 14 aa, synthetic construct
ARVSTRVVNAGFDI

SEQ ID NO: 5 - protein, 14 aa, synthetic construct
ARVSTRVVNDGFDI

SEQ ID NO: 6 - protein, 6 aa, synthetic construct
NIADKN

SEQ ID NO: 7 - skipped

SEQ ID NO: 8 - protein, 10 aa, synthetic construct
QVWDSRSEYV

SEQ ID NO: 9 - protein, 121 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSL

SEQ ID NO: 10 - protein, 121 aa, synthetic construct
EVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSL

SEQ ID NO: 11 - protein, 107 aa, synthetic construct
SYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVL

SEQ ID NO: 12 - protein, 19 aa, synthetic construct
GGGSGGGGSGGGGSGGGGS

SEQ ID NO: 13 - protein, 247 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLGGGSGGGGSGGGGSGGGGSSYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVL

SEQ ID NO: 14 - protein, 247 aa, synthetic construct
SYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVLGGGSGGGGSGGGGSGGGGSEVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSL

SEQ ID NO: 15 - protein, 447 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 448 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 213 aa, synthetic construct
SYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 8 aa, synthetic construct
GFNFDDYT

SEQ ID NO: 19 - protein, 8 aa, synthetic construct
IRYNSVRI

SEQ ID NO: 20 - protein, 11 aa, synthetic construct
VKDEGFRSFEI

SEQ ID NO: 21 - protein, 11 aa, synthetic construct
VKDNGFRSFEI

SEQ ID NO: 22 - protein, 11 aa, synthetic construct
QSLEHSNGYHY

SEQ ID NO: 23 - skipped

SEQ ID NO: 24 - protein, 8 aa, synthetic construct
MQALQTWT

SEQ ID NO: 25 - protein, 118 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSS

SEQ ID NO: 26 - protein, 118 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSS

SEQ ID NO: 27 - protein, 111 aa, synthetic construct
DIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 28 - protein, 111 aa, synthetic construct
EIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 29 - protein, 19 aa, synthetic construct
GGGSGGGGSGGGGSGGGGS

SEQ ID NO: 30 - protein, 248 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSGGGSGGGGSGGGGSGGGGSDIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 31 - protein, 248 aa, synthetic construct
DIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIKGGGSGGGGSGGGGSGGGGSEVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSS

SEQ ID NO: 32 - protein, 444 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 445 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 218 aa, synthetic construct
DIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 8 aa, synthetic construct
GFTFSYHA

SEQ ID NO: 36 - protein, 8 aa, synthetic construct
ISDAGDRR

SEQ ID NO: 37 - protein, 8 aa, synthetic construct
ISDNGNRR

SEQ ID NO: 38 - protein, 17 aa, synthetic construct
ARTMDFDSGSHYYGLDV

SEQ ID NO: 39 - protein, 7 aa, synthetic construct
QTINSNY

SEQ ID NO: 40 - skipped

SEQ ID NO: 41 - protein, 10 aa, synthetic construct
QHYSRSPPYS

SEQ ID NO: 42 - protein, 124 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSS

SEQ ID NO: 43 - protein, 124 aa, synthetic construct
QEQLVQSGGGVVQPGRSLTVSCAGSGFTFSYHALHWVRQAPGKGLEWLAVISDNGNRRDYVDSVRGRFSVSRDNSRNTVFLQMDSLRADDSAVYFCARTMDFDSGSHYYGLDVWGQGTTVIVSS

SEQ ID NO: 44 - protein, 109 aa, synthetic construct
EVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVK

SEQ ID NO: 45 - protein, 19 aa, synthetic construct
GGGSGGGGSGGGGSGGGGS

SEQ ID NO: 46 - protein, 252 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSGGGSGGGGSGGGGSGGGGSEVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVK

SEQ ID NO: 47 - protein, 252 aa, synthetic construct
EVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVKGGGSGGGGSGGGGSGGGGSEVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSS

SEQ ID NO: 48 - protein, 450 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 451 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 216 aa, synthetic construct
EVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 10 aa, synthetic construct
GGGGSGGGGS

SEQ ID NO: 52 - protein, 218 aa, synthetic construct; identical to SEQ ID NO: 34

SEQ ID NO: 53 - protein, 702 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLGGGSGGGGSGGGGSGGGGSSYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVL

SEQ ID NO: 54 - protein, 213 aa, synthetic construct; identical to SEQ ID NO: 17

SEQ ID NO: 55 - protein, 706 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSGGGSGGGGSGGGGSGGGGSDIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 56 - protein, 213 aa, synthetic construct; identical to SEQ ID NO: 17

SEQ ID NO: 57 - protein, 710 aa, synthetic construct
EVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSGGGSGGGGSGGGGSGGGGSEVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVK

SEQ ID NO: 58 - protein, 216 aa, synthetic construct; identical to SEQ ID NO: 50

SEQ ID NO: 59 - protein, 708 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVALVESGGGLVQPGGSLRLSCTASGFKFSDHWMTWVRQAPGKGLEWVASISEAGREKVYVDSVRGRFTLSRDNAKKSLSLQMTSLRVDDTAKYHCARVSTRVVNAGFDIWGQGAMVIVSLGGGSGGGGSGGGGSGGGGSSYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVL

SEQ ID NO: 60 - protein, 218 aa, synthetic construct; identical to SEQ ID NO: 34

SEQ ID NO: 61 - protein, 707 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSGGGSGGGGSGGGGSGGGGSEVVLTQSPALLSLSPGERASLSCRASQTINSNYLAWYQLKPGQAPRLVIYLTSTRAPGIPARFSGSGSGTDFTLTIDRLEPEDFALYYCQHYSRSPPYSFGQGTRLDVK

SEQ ID NO: 62 - protein, 216 aa, synthetic construct; identical to SEQ ID NO: 50

SEQ ID NO: 63 - protein, 709 aa, synthetic construct
EVQLVQSGGGVVQPGRSLTVSCAGSGFTFSYHALHWVRQAPGKGLEWLAVISDAGDRRDYVDSVRGRFSVSRDNSRNTVFLQMDSLRADDSAVYFCARTMDFDSGSHYYGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDEGFRSFEIWGQGTMVIVSSGGGSGGGGSGGGGSGGGGSDIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 64 - protein, 218 aa, synthetic construct
EIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 702 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSLGGGSGGGGSGGGGSGGGGSSYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVL

SEQ ID NO: 66 - protein, 218 aa, synthetic construct; identical to SEQ ID NO: 64

SEQ ID NO: 67 - protein, 702 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSSYVLTQPPSVSAAPGETARITCGGKNIADKNVHWYQHRPGQAPVMVIYDDDDRPSEIPDRFSGSNSGNTATLTISRVEAGDEADYYCQVWDSRSEYVFGTGTTVTVLGGGSGGGGSGGGGSGGGGSEVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSL

SEQ ID NO: 68 - protein, 213 aa, synthetic construct; identical to SEQ ID NO: 17

SEQ ID NO: 69 - protein, 706 aa, synthetic construct
EVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSSGGGSGGGGSGGGGSGGGGSEIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIK

SEQ ID NO: 70 - protein, 213 aa, synthetic construct; identical to SEQ ID NO: 17

SEQ ID NO: 71 - protein, 706 aa, synthetic construct
EVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSEIVMTQSPLSLPVTPGEPASISCRSTQSLEHSNGYHYLDWYVQKAGQSPQLLISLGSKRASGVPDRFSGSVSGTDFTLKISRVEAEDVGVYYCMQALQTWTFGQGTKVEIKGGGSGGGGSGGGGSGGGGSEVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSS

SEQ ID NO: 72 - protein, 448 aa, synthetic construct
EVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSLASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 451 aa, synthetic construct
EVALVESGGGLVQPGGSLRLSCTASGFKFSDHWMTWVRQAPGKGLEWVASISEDGREKVYVDSVRGRFTLSRDNAKKSLSLQMTSLRVDDTAKYHCARVSTRVVNDGFDIWGQGAMVIVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 445 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 448 aa, synthetic construct
EVQLVESGGGVVQPGRSLTLTCVGSGFNFDDYTMHWVRQRPGKGLEWVSSIRYNSVRIEYVESVKGRFTTSRDNAKNSLYMEMKSLRPEDTAFYYCVKDNGFRSFEIWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 218 aa, synthetic construct; identical to SEQ ID NO: 64

SEQ ID NO: 77 - protein, 451 aa, synthetic construct
QEQLVQSGGGVVQPGRSLTVSCAGSGFTFSYHALHWVRQAPGKGLEWLAVISDNGNRRDYVDSVRGRFSVSRDNSRNTVFLQMDSLRADDSAVYFCARTMDFDSGSHYYGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 454 aa, synthetic construct
QEQLVQSGGGVVQPGRSLTVSCAGSGFTFSYHALHWVRQAPGKGLEWLAVISDNGNRRDYVDSVRGRFSVSRDNSRNTVFLQMDSLRADDSAVYFCARTMDFDSGSHYYGLDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0263472 A1
(43) Pub. Date: Aug. 21, 2025

(54) COMPOSITIONS AND METHODS FOR TREATING AND SUPPRESSING ALLERGIC RESPONSES

(71) Applicant: IgGenix, Inc., South San Francisco, CA (US)

(72) Inventors: DEREK CROOTE, SOUTH SAN FRANCISCO, CA (US); HENRY LOWMAN, EL GRANADA, CA (US)

(21) Appl. No.: 19/198,357

(22) Filed: May 5, 2025

Related U.S. Application Data
(63) Continuation of application No. PCT/US23/78898, filed on Nov. 7, 2023.
(60) Provisional application No. 63/382,963, filed on Nov. 9, 2022.

(51) Int. Cl. C07K 16/16; A61K 39/00; A61P 37/08

(52) U.S. Cl. CPC C07K 16/16; A61P 37/08; A61K 39/00; A61K 2039/505; A61K 2039/577; C07K 2317/31; C07K 2317/35; C07K 2317/53; C07K 2317/565; C07K 2317/622; C07K 2317/92

(57) ABSTRACT

Disclosed herein are antibodies, compositions, and methods of treating allergic responses. In some embodiments, the compositions are combination treatments of more than one antibody. In some embodiments, the antibodies bind to Ara h 2 and/or Ara h 6 and are useful for treating peanut allergies.

### CROSS-REFERENCE TO RELATED APPLICATION

The present application is a continuation of International Patent Application No. PCT/US23/78898, filed Nov. 7, 2023, which claims the benefit of U.S. Provisional Application No. 63/382,963 filed Nov. 9, 2022, which is incorporated herein by reference in its entirety.

### DESCRIPTION OF THE XML FILE SUBMITTED ELECTRONICALLY

The instant application contains a Sequence Listing which has been submitted electronically in XML format and is hereby incorporated by reference in its entirety. Said XML copy, created on Apr. 30, 2025, is named 56998-711_301SL.xml and is 84,276 bytes in size.

### SUMMARY

Disclosed herein are compositions that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10. In some embodiments, the VL of the first binding domain comprises an amino acid sequence with at least 80%, 85%, 90%, 95%, 98%, 99%, or 100% sequence identity to SEQ ID NO: 11. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11. In some embodiments, the first binding domain comprises a Fab, Fab′, F(ab′)₂, or a single chain variable fragment (scFv). In some embodiments, the first binding domain is a Fab, Fab′, F(ab′)₂, or a single chain variable fragment (scFv). In some embodiments, the first binding domain comprises the scFv. In some embodiments, the VH of the first binding domain and VL of the first binding domain are connected by an scFv linker sequence. In some embodiments, the scFv linker sequence of the first binding domain comprises an amino acid sequence according to SEQ ID No: 12. In some embodiments, the scFv of the first binding domain comprises an amino acid sequence with at least 80%, 85%, 90%, 95%, 98%, 99%, or 100% sequence identity to SEQ ID NO: 13 or SEQ ID NO: 14. In some embodiments, the first binding domain comprises a constant domain. In some embodiments, the constant domain of the first binding domain comprises a human IgG4 sequence. In some embodiments, the human IgG4 sequence of the first binding domain comprises a S228P point mutation wherein the amino acid numbering is according to the EU index in Kabat et al. In some embodiments, the human IgG4 sequence of the first binding domain does not have a C-terminal lysine residue. In some embodiments, the constant domain of the first binding domain comprises a human IgG1 sequence. In some embodiments, the constant domain of the first binding domain comprises a C_{H}1, C_{H}2, C_{H}3, or a C_{L} amino acid sequence. In some embodiments, the first binding domain comprises a C_{H}1, C_{H}2, C_{H}3, and a C_{L} amino acid sequence. In some embodiments, the first binding domain comprises an amino acid sequence with at least 80%, 85%, 90%, 95%, 98%, 99%, or 100% sequence identity to SEQ ID NO: 15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26. In some embodiments, the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the second binding domain comprises a Fab, Fab′, F(ab′)₂, or a single chain variable fragment (scFv). In some embodiments, the second binding domain is a Fab, Fab′, F(ab′)₂, or a single chain variable fragment (scFv). In some embodiments, the second binding domain comprises the scFv. In some embodiments, the VH of the second binding domain and VL of the second binding domain are connected by an scFv linker sequence. In some embodiments, the scFv linker sequence of the second binding domain comprises an amino acid sequence according to SEQ ID NO: 29. In some embodiments, the scFv of the second binding domain comprises an amino acid sequence with at least 80%, 85%, 90%, 95%, 98%, 99%, or 100% sequence identity to SEQ ID NO: 30 or SEQ ID NO: 31. In some embodiments, the second binding domain comprises a constant domain. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43. In some embodiments, the VL of the third binding domain comprises an amino acid sequence with at least 80%, 85%, 90%, 95%, 98%, 99%, or 100% sequence identity to SEQ ID NO: 44. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44. In some embodiments, the third binding domain comprises a Fab, Fab′, F(ab′)₂, or a single chain variable fragment (scFv). In some embodiments, the third binding domain is a Fab, Fab′, F(ab′)₂, or a single chain variable fragment (scFv). In some embodiments, the third binding domain comprises the scFv. In some embodiments, the VH of the third binding domain and VL of the third binding domain are connected by an scFv linker sequence. In some embodiments, the scFv linker sequence of the third binding domain comprises an amino acid sequence according to SEQ ID NO: 45. In some embodiments, the scFv of the third binding domain comprises an amino acid sequence with at least 80%, 85%, 90%, 95%, 98%, 99%, or 100% sequence identity to SEQ ID NO: 46 or SEQ ID NO: 47. In some embodiments, the third binding domain comprises a constant domain. In some embodiments, the constant domain of the third binding domain comprises a human IgG4 sequence. In some embodiments, the human IgG4 sequence of the third binding domain comprises a S228P point mutation wherein the amino acid numbering is according to the EU index in Kabat et al. In some embodiments, the human IgG4 sequence of the third binding domain does not have a C-terminal lysine residue. In some embodiments, the constant domain of the third binding domain comprises a human IgG1 sequence. In some embodiments, the constant domain of the third binding domain comprises a C_{H}1, C_{H}2, C_{H}3, or a C_{L} amino acid sequence. In some embodiments, the third binding domain comprises a C_{H}1, C_{H}2, C_{H}3, and a C_{L} amino acid sequence. In some embodiments, the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50. In some embodiments, the first binding domain, second binding domain, and the third binding domain are connected within the same molecule. In some embodiments, the first binding domain, second binding domain, and the third binding domain are separate molecules. In some embodiments, the first binding domain and second binding domain are within the same molecule and the third binding domain is a separate molecule from the molecule with the first binding domain and the second binding domain. In some embodiments, the first binding domain and third binding domain are within the same molecule and the second binding domain is a separate molecule from the molecule with the first binding domain and the third binding domain. In some embodiments, the second binding domain and third binding domain are within the same molecule and the first binding domain is a separate molecule from the molecule with the second binding domain and the third binding domain. In some embodiments, the first binding domain comprises a scFv and the second binding domain comprises a C_{H}1, C_{H}2, C_{H}3, and a C_{L} amino acid sequence. In some embodiments, the second binding domain comprises a scFv and the first binding domain comprises a C_{H}1, C_{H}2, C_{H}3, and a C_{L} amino acid sequence. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the composition comprises a pharmaceutically acceptable excipient.

Disclosed herein are isolated recombinant nucleic acid sequences encoding an amino acid sequence of the composition according to any one of the above embodiments.

Disclosed herein are vectors comprising the isolated recombinant nucleic acid sequences according to the above embodiment.

Disclosed herein are kits that comprise at least one of: the composition of any one of the above embodiments; the vector of the above embodiment; or the nucleic acid molecule of the above embodiment.

Disclosed herein are methods of treating a food allergy in a subject in need thereof comprising administering to the subject the composition according to any one of the above embodiments.

Disclosed herein are methods of treating a food allergy in a subject in need thereof comprising administering to the subject the composition according to any one of the above embodiments. In some embodiments, the food allergy comprises a peanut allergy.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the disclosure are utilized, and the accompanying drawings of which:

FIG. 1 illustrates the peanut antigen specificity distribution of mAbs discovered from peanut allergic individuals.

FIG. 2 illustrates epitope binning of Ara h 2-specific mAbs.

FIG. 3A illustrates a simplified ELISA for assessing the ability of IgG mAbs to block the binding of allergic plasma IgE to a tagged allergen.

FIG. 3B illustrates inhibition of recombinant Ara h 2 binding to peanut allergic plasma IgE by one or more Molecule-2 mAbs.

FIG. 3C illustrates binding of Molecule-1 and Molecule-2 mAbs to recombinant Ara h 2.

FIG. 3D illustrates Molecule-1 and Molecule-2 mAbs inhibiting recombinant Ara h 2 binding to peanut allergic plasma IgE.

FIG. 4A illustrates an overview of the mast cell activation test (MAT).

FIG. 4B illustrates percent inhibition of peanut-mediated and Ara h 2-mediated mast cell activation for IgG1 and IgG4 mAbs alone or in combination.

FIG. 5A illustrates Combination-1 comprising two molecules.

FIG. 5B illustrates blocking of recombinant Ara h 2 binding to peanut allergic plasma IgE by Combination-1.

FIG. 5C illustrates inhibition of peanut-mediated mast cell activation by Combination-1 and its constituent components.

FIG. 6A illustrates a protocol for a mouse model of peanut allergy oral sensitization/oral peanut challenge.

FIG. 6B illustrates the dose-dependent effects of Combination-1 and its constituent components on the hypothermic response in peanut allergic animals when challenged with 25 mg of peanut protein delivered via oral gavage.

FIG. 6C illustrates the dose-dependent effects of Combination-1 and its constituent components on serum MCPT-1 in peanut allergic animals when challenged with 25 mg of peanut protein delivered via oral gavage.

### DETAILED DESCRIPTION

Allergies are characterized by a number of conditions caused by hypersensitivity of the immune system to typically harmless substances in the environment. In general, an allergic reaction occurs when aspects of the immune system overreact to the presence of a substance (an allergen) that, absent the allergy, would not cause a reaction. Food, insect bites, and medications are common causes of severe allergic reactions. In addition, there are also many significant non-food allergies, including, but not limited to, pollen (e.g., ragweed, trees, and grasses), animals (e.g., animal dander), molds, metals, and latex.

As generally understood, an allergen is a type of antigen that produces an abnormally vigorous immune response in which the immune system fights off a perceived threat that would otherwise be harmless. In technical terms, an allergen is an antigen that is capable of stimulating a type-I hypersensitivity reaction in atopic individuals through Immunoglobulin E (IgE) responses. Most humans mount significant IgE responses only as a defense against parasitic infections. However, some individuals may respond to many common environmental antigens. This hereditary predisposition is called atopy. In atopic individuals, non-parasitic antigens stimulate inappropriate IgE production, leading to type I hypersensitivity.

Some foods such as peanuts (a legume), nuts, seafood and shellfish are the cause of serious allergies in many people. Officially, the United States Food and Drug Administration does recognize nine foods as being common for allergic reactions in a large segment of the sensitive population. These include peanuts, tree nuts, eggs, milk, shellfish, fish, sesame, wheat and their derivatives, and soy and their derivatives, as well as sulfites (chemical-based, often found in flavors and colors in foods).

An allergic reaction can be caused by any form of direct contact with the allergen-consuming food or drink one is sensitive to (ingestion), breathing in pollen, perfume or pet dander (inhalation), or brushing a body part against an allergy-causing plant (direct contact). An extremely serious form of an allergic reaction is called anaphylaxis.

Immunoglobulin E (IgE) antibodies mediate the allergic response. They bind to specific receptors on inflammatory immune cells, including mast cells in mucosal tissues lining body surfaces and cavities, as well as basophils in the circulation. These cells mediate allergic responses triggered by specific antigens (allergens) that are recognized by IgE through the release of inflammatory molecules, such as histamine. The inflammatory response is responsible for symptoms, such as sneezing, runny or stuffed nose, itchy eyes, breathing difficulties, and, in extreme cases, anaphylactic shock and even death.

Food allergies have increased over time. The most common food allergens include soy products, tree nuts (almond, cashew, walnut, pecan, pistachio, brazil, macadamia, etc.), peanuts, eggs, shellfish, fish, milk, sesame, and wheat. Food allergies have a negative impact on quality of life and further result in a significant economic burden. For example, people suffering from allergies are required to be hypervigilant and may avoid situations, including social interactions, that could result in allergic reactions. Furthermore, for people with multi-food allergies, the risk of severe reactions and anaphylaxis is increased. For many allergies, there is currently no cure and individuals must practice lifelong avoidance. Treatments for allergies include the avoidance of known allergens, as well as the use of medications such as steroids and antihistamines. In severe reactions, injectable adrenaline (epinephrine) is recommended as a rescue treatment. One treatment approach for allergies is immunotherapy. Immunotherapy involves the repeated injection or exposure of allergen extracts to desensitize a patient to the allergen. However, immunotherapy is time consuming, usually involving years of treatment, and often fails to achieve its goal of desensitizing the patient to the allergen. Furthermore, immunotherapy carries the risk of potentially severe adverse events, including anaphylaxis. Accordingly, there is a need for improved therapies for treating allergic response.

Disclosed herein are compositions for preventing, treating and suppressing allergic responses associated with specific allergens. The compositions provide formulations of high affinity, allergen-specific antibodies designed to alleviate and/or prevent an allergic response associated with specific allergens.

### Compositions

Antibodies that Bind to Ara h 2 and Ara h 6

Disclosed herein are isolated antibodies that bind to Ara h 2 and Ara h 6 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8. Disclosed herein are isolated antibodies that bind to Ara h 2 and Ara h 6 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8. In some embodiments, the VH comprises an amino acid sequence with at least 80%, 85%, 90%, 95%, 98%, 99%, or 100% sequence identity to SEQ ID NO: 9 or SEQ ID NO: 10. In some embodiments, the VL comprises an amino acid sequence with at least 80%, 85%, 90%, 95%, 98%, 99%, or 100% sequence identity to SEQ ID NO: 11. In some embodiments, the VH comprises an amino acid sequence with at least 80%, 85%, 90%, 95%, 98%, 99%, or 100% sequence identity to SEQ ID NO: 9 or SEQ ID NO: 10 and wherein the VL comprises an amino acid sequence with at least 80%, 85%, 90%, 95%, 98%, 99%, or 100% sequence identity to SEQ ID NO: 11. In some embodiments, the isolated antibody comprises a Fab, Fab′, F(ab′)₂, or a single chain variable fragment (scFv). In some embodiments, the isolated antibody is a Fab, Fab′, F(ab′)₂, or a single chain variable fragment (scFv). In some embodiments, the isolated antibody comprises the scFv. In some embodiments, the VH and VL are connected by an scFv linker sequence. In some embodiments, the scFv linker sequence comprises an amino acid sequence according to SEQ ID No: 12. In some embodiments, the scFv comprises an amino acid sequence with at least 80%, 85%, 90%, 95%, 98%, 99%, or 100% sequence identity to SEQ ID NO: 13 or SEQ ID NO: 14. In some embodiments, the isolated antibody comprises a constant domain. In some embodiments, the constant domain comprises a human IgG4 sequence. In some embodiments, the human IgG4 sequence comprises a S228P point mutation wherein the amino acid numbering is according to the EU index in Kabat et al. In some embodiments, the human IgG4 sequence does not have a C-terminal lysine residue. In some embodiments, the constant domain comprises a human IgG1 sequence. In some embodiments, the constant domain comprises a C_{H}1, C_{H}2, C_{H}3, or a C_{L} amino acid sequence. In some embodiments, the isolated antibody comprises a C_{H}1, C_{H}2, C_{H}3, and a C_{L} amino acid sequence. In some embodiments, the isolated antibody comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the isolated antibody comprises an amino acid sequence with at least 80%, 85%, 90%, 95%, 98%, 99%, or 100% sequence identity to SEQ ID NO: 17.

TABLE 1
Amino Acid Sequences of Antibodies
that Bind to Ara h 2 and Ara h 6;
CDRs are determined using IMGT domain system.

| | | SEQ |
|---|---|---|
| | | ID |
| Construct Description | Amino Acid Sequence | NO: |
| Binder-1: HC: CDR1 | GFKFSDHW | 1 |
| Binder-1: HC: CDR2 #1 | ISEAGREK | 2 |
| Binder-1: HC: CDR2 #2 | ISEDGREK | 3 |
| Binder-1: HC: CDR3 #1 | ARVSTRVVNAGFDI | 4 |
| Binder-1: HC: CDR3 #2 | ARVSTRVVNDGFDI | 5 |
| Binder-1: LC: CDR1 | NIADKN | 6 |
| Binder-1: LC: CDR2 | DDD | 7 |
| Binder-1: LC: CDR3 | QVWDSRSEYV | 8 |
| Binder-1: heavy chain | EVALVESGGGLVQPGGSLRLSCT | 9 |
| variable domain #1 | ASGFKFSDHWMTWVRQAPGKGLE | |
| | WVASISEAGREKVYVDSVRGRFT | |
| | LSRDNAKKSLSLQMTSLRVDDTA | |
| | KYHCARVSTRVVNAGFDIWGQGA | |
| | MVIVSL | |
| Binder-1: heavy chain | EVALVESGGGLVQPGGSLRLSCT | 10 |
| variable domain #2, | ASGFKFSDHWMTWVRQAPGKGLE | |
| pre-CMC-liability- | WVASISEDGREKVYVDSVRGRFT | |
| removal (D54A/D106A) | LSRDNAKKSLSLQMTSLRVDDTA | |
| | KYHCARVSTRVVNDGFDIWGQGA | |
| | MVIVSL | |
| Binder-1: light chain | SYVLTQPPSVSAAPGETARITCG | 11 |
| variable domain | GKNIADKNVHWYQHRPGQAPVMV | |
| | IYDDDDRPSEIPDRESGSNSGNT | |
| | ATLTISRVEAGDEADYYCQVWDS | |
| | RSEYVFGTGTTVTVL | |
| scFv linker sequence | GGGSGGGGSGGGGSGGGGS | 12 |
| Binder-1: scFv #1 | EVALVESGGGLVQPGGSLRLSCT | 13 |
| (VH then VL) | ASGFKFSDHWMTWVRQAPGKGLE | |
| | WVASISEAGREKVYVDSVRGRFT | |
| | LSRDNAKKSLSLQMTSLRVDDTA | |
| | KYHCARVSTRVVNAGFDIWGQGA | |
| | MVIVSLGGGSGGGGSGGGGSGGG | |
| | GSSYVLTQPPSVSAAPGETARIT | |
| | CGGKNIADKNVHWYQHRPGQAPV | |
| | MVIYDDDDRPSEIPDRFSGSNSG | |
| | NTATLTISRVEAGDEADYYCQVW | |
| | DSRSEYVFGTGTTVTVL | |
| Binder-1: scFv #2 | SYVLTQPPSVSAAPGETARITCG | 14 |
| (VL then VH) | GKNIADKNVHWYQHRPGQAPVMV | |
| | IYDDDDRPSEIPDRFSGSNSGNT | |
| | ATLTISRVEAGDEADYYCQVWDS | |
| | RSEYVFGTGTTVTVLGGGSGGGG | |
| | SGGGGSGGGGSEVALVESGGGLV | |
| | QPGGSLRLSCTASGFKFSDHWMT | |
| | WVRQAPGKGLEWVASISEAGREK | |
| | VYVDSVRGRFTLSRDNAKKSLSL | |
| | QMTSLRVDDTAKYHCARVSTRVV | |
| | NAGFDIWGQGAMVIVSL | |
| Binder-1: full length | EVALVESGGGLVQPGGSLRLSCT | 15 |
| heavy chain #1 | ASGFKFSDHWMTWVRQAPGKGLE | |
| (no c-terminal K) | WVASISEAGREKVYVDSVRGRFT | |
| | LSRDNAKKSLSLQMTSLRVDDTA | |
| | KYHCARVSTRVVNAGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLG | |
| Binder-1: full length | EVALVESGGGLVQPGGSLRLSCT | 16 |
| heavy chain #2 | ASGFKFSDHWMTWVRQAPGKGLE | |
| (with c-terminal K) | WVASISEAGREKVYVDSVRGRFT | |
| | LSRDNAKKSLSLQMTSLRVDDTA | |
| | KYHCARVSTRVVNAGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGK | |
| Molecule-2d: Binder-1: | EVALVESGGGLVQPGGSLRLSCT | 72 |
| full length heavy | ASGFKFSDHWMTWVRQAPGKGLE | |
| chain #3 | WVASISEDGREKVYVDSVRGRFT | |
| | LSRDNAKKSLSLQMTSLRVDDTA | |
| | KYHCARVSTRVVNDGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGK | |
| Molecule-2g: Binder-1: | EVALVESGGGLVQPGGSLRLSCT | 73 |
| full length heavy | ASGFKFSDHWMTWVRQAPGKGLE | |
| chain #3 | WVASISEDGREKVYVDSVRGRFT | |
| | LSRDNAKKSLSLQMTSLRVDDTA | |
| | KYHCARVSTRVVNDGFDIWGQGA | |
| | MVIVSLASTKGPSVFPLAPSSKS | |
| | TSGGTAALGCLVKDYFPEPVTVS | |
| | WNSGALTSGVHTFPAVLQSSGLY | |
| | SLSSVVTVPSSSLGTQTYICNVN | |
| | HKPSNTKVDKKVEPKSCDKTHTC | |
| | PPCPAPELLGGPSVFLFPPKPKD | |
| | TLMISRTPEVTCVVVDVSHEDPE | |
| | VKFNWYVDGVEVHNAKTKPREEQ | |
| | YNSTYRVVSVLTVLHQDWLNGKE | |
| | YKCKVSNKALPAPIEKTISKAKG | |
| | QPREPQVYTLPPSRDELTKNQVS | |
| | LTCLVKGFYPSDIAVEWESNGQP | |
| | ENNYKTTPPVLDSDGSFFLYSKL | |
| | TVDKSRWQQGNVFSCSVMHEALH | |
| | NHYTQKSLSLSPGK | |
| Binder-1: full length | SYVLTQPPSVSAAPGETARITCG | 17 |
| light chain | GKNIADKNVHWYQHRPGQAPVMV | |
| | IYDDDDRPSEIPDRESGSNSGNT | |
| | ATLTISRVEAGDEADYYCQVWDS | |
| | RSEYVFGTGTTVTVLGQPKAAPS | |
| | VTLFPPSSEELQANKATLVCLIS | |
| | DFYPGAVTVAWKADSSPVKAGVE | |
| | TTTPSKQSNNKYAASSYLSLTPE | |
| | QWKSHRSYSCQVTHEGSTVEKTV | |
| | APTECS | |

Disclosed herein are antibodies that bind to Ara h 2 and Ara h 6 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24. Disclosed herein are antibodies that bind to Ara h 2 and Ara h 6 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24. In some embodiments, the VH comprises an amino acid sequence with at least 80%, 85%, 90%, 95%, 98%, 99%, or 100% sequence identity to SEQ ID NO: 25 or SEQ ID NO: 26. In some embodiments, the VL comprises an amino acid sequence with at least 80%, 85%, 90%, 95%, 98%, 99%, or 100% sequence identity to SEQ ID NO: 27 or SEQ ID NO: 28. In some embodiments, the VH comprises an amino acid sequence with at least 80%, 85%, 90%, 95%, 98%, 99%, or 100% sequence identity to SEQ ID NO: 25 or SEQ ID NO: 26 and wherein the VL comprises an amino acid sequence with at least 80%, 85%, 90%, 95%, 98%, 99%, or 100% sequence identity to SEQ ID NO: 27 or SEQ ID NO: 28. In some embodiments, the isolated antibody comprises a Fab, Fab′, F(ab′)₂, or a single chain variable fragment (scFv). In some embodiments, the isolated antibody is a Fab, Fab′, F(ab′)₂, or a single chain variable fragment (scFv). In some embodiments, the isolated antibody comprises the scFv. In some embodiments, the VH and VL are connected by an scFv linker sequence. In some embodiments, the scFv linker sequence comprises an amino acid sequence according to SEQ ID NO: 29. In some embodiments, the scFv comprises an amino acid sequence with at least 80%, 85%, 90%, 95%, 98%, 99%, or 100% sequence identity to SEQ ID NO: 30 or SEQ ID NO: 31. In some embodiments, the isolated antibody comprises a constant domain. In some embodiments, the constant domain comprises a human IgG4 sequence. In some embodiments, the human IgG4 sequence comprises a S228P point mutation wherein the amino acid numbering is according to the EU index in Kabat et al. In some embodiments, the human IgG4 sequence does not have a C-terminal lysine residue. In some embodiments, the constant domain comprises a human IgG1 sequence. In some embodiments, the constant domain comprises a C_{H}1, C_{H}2, C_{H}3, or a C_{L} amino acid sequence. In some embodiments, the isolated antibody comprises a C_{H}1, C_{H}2, C_{H}3, and a C_{L} amino acid sequence. In some embodiments, the isolated antibody comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

TABLE 2
Amino Acid Sequences of Antibodies that Bind to
Ara h 2 and Ara h 6; CDRs are determined using
IMGT domain system.

| | | SEQ |
|---|---|---|
| | | ID |
| Construct Description | Amino Acid Sequence | NO: |
| Binder-2: HC: CDR1 | GFNEDDYT | 18 |
| Binder-2: HC: CDR2 | IRYNSVRI | 19 |
| Binder-2: HC: CDR3 #1 | VKDEGERSFEI | 20 |
| Binder-2: HC: CDR3 #2 | VKDNGFRSFEI | 21 |
| Binder-2: LC: CDR1 | QSLEHSNGYHY | 22 |
| Binder-2: LC: CDR2 | LGS | 23 |
| Binder-2: LC: CDR3 | MQALQTWT | 24 |
| Binder-2: heavy chain | EVQLVESGGGVVQPGRSLTLTCV | 25 |
| variable domain #1 | GSGFNEDDYTMHWVRQRPGKGLE | |
| | WVSSIRYNSVRIEYVESVKGRFT | |
| | TSRDNAKNSLYMEMKSLRPEDTA | |
| | FYYCVKDEGFRSFEIWGQGTMVI | |
| | VSS | |
| Binder-2: heavy chain | EVQLVESGGGVVQPGRSLTLTCV | 26 |
| variable domain #2, | GSGFNFDDYTMHWVRQRPGKGLE | |
| pre-CMC-liability- | WVSSIRYNSVRIEYVESVKGRFT | |
| removal (N100E) | TSRDNAKNSLYMEMKSLRPEDTA | |
| | FYYCVKDNGFRSFEIWGQGTMVI | |
| | VSS | |
| Binder-2: light chain | DIVMTQSPLSLPVTPGEPASISC | 27 |
| variable domain #1 | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIK | |
| Binder-2: light chain | EIVMTQSPLSLPVTPGEPASISC | 28 |
| variable domain #2, | RSTQSLEHSNGYHYLDWYVQKAG | |
| pre-CMC-liability- | QSPQLLISLGSKRASGVPDRFSG | |
| removal (E1D) | SVSGTDFTLKISRVEAEDVGVYY | |
| | CMQALQTWTFGQGTKVEIK | |
| scFv linker sequence | GGGSGGGGSGGGGSGGGGS | 29 |
| Binder-2: scFv #1 | EVQLVESGGGVVQPGRSLTLTCV | 30 |
| (VH then VL) | GSGFNEDDYTMHWVRQRPGKGLE | |
| | WVSSIRYNSVRIEYVESVKGRFT | |
| | TSRDNAKNSLYMEMKSLRPEDTA | |
| | FYYCVKDEGFRSFEIWGQGTMVI | |
| | VSSGGGSGGGGSGGGGSGGGGSD | |
| | IVMTQSPLSLPVTPGEPASISCR | |
| | STQSLEHSNGYHYLDWYVQKAGQ | |
| | SPQLLISLGSKRASGVPDRFSGS | |
| | VSGTDFTLKISRVEAEDVGVYYC | |
| | MQALQTWTFGQGTKVEIK | |
| Binder-2: scFv #1 | DIVMTQSPLSLPVTPGEPASISC | 31 |
| (VL then VH) | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKGGGS | |
| | GGGGSGGGGSGGGGSEVQLVESG | |
| | GGVVQPGRSLTLTCVGSGENFDD | |
| | YTMHWVRQRPGKGLEWVSSIRYN | |
| | SVRIEYVESVKGRFTTSRDNAKN | |
| | SLYMEMKSLRPEDTAFYYCVKDE | |
| | GFRSFEIWGQGTMVIVSS | |
| Binder-2: full length | EVQLVESGGGVVQPGRSLTLTCV | 32 |
| heavy chain #1 | GSGFNFDDYTMHWVRQRPGKGLE | |
| (no c-terminal K) | WVSSIRYNSVRIEYVESVKGRFT | |
| | TSRDNAKNSLYMEMKSLRPEDTA | |
| | FYYCVKDEGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLG | |
| Binder-2: full length | EVQLVESGGGVVQPGRSLTLTCV | 33 |
| heavy chain #2 | GSGFNFDDYTMHWVRQRPGKGLE | |
| (with c-terminal K) | WVSSIRYNSVRIEYVESVKGRFT | |
| | TSRDNAKNSLYMEMKSLRPEDTA | |
| | FYYCVKDEGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQENWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGK | |
| Molecule-2e: Binder-2: | EVQLVESGGGVVQPGRSLTLTCV | 74 |
| full length heavy | GSGFNFDDYTMHWVRQRPGKGLE | |
| chain #3 | WVSSIRYNSVRIEYVESVKGRFT | |
| | TSRDNAKNSLYMEMKSLRPEDTA | |
| | FYYCVKDNGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGK | |
| Molecule-2h: Binder-2: | EVQLVESGGGVVQPGRSLTLTCV | 75 |
| full length heavy | GSGFNFDDYTMHWVRQRPGKGLE | |
| chain #4 | WVSSIRYNSVRIEYVESVKGRFT | |
| | TSRDNAKNSLYMEMKSLRPEDTA | |
| | FYYCVKDNGFRSFEIWGQGTMVI | |
| | VSSASTKGPSVFPLAPSSKSTSG | |
| | GTAALGCLVKDYFPEPVTVSWNS | |
| | GALTSGVHTFPAVLQSSGLYSLS | |
| | SVVTVPSSSLGTQTYICNVNHKP | |
| | SNTKVDKKVEPKSCDKTHTCPPC | |
| | PAPELLGGPSVFLFPPKPKDTLM | |
| | ISRTPEVTCVVVDVSHEDPEVKF | |
| | NWYVDGVEVHNAKTKPREEQYNS | |
| | TYRVVSVLTVLHQDWLNGKEYKC | |
| | KVSNKALPAPIEKTISKAKGQPR | |
| | EPQVYTLPPSRDELTKNQVSLTC | |
| | LVKGFYPSDIAVEWESNGQPENN | |
| | YKTTPPVLDSDGSFFLYSKLTVD | |
| | KSRWQQGNVFSCSVMHEALHNHY | |
| | TQKSLSLSPGK | |
| Binder-2: full length | DIVMTQSPLSLPVTPGEPASISC | 34 |
| light chain | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSFNRGEC | |
| Molecule-2e & Molecule- | EIVMTQSPLSLPVTPGEPASISC | 76 |
| 2h: Binder-2: full | RSTQSLEHSNGYHYLDWYVQKAG | |
| length light chain #2 | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSFNRGEC | |

Antibodies that Bind to Ara h 2

Disclosed herein are isolated antibodies that bind to Ara h 2 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. Disclosed herein are isolated antibodies that bind to Ara h 2 that comprise a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH comprises an amino acid sequence with at least 80%, 85%, 90%, 95%, 98%, 99%, or 100% sequence identity to SEQ ID NO: 42 or SEQ ID NO: 43. In some embodiments, the VL comprises an amino acid sequence with at least 80%, 85%, 90%, 95%, 98%, 99%, or 100% sequence identity to SEQ ID NO: 44. In some embodiments, the VH comprises an amino acid sequence with at least 80%, 85%, 90%, 95%, 98%, 99%, or 100% sequence identity to SEQ ID NO: 42 or SEQ ID NO: 43 and wherein the VL comprises an amino acid sequence with at least 80%, 85%, 90%, 95%, 98%, 99%, or 100% sequence identity to SEQ ID NO: 44. In some embodiments, the isolated antibody comprises a Fab, Fab′, F(ab′)₂, or a single chain variable fragment (scFv). In some embodiments, the isolated antibody is a Fab, Fab′, F(ab′)₂, or a single chain variable fragment (scFv). In some embodiments, the isolated antibody comprises the scFv. In some embodiments, the VH and VL are connected by an scFv linker sequence. In some embodiments, the scFv linker sequence comprises an amino acid sequence according to SEQ ID NO: 45. In some embodiments, the scFv comprises an amino acid sequence with at least 80%, 85%, 90%, 95%, 98%, 99%, or 100% sequence identity to SEQ ID NO: 46 or SEQ ID NO: 47. In some embodiments, the isolated antibody comprises a constant domain. In some embodiments, the constant domain comprises a human IgG4 sequence. In some embodiments, the human IgG4 sequence comprises a S228P point mutation wherein the amino acid numbering is according to the EU index in Kabat et al. In some embodiments, the human IgG4 sequence does not have a C-terminal lysine residue. In some embodiments, the constant domain comprises a human IgG1 sequence. In some embodiments, the constant domain comprises a C_{H}1, C_{H}2, C_{H}3, or a C_{L} amino acid sequence. In some embodiments, the isolated antibody comprises a C_{H}1, C_{H}2, C_{H}3, and a C_{L} amino acid sequence. In some embodiments, the isolated antibody comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

TABLE 3
Amino Acid Sequences of Antibodies that
Bind to Ara h 2; CDRs are determined
using IMGT domain system.

| | | SEQ |
|---|---|---|
| | | ID |
| Construct Description | Amino Acid Sequence | NO: |
| Binder-3: HC: CDR1 | GFTFSYHA | 35 |
| Binder-3: HC: CDR2 #1 | ISDAGDRR | 36 |
| Binder-3: HC: CDR2 #2 | ISDNGNRR | 37 |
| Binder-3: HC: CDR3 | ARTMDFDSGSHYYGLDV | 38 |
| Binder-3: LC: CDR1 | QTINSNY | 39 |
| Binder-3: LC: CDR2 | LTS | 40 |
| Binder-3: LC: CDR3 | QHYSRSPPYS | 41 |
| Binder-3: heavy chain | EVQLVQSGGGVVQPGRSLTVSCA | 42 |
| variable domain #1 | GSGFTFSYHALHWVRQAPGKGLE | |
| | WLAVISDAGDRRDYVDSVRGRFS | |
| | VSRDNSRNTVFLQMDSLRADDSA | |
| | VYFCARTMDFDSGSHYYGLDVWG | |
| | QGTTVIVSS | |
| Binder-3: heavy chain | QEQLVQSGGGVVQPGRSLTVSCA | 43 |
| variable domain #2, | GSGFTFSYHALHWVRQAPGKGLE | |
| pre-CMC-liability- | WLAVISDNGNRRDYVDSVRGRES | |
| removal (Q1E/E2V/N54A/ | VSRDNSRNTVFLQMDSLRADDSA | |
| N56D) | VYFCARTMDFDSGSHYYGLDVWG | |
| | QGTTVIVSS | |
| Binder-3: light chain | EVVLTQSPALLSLSPGERASLSC | 44 |
| variable domain | RASQTINSNYLAWYQLKPGQAPR | |
| | LVIYLTSTRAPGIPARFSGSGSG | |
| | TDFTLTIDRLEPEDFALYYCQHY | |
| | SRSPPYSFGQGTRLDVK | |
| scFv linker sequence | GGGSGGGGSGGGGSGGGGS | 45 |
| Binder-3: scFv #1 | EVQLVQSGGGVVQPGRSLTVSCA | 46 |
| (VH then VL) | GSGFTFSYHALHWVRQAPGKGLE | |
| | WLAVISDAGDRRDYVDSVRGRFS | |
| | VSRDNSRNTVFLQMDSLRADDSA | |
| | VYFCARTMDFDSGSHYYGLDVWG | |
| | QGTTVIVSSGGGSGGGGSGGGGS | |
| | GGGGSEVVLTQSPALLSLSPGER | |
| | ASLSCRASQTINSNYLAWYQLKP | |
| | GQAPRLVIYLTSTRAPGIPARFS | |
| | GSGSGTDFTLTIDRLEPEDFALY | |
| | YCQHYSRSPPYSFGQGTRLDVK | |
| Binder-3: scFv #2 | EVVLTQSPALLSLSPGERASLSC | 47 |
| (VL then VH) | RASQTINSNYLAWYQLKPGQAPR | |
| | LVIYLTSTRAPGIPARFSGSGSG | |
| | TDFTLTIDRLEPEDFALYYCQHY | |
| | SRSPPYSFGQGTRLDVKGGGSGG | |
| | GGSGGGGSGGGGSEVQLVQSGGG | |
| | VVQPGRSLTVSCAGSGFTFSYHA | |
| | LHWVRQAPGKGLEWLAVISDAGD | |
| | RRDYVDSVRGRFSVSRDNSRNTV | |
| | FLQMDSLRADDSAVYFCARTMDF | |
| | DSGSHYYGLDVWGQGTTVIVSS | |
| Molecule-2c: Binder-3: | EVQLVQSGGGVVQPGRSLTVSCA | 48 |
| full length heavy | GSGFTFSYHALHWVRQAPGKGLE | |
| chain #1 (no | WLAVISDAGDRRDYVDSVRGRFS | |
| c-terminal K) | VSRDNSRNTVFLQMDSLRADDSA | |
| | VYFCARTMDFDSGSHYYGLDVWG | |
| | QGTTVIVSSASTKGPSVFPLAPC | |
| | SRSTSESTAALGCLVKDYFPEPV | |
| | TVSWNSGALTSGVHTFPAVLQSS | |
| | GLYSLSSVVTVPSSSLGTKTYTC | |
| | NVDHKPSNTKVDKRVESKYGPPC | |
| | PPCPAPEFLGGPSVFLFPPKPKD | |
| | TLMISRTPEVTCVVVDVSQEDPE | |
| | VQFNWYVDGVEVHNAKTKPREEQ | |
| | FNSTYRVVSVLTVLHQDWLNGKE | |
| | YKCKVSNKGLPSSIEKTISKAKG | |
| | QPREPQVYTLPPSQEEMTKNQVS | |
| | LTCLVKGFYPSDIAVEWESNGQP | |
| | ENNYKTTPPVLDSDGSFFLYSRL | |
| | TVDKSRWQEGNVFSCSVMHEALH | |
| | NHYTQKSLSLSLG | |
| Binder-3: full length | EVQLVQSGGGVVQPGRSLTVSCA | 49 |
| heavy chain #2 | GSGFTFSYHALHWVRQAPGKGLE | |
| (with c-terminal K) | WLAVISDAGDRRDYVDSVRGRFS | |
| | VSRDNSRNTVFLQMDSLRADDSA | |
| | VYFCARTMDFDSGSHYYGLDVWG | |
| | QGTTVIVSSASTKGPSVFPLAPC | |
| | SRSTSESTAALGCLVKDYFPEPV | |
| | TVSWNSGALTSGVHTFPAVLQSS | |
| | GLYSLSSVVTVPSSSLGTKTYTC | |
| | NVDHKPSNTKVDKRVESKYGPPC | |
| | PPCPAPEFLGGPSVFLFPPKPKD | |
| | TLMISRTPEVTCVVVDVSQEDPE | |
| | VQFNWYVDGVEVHNAKTKPREEQ | |
| | FNSTYRVVSVLTVLHQDWLNGKE | |
| | YKCKVSNKGLPSSIEKTISKAKG | |
| | QPREPQVYTLPPSQEEMTKNQVS | |
| | LTCLVKGFYPSDIAVEWESNGQP | |
| | ENNYKTTPPVLDSDGSFFLYSRL | |
| | TVDKSRWQEGNVFSCSVMHEALH | |
| | NHYTQKSLSLSLGK | |
| Molecule-2f: Binder-3: | QEQLVQSGGGVVQPGRSLTVSCA | 77 |
| full length heavy | GSGFTFSYHALHWVRQAPGKGLE | |
| chain #3 | WLAVISDNGNRRDYVDSVRGRFS | |
| | VSRDNSRNTVFLQMDSLRADDSA | |
| | VYFCARTMDFDSGSHYYGLDVWG | |
| | QGTTVIVSSASTKGPSVFPLAPC | |
| | SRSTSESTAALGCLVKDYFPEPV | |
| | TVSWNSGALTSGVHTFPAVLQSS | |
| | GLYSLSSVVTVPSSSLGTKTYTC | |
| | NVDHKPSNTKVDKRVESKYGPPC | |
| | PPCPAPEFLGGPSVFLFPPKPKD | |
| | TLMISRTPEVTCVVVDVSQEDPE | |
| | VQFNWYVDGVEVHNAKTKPREEQ | |
| | FNSTYRVVSVLTVLHQDWLNGKE | |
| | YKCKVSNKGLPSSIEKTISKAKG | |
| | QPREPQVYTLPPSQEEMTKNQVS | |
| | LTCLVKGFYPSDIAVEWESNGQP | |
| | ENNYKTTPPVLDSDGSFFLYSRL | |
| | TVDKSRWQEGNVFSCSVMHEALH | |
| | NHYTQKSLSLSLGK | |
| Molecule-2i: Binder-3: | QEQLVQSGGGVVQPGRSLTVSCA | 78 |
| full length heavy | GSGFTFSYHALHWVRQAPGKGLE | |
| chain #4 | WLAVISDNGNRRDYVDSVRGRFS | |
| | VSRDNSRNTVFLQMDSLRADDSA | |
| | VYFCARTMDFDSGSHYYGLDVWG | |
| | QGTTVIVSSASTKGPSVFPLAPS | |
| | SKSTSGGTAALGCLVKDYFPEPV | |
| | TVSWNSGALTSGVHTFPAVLQSS | |
| | GLYSLSSVVTVPSSSLGTQTYIC | |
| | NVNHKPSNTKVDKKVEPKSCDKT | |
| | HTCPPCPAPELLGGPSVFLFPPK | |
| | PKDTLMISRTPEVTCVVVDVSHE | |
| | DPEVKFNWYVDGVEVHNAKTKPR | |
| | EEQYNSTYRVVSVLTVLHQDWLN | |
| | GKEYKCKVSNKALPAPIEKTISK | |
| | AKGQPREPQVYTLPPSRDELTKN | |
| | QVSLTCLVKGFYPSDIAVEWESN | |
| | GQPENNYKTTPPVLDSDGSFFLY | |
| | SKLTVDKSRWQQGNVFSCSVMHE | |
| | ALHNHYTQKSLSLSPGK | |
| Molecule-2c: Binder-3: | EVVLTQSPALLSLSPGERASLSC | 50 |
| full length light | RASQTINSNYLAWYQLKPGQAPR | |
| chain | LVIYLTSTRAPGIPARFSGSGSG | |
| | TDFTLTIDRLEPEDFALYYCQHY | |
| | SRSPPYSFGQGTRLDVKRTVAAP | |
| | SVFIFPPSDEQLKSGTASVVCLL | |
| | NNFYPREAKVQWKVDNALQSGNS | |
| | QESVTEQDSKDSTYSLSSTLTLS | |
| | KADYEKHKVYACEVTHQGLSSPV | |
| | TKSFNRGEC | |

Antibodies with a First Binding Domain and a Second Binding Domain

Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 and Ara h 6 wherein the second binding domain comprises a VH that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24. Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 and Ara h 6 wherein the second binding domain comprises a VH that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10. In some embodiments, the VL of the first binding domain comprises an amino acid sequence with at least 80%, 85%, 90%, 95%, 98%, 99%, or 100% sequence identity to SEQ ID NO: 11. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11. In some embodiments, the first binding domain comprises a Fab, Fab′, F(ab′)₂, or a single chain variable fragment (scFv). In some embodiments, the first binding domain is a Fab, Fab′, F(ab′)₂, or a single chain variable fragment (scFv). In some embodiments, the first binding domain comprises the scFv. In some embodiments, the VH of the first binding domain and VL of the first binding domain are connected by an scFv linker sequence. In some embodiments, the scFv linker sequence of the first binding domain comprises an amino acid sequence according to SEQ ID No: 12. In some embodiments, the scFv of the first binding domain comprises an amino acid sequence with at least 80%, 85%, 90%, 95%, 98%, 99%, or 100% sequence identity to SEQ ID NO: 13 or SEQ ID NO: 14. In some embodiments, the first binding domain comprises a constant domain. In some embodiments, the constant domain of the first binding domain comprises a human IgG4 sequence. In some embodiments, the human IgG4 sequence of the first binding domain comprises a S228P point mutation wherein the amino acid numbering is according to the EU index in Kabat et al. In some embodiments, the human IgG4 sequence of the first binding domain does not have a C-terminal lysine residue. In some embodiments, the constant domain of the first binding domain comprises a human IgG1 sequence. In some embodiments, the constant domain of the first binding domain comprises a C_{H}1, C_{H}2, C_{H}3, or a C_{L} amino acid sequence. In some embodiments, the first binding domain comprises a C_{H}1, C_{H}2, C_{H}3, and a C_{L} amino acid sequence. In some embodiments, the first binding domain comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26. In some embodiments, the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the second binding domain comprises a Fab, Fab′, F(ab′)₂, or a single chain variable fragment (scFv). In some embodiments, the second binding domain is a Fab, Fab′, F(ab′)₂, or a single chain variable fragment (scFv). In some embodiments, the second binding domain comprises the scFv. In some embodiments, the VH of the second binding domain and VL of the second binding domain are connected by an scFv linker sequence. In some embodiments, the scFv linker sequence of the second binding domain comprises an amino acid sequence according to SEQ ID NO: 29. In some embodiments, the scFv of the second binding domain comprises an amino acid sequence with at least 80%, 85%, 90%, 95%, 98%, 99%, or 100% sequence identity to SEQ ID NO: 30 or SEQ ID NO: 31. In some embodiments, the second binding domain comprises a constant domain. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76. In some embodiments, the first binding domain comprises the scFv and the second binding domain comprises a C_{H}1, C_{H}2, C_{H}3, and a C_{L} amino acid sequence. In some embodiments, the second binding domain comprises the scFv and the first binding domain comprises a C_{H}1, C_{H}2, C_{H}3, and a C_{L} amino acid sequence. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the isolated antibody comprises an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52. In some embodiments, the isolated antibody comprises an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54. In some embodiments, the isolated antibody comprises an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64. In some embodiments, the isolated antibody comprises an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66. In some embodiments, the isolated antibody comprises an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68. In some embodiments, the isolated antibody comprises an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70.

Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10. In some embodiments, the VL of the first binding domain comprises an amino acid sequence with at least 80%, 85%, 90%, 95%, 98%, 99%, or 100% sequence identity to SEQ ID NO: 11. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11. In some embodiments, the first binding domain comprises a Fab, Fab′, F(ab′)₂, or a single chain variable fragment (scFv). In some embodiments, the first binding domain is a Fab, Fab′, F(ab′)₂, or a single chain variable fragment (scFv). In some embodiments, the first binding domain comprises the scFv. In some embodiments, the VH of the first binding domain and VL of the first binding domain are connected by an scFv linker sequence. In some embodiments, the scFv linker sequence of the first binding domain comprises an amino acid sequence according to SEQ ID No: 12. In some embodiments, the scFv of the first binding domain comprises an amino acid sequence with at least 80%, 85%, 90%, 95%, 98%, 99%, or 100% sequence identity to SEQ ID NO: 13 or SEQ ID NO: 14. In some embodiments, the first binding domain comprises a constant domain. In some embodiments, the constant domain of the first binding domain comprises a human IgG4 sequence. In some embodiments, the human IgG4 sequence of the first binding domain comprises a S228P point mutation wherein the amino acid numbering is according to the EU index in Kabat et al. In some embodiments, the human IgG4 sequence of the first binding domain does not have a C-terminal lysine residue. In some embodiments, the constant domain of the first binding domain comprises a human IgG1 sequence. In some embodiments, the constant domain of the first binding domain comprises a C_{H}1, C_{H}2, C_{H}3, or a C_{L} amino acid sequence. In some embodiments, the first binding domain comprises a C_{H}1, C_{H}2, C_{H}3, and a C_{L} amino acid sequence. In some embodiments, the first binding domain comprises an amino acid sequence with at least 80%, 85%, 90%, 95%, 98%, 99%, or 100% sequence identity to SEQ ID NO: 15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17. In some embodiments, the VH of the second binding domain comprises an amino acid sequence with at least 80%, 85%, 90%, 95%, 98%, 99%, or 100% sequence identity to SEQ ID NO: 42 or SEQ ID NO: 43. In some embodiments, the VL of the second binding domain comprises an amino acid sequence with at least 80%, 85%, 90%, 95%, 98%, 99%, or 100% sequence identity to SEQ ID NO: 44. In some embodiments, the VH of the second binding domain comprises an amino acid sequence with at least 80%, 85%, 90%, 95%, 98%, 99%, or 100% sequence identity to SEQ ID NO: 42 or SEQ ID NO: 43 and wherein the VL of the second binding domain comprises an amino acid sequence with at least 80%, 85%, 90%, 95%, 98%, 99%, or 100% sequence identity to SEQ ID NO: 44. In some embodiments, the second binding domain comprises a Fab, Fab′, F(ab′)₂, or a single chain variable fragment (scFv). In some embodiments, the second binding domain is a Fab, Fab′, F(ab′)₂, or a single chain variable fragment (scFv). In some embodiments, the second binding domain comprises the scFv. In some embodiments, the VH of the second binding domain and VL of the second binding domain are connected by an scFv linker sequence. In some embodiments, the scFv linker sequence of the second binding domain comprises an amino acid sequence according to SEQ ID NO: 45. In some embodiments, the scFv of the second binding domain comprises an amino acid sequence with at least 80%, 85%, 90%, 95%, 98%, 99%, or 100% sequence identity to SEQ ID NO: 46 or SEQ ID NO: 47. In some embodiments, the second binding domain comprises a constant domain. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50. In some embodiments, the first binding domain comprises the scFv and the second binding domain comprises a C_{H}1, C_{H}2, C_{H}3, and a C_{L} amino acid sequence. In some embodiments, the second binding domain comprises the scFv and the first binding domain comprises a C_{H}1, C_{H}2, C_{H}3, and a C_{L} amino acid sequence. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the isolated antibody comprises an amino acid sequence with at least 80%, 85%, 90%, 95%, 98%, 99%, or 100% sequence identity to SEQ ID NO: 57 and an amino acid sequence with at least 80%, 85%, 90%, 95%, 98%, 99%, or 100% sequence identity to SEQ ID NO: 56. In some embodiments, the isolated antibody comprises an amino acid sequence with at least 80%, 85%, 90%, 95%, 98%, 99%, or 100% sequence identity to SEQ ID NO: 59 and an amino acid sequence with at least 80%, 85%, 90%, 95%, 98%, 99%, or 100% sequence identity to SEQ ID NO: 58.

Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. Disclosed herein are isolated antibodies that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH of the first binding domain comprises an amino acid sequence with at least 80%, 85%, 90%, 95%, 98%, 99%, or 100% sequence identity to SEQ ID NO: 25 or SEQ ID NO: 26. In some embodiments, the VL of the first binding domain comprises an amino acid sequence with at least 80%, 85%, 90%, 95%, 98%, 99%, or 100% sequence identity to SEQ ID NO: 27 or SEQ ID NO: 28. In some embodiments, the VH of the first binding domain comprises an amino acid sequence with at least 80%, 85%, 90%, 95%, 98%, 99%, or 100% sequence identity to SEQ ID NO: 25 or SEQ ID NO: 26 and wherein the VL of the first binding domain comprises an amino acid sequence with at least 80%, 85%, 90%, 95%, 98%, 99%, or 100% sequence identity to SEQ ID NO: 27 or SEQ ID NO: 28. In some embodiments, the first binding domain comprises a Fab, Fab′, F(ab′)₂, or a single chain variable fragment (scFv). In some embodiments, the first binding domain is a Fab, Fab′, F(ab′)₂, or a single chain variable fragment (scFv). In some embodiments, the first binding domain comprises the scFv. In some embodiments, the VH of the first binding domain and VL of the first binding domain are connected by an scFv linker sequence. In some embodiments, the scFv linker sequence of the first binding domain comprises an amino acid sequence according to SEQ ID NO: 29. In some embodiments, the scFv of the first binding domain comprises an amino acid sequence with at least 80%, 85%, 90%, 95%, 98%, 99%, or 100% sequence identity to SEQ ID NO: 30 or SEQ ID NO: 31. In some embodiments, the first binding domain comprises a constant domain. In some embodiments, the constant domain of the first binding domain comprises a human IgG4 sequence. In some embodiments, the human IgG4 sequence of the first binding domain comprises a S228P point mutation wherein the amino acid numbering is according to the EU index in Kabat et al. In some embodiments, the human IgG4 sequence of the first binding domain does not have a C-terminal lysine residue. In some embodiments, the constant domain of the first binding domain comprises a human IgG1 sequence. In some embodiments, the constant domain of the first binding domain comprises a C_{H}1, C_{H}2, C_{H}3, or a C_{L} amino acid sequence. In some embodiments, the first binding domain comprises a C_{H}1, C_{H}2, C_{H}3, and a C_{L} amino acid sequence. In some embodiments, the first binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76. In some embodiments, the VH of the second binding domain comprises an amino acid sequence with at least 80%, 85%, 90%, 95%, 98%, 99%, or 100% sequence identity to SEQ ID NO: 42 or SEQ ID NO: 43. In some embodiments, the VL of the second binding domain comprises an amino acid sequence with at least 80%, 85%, 90%, 95%, 98%, 99%, or 100% sequence identity to SEQ ID NO: 44. In some embodiments, the VH of the second binding domain comprises an amino acid sequence with at least 80%, 85%, 90%, 95%, 98%, 99%, or 100% sequence identity to SEQ ID NO: 42 or SEQ ID NO: 43 and wherein the VL of the second binding domain comprises an amino acid sequence with at least 80%, 85%, 90%, 95%, 98%, 99%, or 100% sequence identity to SEQ ID NO: 44. In some embodiments, the second binding domain comprises a Fab, Fab′, F(ab′)₂, or a single chain variable fragment (scFv). In some embodiments, the second binding domain is a Fab, Fab′, F(ab′)₂, or a single chain variable fragment (scFv). In some embodiments, the second binding domain comprises the scFv. In some embodiments, the VH of the second binding domain and VL of the second binding domain are connected by an scFv linker sequence. In some embodiments, the scFv linker sequence of the second binding domain comprises an amino acid sequence according to SEQ ID NO: 45. In some embodiments, the scFv of the second binding domain comprises an amino acid sequence with at least 80%, 85%, 90%, 95%, 98%, 99%, or 100% sequence identity to SEQ ID NO: 46 or SEQ ID NO: 47. In some embodiments, the second binding domain comprises a constant domain. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50. In some embodiments, the first binding domain comprises the scFv and the second binding domain comprises a C_{H}1, C_{H}2, C_{H}3, and a C_{L} amino acid sequence. In some embodiments, the second binding domain comprises the scFv and the first binding domain comprises a C_{H}1, C_{H}2, C_{H}3, and a C_{L} amino acid sequence. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the isolated antibody comprises an amino acid sequence with at least 80%, 85%, 90%, 95%, 98%, 99%, or 100% sequence identity to SEQ ID NO: 61 and an amino acid sequence with at least 80%, 85%, 90%, 95%, 98%, 99%, or 100% sequence identity to SEQ ID NO: 60. In some embodiments, the isolated antibody comprises an amino acid sequence with at least 80%, 85%, 90%, 95%, 98%, 99%, or 100% sequence identity to SEQ ID NO: 63 and an amino acid sequence with at least 80%, 85%, 90%, 95%, 98%, 99%, or 100% sequence identity to SEQ ID NO: 62.

TABLE 4
Amino Acid Sequences of Antibodies with
a First Binding Domain and a Second
Binding Domain

| Construct | | SEQ |
|---|---|---|
| | | ID |
| Description | Amino Acid Sequence | NO: |
| Linking | GGGGSGGGGS | 51 |
| moiety-1 | | |
| Molecule-1a | DIVMTQSPLSLPVTPGEPASISC | 52 |
| Light Chain | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSFNRGEC | |
| Molecule-1a | EVQLVESGGGVVQPGRSLTLTCV | 53 |
| Heavy Chain + | GSGFNFDDYTMHWVRQRPGKGLE | |
| scFv | WVSSIRYNSVRIEYVESVKGRFT | |
| | TSRDNAKNSLYMEMKSLRPEDTA | |
| | FYYCVKDEGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGKGGGGGSGGGSEVALV | |
| | ESGGGLVQPGGSLRLSCTASGFK | |
| | FSDHWMTWVRQAPGKGLEWVASI | |
| | SEAGREKVYVDSVRGRFTLSRDN | |
| | AKKSLSLQMTSLRVDDTAKYHCA | |
| | RVSTRVVNAGFDIWGQGAMVIVS | |
| | LGGGSGGGGSGGGGSGGGGSSYV | |
| | LTQPPSVSAAPGETARITCGGKN | |
| | IADKNVHWYQHRPGQAPVMVIYD | |
| | DDDRPSEIPDRFSGSNSGNTATL | |
| | TISRVEAGDEADYYCQVWDSRSE | |
| | YVFGTGTTVTVL | |
| Molecule-1b | SYVLTQPPSVSAAPGETARITCG | 54 |
| Light Chain | GKNIADKNVHWYQHRPGQAPVMV | |
| | IYDDDDRPSEIPDRFSGSNSGNT | |
| | ATLTISRVEAGDEADYYCQVWDS | |
| | RSEYVFGTGTTVTVLGQPKAAPS | |
| | VTLFPPSSEELQANKATLVCLIS | |
| | DFYPGAVTVAWKADSSPVKAGVE | |
| | TTTPSKQSNNKYAASSYLSLTPE | |
| | QWKSHRSYSCQVTHEGSTVEKTV | |
| | APTECS | |
| Molecule-1b | EVALVESGGGLVQPGGSLRLSCT | 55 |
| Heavy Chain + | ASGFKFSDHWMTWVRQAPGKGLE | |
| scFv | WVASISEAGREKVYVDSVRGRET | |
| | LSRDNAKKSLSLQMTSLRVDDTA | |
| | KYHCARVSTRVVNAGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQENS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGKGGGGSGGGGSEV | |
| | QLVESGGGVVQPGRSLTLTCVGS | |
| | GFNFDDYTMHWVRQRPGKGLEWV | |
| | SSIRYNSVRIEYVESVKGRFTTS | |
| | RDNAKNSLYMEMKSLRPEDTAFY | |
| | YCVKDEGFRSFEIWGQGTMVIVS | |
| | SGGGSGGGGSGGGGSGGGGSDIV | |
| | MTQSPLSLPVTPGEPASISCRST | |
| | QSLEHSNGYHYLDWYVQKAGQSP | |
| | QLLISLGSKRASGVPDRFSGSVS | |
| | GTDFTLKISRVEAEDVGVYYCMQ | |
| | ALQTWTFGQGTKVEIK | |
| Molecule-1c | SYVLTQPPSVSAAPGETARITCG | 56 |
| Light Chain | GKNIADKNVHWYQHRPGQAPVMV | |
| | IYDDDDRPSEIPDRESGSNSGNT | |
| | ATLTISRVEAGDEADYYCQVWDS | |
| | RSEYVFGTGTTVTVLGQPKAAPS | |
| | VTLFPPSSEELQANKATLVCLIS | |
| | DFYPGAVTVAWKADSSPVKAGVE | |
| | TTTPSKQSNNKYAASSYLSLTPE | |
| | QWKSHRSYSCQVTHEGSTVEKTV | |
| | APTECS | |
| Molecule-1c | EVALVESGGGLVQPGGSLRLSCT | 57 |
| Heavy Chain + | ASGFKFSDHWMTWVRQAPGKGLE | |
| scFv | WVASISEAGREKVYVDSVRGRFT | |
| | LSRDNAKKSLSLQMTSLRVDDTA | |
| | KYHCARVSTRVVNAGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGKGGGGSGGGGSEV | |
| | QLVQSGGGVVQPGRSLTVSCAGS | |
| | GFTFSYHALHWVRQAPGKGLEWL | |
| | AVISDAGDRRDYVDSVRGRESVS | |
| | RDNSRNTVFLQMDSLRADDSAVY | |
| | FCARTMDFDSGSHYYGLDVWGQG | |
| | TTVIVSSGGGSGGGGSGGGGSGG | |
| | GGSEVVLTQSPALLSLSPGERAS | |
| | LSCRASQTINSNYLAWYQLKPGQ | |
| | APRLVIYLTSTRAPGIPARFSGS | |
| | GSGTDFTLTIDRLEPEDFALYYC | |
| | QHYSRSPPYSFGQGTRLDVK | |
| Molecule-1d | EVVLTQSPALLSLSPGERASLSC | 58 |
| Light Chain | RASQTINSNYLAWYQLKPGQAPR | |
| | LVIYLTSTRAPGIPARFSGSGSG | |
| | TDFTLTIDRLEPEDFALYYCQHY | |
| | SRSPPYSFGQGTRLDVKRTVAAP | |
| | SVFIFPPSDEQLKSGTASVVCLL | |
| | NNFYPREAKVQWKVDNALQSGNS | |
| | QESVTEQDSKDSTYSLSSTLTLS | |
| | KADYEKHKVYACEVTHQGLSSPV | |
| | TKSFNRGEC | |
| Molecule-1d | EVQLVQSGGGVVQPGRSLTVSCA | 59 |
| Heavy Chain + | GSGFTFSYHALHWVRQAPGKGLE | |
| scFv | WLAVISDAGDRRDYVDSVRGRES | |
| | VSRDNSRNTVFLQMDSLRADDSA | |
| | VYFCARTMDFDSGSHYYGLDVWG | |
| | QGTTVIVSSASTKGPSVFPLAPC | |
| | SRSTSESTAALGCLVKDYFPEPV | |
| | TVSWNSGALTSGVHTFPAVLQSS | |
| | GLYSLSSVVTVPSSSLGTKTYTC | |
| | NVDHKPSNTKVDKRVESKYGPPC | |
| | PPCPAPEFLGGPSVFLFPPKPKD | |
| | TLMISRTPEVTCVVVDVSQEDPE | |
| | VQFNWYVDGVEVHNAKTKPREEQ | |
| | FNSTYRVVSVLTVLHQDWLNGKE | |
| | YKCKVSNKGLPSSIEKTISKAKG | |
| | QPREPQVYTLPPSQEEMTKNQVS | |
| | LTCLVKGFYPSDIAVEWESNGQP | |
| | ENNYKTTPPVLDSDGSFFLYSRL | |
| | TVDKSRWQEGNVFSCSVMHEALH | |
| | NHYTQKSLSLSLGKGGGGSGGGG | |
| | SEVALVESGGGLVQPGGSLRLSC | |
| | TASGFKFSDHWMTWVRQAPGKGL | |
| | EWVASISEAGREKVYVDSVRGRF | |
| | TLSRDNAKKSLSLQMTSLRVDDT | |
| | AKYHCARVSTRVVNAGFDIWGQG | |
| | AMVIVSLGGGSGGGGSGGGGSGG | |
| | GGSSYVLTQPPSVSAAPGETARI | |
| | TCGGKNIADKNVHWYQHRPGQAP | |
| | VMVIYDDDDRPSEIPDRFSGSNS | |
| | GNTATLTISRVEAGDEADYYCQV | |
| | WDSRSEYVFGTGTTVTVL | |
| Molecule-1e | DIVMTQSPLSLPVTPGEPASISC | 60 |
| Light Chain | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSENRGEC | |
| Molecule-1e | EVQLVESGGGVVQPGRSLTLTCV | 61 |
| Heavy Chain + | GSGFNFDDYTMHWVRQRPGKGLE | |
| scFv | WVSSIRYNSVRIEYVESVKGRFT | |
| | TSRDNAKNSLYMEMKSLRPEDTA | |
| | FYYCVKDEGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGKGGGGSGGGGSEVQLV | |
| | QSGGGVVQPGRSLTVSCAGSGFT | |
| | FSYHALHWVRQAPGKGLEWLAVI | |
| | SDAGDRRDYVDSVRGRFSVSRDN | |
| | SRNTVFLQMDSLRADDSAVYFCA | |
| | RTMDFDSGSHYYGLDVWGQGTTV | |
| | IVSSGGGSGGGGSGGGGSGGGGS | |
| | EVVLTQSPALLSLSPGERASLSC | |
| | RASQTINSNYLAWYQLKPGQAPR | |
| | LVIYLTSTRAPGIPARFSGSGSG | |
| | TDFTLTIDRLEPEDFALYYCQHY | |
| | SRSPPYSFGQGTRLDVK | |
| Molecule-1f | EVVLTQSPALLSLSPGERASLSC | 62 |
| Light Chain | RASQTINSNYLAWYQLKPGQAPR | |
| | LVIYLTSTRAPGIPARFSGSGSG | |
| | TDFTLTIDRLEPEDFALYYCQHY | |
| | SRSPPYSFGQGTRLDVKRTVAAP | |
| | SVFIFPPSDEQLKSGTASVVCLL | |
| | NNFYPREAKVQWKVDNALQSGNS | |
| | QESVTEQDSKDSTYSLSSTLTLS | |
| | KADYEKHKVYACEVTHQGLSSPV | |
| | TKSFNRGEC | |
| Molecule-1f | EVQLVQSGGGVVQPGRSLTVSCA | 63 |
| Heavy Chain + | GSGFTFSYHALHWVRQAPGKGLE | |
| scFv | WLAVISDAGDRRDYVDSVRGRES | |
| | VSRDNSRNTVFLQMDSLRADDSA | |
| | VYFCARTMDFDSGSHYYGLDVWG | |
| | QGTTVIVSSASTKGPSVFPLAPC | |
| | SRSTSESTAALGCLVKDYFPEPV | |
| | TVSWNSGALTSGVHTFPAVLQSS | |
| | GLYSLSSVVTVPSSSLGTKTYTC | |
| | NVDHKPSNTKVDKRVESKYGPPC | |
| | PPCPAPEFLGGPSVFLFPPKPKD | |
| | TLMISRTPEVTCVVVDVSQEDPE | |
| | VQFNWYVDGVEVHNAKTKPREEQ | |
| | FNSTYRVVSVLTVLHQDWLNGKE | |
| | YKCKVSNKGLPSSIEKTISKAKG | |
| | QPREPQVYTLPPSQEEMTKNQVS | |
| | LTCLVKGFYPSDIAVEWESNGQP | |
| | ENNYKTTPPVLDSDGSFFLYSRL | |
| | TVDKSRWQEGNVFSCSVMHEALH | |
| | NHYTQKSLSLSLGKGGGGSGGGG | |
| | SEVQLVESGGGVVQPGRSLTLTC | |
| | VGSGFNEDDYTMHWVRQRPGKGL | |
| | EWVSSIRYNSVRIEYVESVKGRF | |
| | TTSRDNAKNSLYMEMKSLRPEDT | |
| | AFYYCVKDEGFRSFEIWGQGTMV | |
| | IVSSGGGSGGGGSGGGGSGGGGS | |
| | DIVMTQSPLSLPVTPGEPASISC | |
| | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIK | |
| Molecule-1g | EIVMTQSPLSLPVTPGEPASISC | 64 |
| Light Chain | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSFNRGEC | |
| Molecule-1g | EVQLVESGGGVVQPGRSLTLTCV | 65 |
| Heavy Chain + | GSGFNEDDYTMHWVRQRPGKGLE | |
| scFv | WVSSIRYNSVRIEYVESVKGRFT | |
| | TSRDNAKNSLYMEMKSLRPEDTA | |
| | FYYCVKDNGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGKGGGGSGGGGSEVALV | |
| | ESGGGLVQPGGSLRLSCTASGFK | |
| | FSDHWMTWVRQAPGKGLEWVASI | |
| | SEDGREKVYVDSVRGRFTLSRDN | |
| | AKKSLSLQMTSLRVDDTAKYHCA | |
| | RVSTRVVNDGFDIWGQGAMVIVS | |
| | LGGGSGGGGSGGGGSGGGGSSYV | |
| | LTQPPSVSAAPGETARITCGGKN | |
| | IADKNVHWYQHRPGQAPVMVIYD | |
| | DDDRPSEIPDRFSGSNSGNTATL | |
| | TISRVEAGDEADYYCQVWDSRSE | |
| | YVFGTGTTVTVL | |
| Molecule-1h | EIVMTQSPLSLPVTPGEPASISC | 66 |
| Light Chain | RSTQSLEHSNGYHYLDWYVQKAG | |
| | QSPQLLISLGSKRASGVPDRFSG | |
| | SVSGTDFTLKISRVEAEDVGVYY | |
| | CMQALQTWTFGQGTKVEIKRTVA | |
| | APSVFIFPPSDEQLKSGTASVVC | |
| | LLNNFYPREAKVQWKVDNALQSG | |
| | NSQESVTEQDSKDSTYSLSSTLT | |
| | LSKADYEKHKVYACEVTHQGLSS | |
| | PVTKSFNRGEC | |
| Molecule-1h | EVQLVESGGGVVQPGRSLTLTCV | 67 |
| Heavy Chain + | GSGFNFDDYTMHWVRQRPGKGLE | |
| scFv | WVSSIRYNSVRIEYVESVKGRFT | |
| | TSRDNAKNSLYMEMKSLRPEDTA | |
| | FYYCVKDNGFRSFEIWGQGTMVI | |
| | VSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRVESKYGPPCPPCPAP | |
| | EFLGGPSVFLFPPKPKDTLMISR | |
| | TPEVTCVVVDVSQEDPEVQFNWY | |
| | VDGVEVHNAKTKPREEQFNSTYR | |
| | VVSVLTVLHQDWLNGKEYKCKVS | |
| | NKGLPSSIEKTISKAKGQPREPQ | |
| | VYTLPPSQEEMTKNQVSLTCLVK | |
| | GFYPSDIAVEWESNGQPENNYKT | |
| | TPPVLDSDGSFFLYSRLTVDKSR | |
| | WQEGNVFSCSVMHEALHNHYTQK | |
| | SLSLSLGKGGGGSGGGGSSYVLT | |
| | QPPSVSAAPGETARITCGGKNIA | |
| | DKNVHWYQHRPGQAPVMVIYDDD | |
| | DRPSEIPDRFSGSNSGNTATLTI | |
| | SRVEAGDEADYYCQVWDSRSEYV | |
| | FGTGTTVTVLGGGSGGGGSGGGG | |
| | SGGGGSEVALVESGGGLVQPGGS | |
| | LRLSCTASGFKFSDHWMTWVRQA | |
| | PGKGLEWVASISEDGREKVYVDS | |
| | VRGRFTLSRDNAKKSLSLQMTSL | |
| | RVDDTAKYHCARVSTRVVNDGED | |
| | IWGQGAMVIVSL | |
| Molecule-1i | SYVLTQPPSVSAAPGETARITCG | 68 |
| Light Chain | GKNIADKNVHWYQHRPGQAPVMV | |
| | IYDDDDRPSEIPDRFSGSNSGNT | |
| | ATLTISRVEAGDEADYYCQVWDS | |
| | RSEYVFGTGTTVTVLGQPKAAPS | |
| | VTLFPPSSEELQANKATLVCLIS | |
| | DFYPGAVTVAWKADSSPVKAGVE | |
| | TTTPSKQSNNKYAASSYLSLTPE | |
| | QWKSHRSYSCQVTHEGSTVEKTV | |
| | APTECS | |
| Molecule-1i | EVALVESGGGLVQPGGSLRLSCT | 69 |
| Heavy Chain + | ASGFKFSDHWMTWVRQAPGKGLE | |
| scFv | WVASISEDGREKVYVDSVRGRFT | |
| | LSRDNAKKSLSLQMTSLRVDDTA | |
| | KYHCARVSTRVVNDGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGKGGGGGGGGSEV | |
| | QLVESGGGVVQPGRSLTLTCVGS | |
| | GFNEDDYTMHWVRQRPGKGLEWV | |
| | SSIRYNSVRIEYVESVKGRETTS | |
| | RDNAKNSLYMEMKSLRPEDTAFY | |
| | YCVKDNGFRSFEIWGQGTMVIVS | |
| | SGGGSGGGGSGGGGSGGGGSEIV | |
| | MTQSPLSLPVTPGEPASISCRST | |
| | QSLEHSNGYHYLDWYVQKAGQSP | |
| | QLLISLGSKRASGVPDRFSGSVS | |
| | GTDFTLKISRVEAEDVGVYYCMQ | |
| | ALQTWTFGQGTKVEIK | |
| Molecule-1j | SYVLTQPPSVSAAPGETARITCG | 70 |
| Light Chain | GKNIADKNVHWYQHRPGQAPVMV | |
| | IYDDDDRPSEIPDRFSGSNSGNT | |
| | ATLTISRVEAGDEADYYCQVWDS | |
| | RSEYVFGTGTTVTVLGQPKAAPS | |
| | VTLFPPSSEELQANKATLVCLIS | |
| | DFYPGAVTVAWKADSSPVKAGVE | |
| | TTTPSKQSNNKYAASSYLSLTPE | |
| | QWKSHRSYSCQVTHEGSTVEKTV | |
| | APTECS | |
| Molecule-1j | EVALVESGGGLVQPGGSLRLSCT | 71 |
| Heavy Chain + | ASGFKFSDHWMTWVRQAPGKGLE | |
| scFv | WVASISEDGREKVYVDSVRGRFT | |
| | LSRDNAKKSLSLQMTSLRVDDTA | |
| | KYHCARVSTRVVNDGFDIWGQGA | |
| | MVIVSLASTKGPSVFPLAPCSRS | |
| | TSESTAALGCLVKDYFPEPVTVS | |
| | WNSGALTSGVHTFPAVLQSSGLY | |
| | SLSSVVTVPSSSLGTKTYTCNVD | |
| | HKPSNTKVDKRVESKYGPPCPPC | |
| | PAPEFLGGPSVFLFPPKPKDTLM | |
| | ISRTPEVTCVVVDVSQEDPEVQF | |
| | NWYVDGVEVHNAKTKPREEQFNS | |
| | TYRVVSVLTVLHQDWLNGKEYKC | |
| | KVSNKGLPSSIEKTISKAKGQPR | |
| | EPQVYTLPPSQEEMTKNQVSLTC | |
| | LVKGFYPSDIAVEWESNGQPENN | |
| | YKTTPPVLDSDGSFFLYSRLTVD | |
| | KSRWQEGNVFSCSVMHEALHNHY | |
| | TQKSLSLSLGKGGGGSGGGGSEI | |
| | VMTQSPLSLPVTPGEPASISCRS | |
| | TQSLEHSNGYHYLDWYVQKAGQS | |
| | PQLLISLGSKRASGVPDRFSGSV | |
| | SGTDFTLKISRVEAEDVGVYYCM | |
| | QALQTWTFGQGTKVEIKGGGSGG | |
| | GGSGGGGSGGGGSEVQLVESGGG | |
| | VVQPGRSLTLTCVGSGFNFDDYT | |
| | MHWVRQRPGKGLEWVSSIRYNSV | |
| | RIEYVESVKGRFTTSRDNAKNSL | |
| | YMEMKSLRPEDTAFYYCVKDNGF | |
| | RSFEIWGQGTMVIVSS | |

Compositions with a First Binding Domain, a Second Binding Domain, and a Third Binding Domain

Disclosed herein are compositions that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. Disclosed herein are compositions that comprise a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10. In some embodiments, the VL of the first binding domain comprises an amino acid sequence with at least 80%, 85%, 90%, 95%, 98%, 99%, or 100% sequence identity to SEQ ID NO: 11. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11. In some embodiments, the first binding domain comprises a Fab, Fab′, F(ab′)₂, or a single chain variable fragment (scFv). In some embodiments, the first binding domain is a Fab, Fab′, F(ab′)₂, or a single chain variable fragment (scFv). In some embodiments, the first binding domain comprises the scFv. In some embodiments, the VH of the first binding domain and VL of the first binding domain are connected by an scFv linker sequence. In some embodiments, the scFv linker sequence of the first binding domain comprises an amino acid sequence according to SEQ ID No: 12. In some embodiments, the scFv of the first binding domain comprises an amino acid sequence with at least 80%, 85%, 90%, 95%, 98%, 99%, or 100% sequence identity to SEQ ID NO: 13 or SEQ ID NO: 14. In some embodiments, the first binding domain comprises a constant domain. In some embodiments, the constant domain of the first binding domain comprises a human IgG4 sequence. In some embodiments, the human IgG4 sequence of the first binding domain comprises a S228P point mutation wherein the amino acid numbering is according to the EU index in Kabat et al. In some embodiments, the human IgG4 sequence of the first binding domain does not have a C-terminal lysine residue. In some embodiments, the constant domain of the first binding domain comprises a human IgG1 sequence. In some embodiments, the constant domain of the first binding domain comprises a C_{H}1, C_{H}2, C_{H}3, or a C_{L} amino acid sequence. In some embodiments, the first binding domain comprises a C_{H}1, C_{H}2, C_{H}3, and a C_{L} amino acid sequence. In some embodiments, the first binding domain comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26. In some embodiments, the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the second binding domain comprises a Fab, Fab′, F(ab′)₂, or a single chain variable fragment (scFv). In some embodiments, the second binding domain is a Fab, Fab′, F(ab′)₂, or a single chain variable fragment (scFv). In some embodiments, the second binding domain comprises the scFv. In some embodiments, the VH of the second binding domain and VL of the second binding domain are connected by an scFv linker sequence. In some embodiments, the scFv linker sequence of the second binding domain comprises an amino acid sequence according to SEQ ID NO: 29. In some embodiments, the scFv of the second binding domain comprises an amino acid sequence with at least 80%, 85%, 90%, 95%, 98%, 99%, or 100% sequence identity to SEQ ID NO: 30 or SEQ ID NO: 31. In some embodiments, the second binding domain comprises a constant domain. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43. In some embodiments, the VL of the third binding domain comprises an amino acid sequence with at least 80%, 85%, 90%, 95%, 98%, 99%, or 100% sequence identity to SEQ ID NO: 44. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44. In some embodiments, the third binding domain comprises a Fab, Fab′, F(ab′)₂, or a single chain variable fragment (scFv). In some embodiments, the third binding domain is a Fab, Fab′, F(ab′)₂, or a single chain variable fragment (scFv). In some embodiments, the third binding domain comprises the scFv. In some embodiments, the VH of the third binding domain and VL of the third binding domain are connected by an scFv linker sequence. In some embodiments, the scFv linker sequence of the third binding domain comprises an amino acid sequence according to SEQ ID NO: 45. In some embodiments, the scFv of the third binding domain comprises an amino acid sequence with at least 80%, 85%, 90%, 95%, 98%, 99%, or 100% sequence identity to SEQ ID NO: 46 or SEQ ID NO: 47. In some embodiments, the third binding domain comprises a constant domain. In some embodiments, the constant domain of the third binding domain comprises a human IgG4 sequence. In some embodiments, the human IgG4 sequence of the third binding domain comprises a S228P point mutation wherein the amino acid numbering is according to the EU index in Kabat et al. In some embodiments, the human IgG4 sequence of the third binding domain does not have a C-terminal lysine residue. In some embodiments, the constant domain of the third binding domain comprises a human IgG1 sequence. In some embodiments, the constant domain of the third binding domain comprises a C_{H}1, C_{H}2, C_{H}3, or a C_{L} amino acid sequence. In some embodiments, the third binding domain comprises a C_{H}1, C_{H}2, C_{H}3, and a C_{L} amino acid sequence. In some embodiments, the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50. In some embodiments, the first binding domain, second binding domain, and the third binding domain are connected within the same molecule. In some embodiments, the first binding domain, second binding domain, and the third binding domain are separate molecules. In some embodiments, the first binding domain and second binding domain are within the same molecule and the third binding domain is a separate molecule from the molecule with the first binding domain and the second binding domain. In some embodiments, the first binding domain and third binding domain are within the same molecule and the second binding domain is a separate molecule from the molecule with the first binding domain and the third binding domain. In some embodiments, the second binding domain and third binding domain are within the same molecule and the first binding domain is a separate molecule from the molecule with the second binding domain and the third binding domain. In some embodiments, the first binding domain comprises the scFv and the second binding domain comprises a C_{H}1, C_{H}2, C_{H}3, and a C_{L} amino acid sequence. In some embodiments, the second binding domain comprises the scFv and the first binding domain comprises a C_{H}1, C_{H}2, C_{H}3, and a C_{L} amino acid sequence. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Disclosed herein are compositions that comprise a first molecule that comprises a first binding domain that binds to Ara h 2 and Ara h 6 and a second binding domain that binds to Ara h 2 and Ara h 6, wherein the first binding domain comprises a scFv and the second binding domain comprises a heavy chain variable domain, a light chain variable domain, and a constant domain, and the scFv of the first binding domain is connected to the constant domain of the second binding domain by a linking moiety, and the composition comprises a second molecule that comprises a third binding domain that binds to Ara h 2. In some embodiments, the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10. In some embodiments, the VL of the first binding domain comprises an amino acid sequence with at least 80%, 85%, 90%, 95%, 98%, 99%, or 100% sequence identity to SEQ ID NO: 11. In some embodiments, the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11. In some embodiments, the VH of the first binding domain and VL of the first binding domain are connected by an scFv linker sequence. In some embodiments, the scFv linker sequence of the first binding domain comprises an amino acid sequence according to SEQ ID No: 12. In some embodiments, the scFv of the first binding domain comprises an amino acid sequence with at least 80%, 85%, 90%, 95%, 98%, 99%, or 100% sequence identity to SEQ ID NO: 13 or SEQ ID NO: 14. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26. In some embodiments, the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28. In some embodiments, the second binding domain comprises a Fab, Fab′, or a F(ab′)₂. In some embodiments, the second binding domain is a Fab, Fab′, or a F(ab′)₂. In some embodiments, the constant domain of the second binding domain comprises a human IgG4 sequence. In some embodiments, the human IgG4 sequence of the second binding domain comprises a S228P point mutation wherein the amino acid numbering is according to the EU index in Kabat et al. In some embodiments, the human IgG4 sequence of the second binding domain does not have a C-terminal lysine residue. In some embodiments, the constant domain of the second binding domain comprises a human IgG1 sequence. In some embodiments, the constant domain of the second binding domain comprises a C_{H}1, C_{H}2, C_{H}3, or a C_{L} amino acid sequence. In some embodiments, the second binding domain comprises a C_{H}1, C_{H}2, C_{H}3, and a C_{L} amino acid sequence. In some embodiments, the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43. In some embodiments, the VL of the third binding domain comprises an amino acid sequence with at least 80%, 85%, 90%, 95%, 98%, 99%, or 100% sequence identity to SEQ ID NO: 44. In some embodiments, the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44. In some embodiments, the third binding domain comprises a Fab, Fab′, F(ab′)₂, or a single chain variable fragment (scFv). In some embodiments, the third binding domain is a Fab, Fab′, F(ab′)₂, or a single chain variable fragment (scFv). In some embodiments, the third binding domain comprises the scFv. In some embodiments, the VH of the third binding domain and VL of the third binding domain are connected by an scFv linker sequence. In some embodiments, the scFv linker sequence of the third binding domain comprises an amino acid sequence according to SEQ ID NO: 45. In some embodiments, the scFv of the third binding domain comprises an amino acid sequence with at least 80%, 85%, 90%, 95%, 98%, 99%, or 100% sequence identity to SEQ ID NO: 46 or SEQ ID NO: 47. In some embodiments, the third binding domain comprises a constant domain. In some embodiments, the constant domain of the third binding domain comprises a human IgG4 sequence. In some embodiments, the human IgG4 sequence of the third binding domain comprises a S228P point mutation wherein the amino acid numbering is according to the EU index in Kabat et al. In some embodiments, the human IgG4 sequence of the third binding domain does not have a C-terminal lysine residue. In some embodiments, the constant domain of the third binding domain comprises a human IgG1 sequence. In some embodiments, the constant domain of the third binding domain comprises a C_{H}1, C_{H}2, C_{H}3, or a C_{L} amino acid sequence. In some embodiments, the third binding domain comprises a C_{H}1, C_{H}2, C_{H}3, and a C_{L} amino acid sequence. In some embodiments, the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50. In some embodiments, the first binding domain and the second binding domain are connected by a linking moiety. In some embodiments, the linking moiety is an amino acid sequence. In some embodiments, the linking moiety is at least 4 amino acids long. In some embodiments, the linking moiety is between 4-20 amino acids in length. In some embodiments, the linking moiety connects the C_{H}3 amino acid sequence of the second binding domain to the scFv of the first binding domain. In some embodiments, the linking moiety comprises the amino acid sequence of SEQ ID NO: 51. In some embodiments, the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50. In some embodiments, the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

### Pharmaceutical Compositions

Disclosed herein, in some embodiments, are pharmaceutical compositions comprising: (a) the antibodies and compositions as disclosed herein; and (b) a pharmaceutically acceptable excipient.

In some embodiments, the antibody further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the antibody as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disorder to be treated, the age and condition of the individual to be treated, etc. and a physician will ultimately determine appropriate dosages to be used.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or a cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term and high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in the culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins. Expression Vectors

In some embodiments, vectors include any suitable vectors derived from eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary Algae Vectors Include pChlamy-4 Vector or MCS Vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, pcDNA3.4, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes, or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes, or Synergistetes. Other bacteria can be Acidobacteria, Chloroflexi, Chrysiogenetes, Cyanobacteria, Deferribacteres, Dictyoglomi, Thermodesulfobacteria, or Thermotogae. A bacterial cell can be Escherichia coli, Clostridium botulinum, or coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, an animal cell includes a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid, or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293H cells, CHO DG44 cells, CHO-S cells, CHO-K1 cells, FUT8 KO CHO-K1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line or a cell line that has incorporated a genetic material of interest into its own genome and has the capability to express the product of the genetic material after many generations of cell division. In some cases, a mammalian host cell is a transient cell line” or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary algal cell lines include, but are not limited to, strains from Chlamydomonas reinhardtii 137c, or Synechococcus elongatus PPC 7942.

### Methods of Treatment

Disclosed herein are methods of treating a food allergy in a subject in need thereof comprising administering to the subject the isolated antibodies and/or compositions disclosed herein. In some embodiments, the food allergy comprises a peanut allergy. In some embodiments, the subject displays a symptom of peanut allergy. In some embodiments, the symptom of peanut allergy comprises urticaria, vomiting, abdominal pain, coughing, wheezing, or anaphylaxis. In some embodiments, the isolated antibody or composition that is administered to the subject is administered to the subject intravenously (IV), intramuscularly (IM), or subcutaneously.

In some embodiments, the composition comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2, HC-CDR-3: SEQ ID NO: 4, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41. In some embodiments, the first binding domain and second binding domain are within the same molecule and the third binding domain is a separate molecule from the molecule with the first binding domain and the second binding domain. In some embodiments, the first binding domain comprises an scFv and the second binding domain comprises a C_{H}1, C_{H}2, C_{H}3, and a C_{L} amino acid sequence and the first binding domain and second binding domain are connected by a linking moiety to an Fc region of the second binding domain. In some embodiments, the third binding domain that is a separate molecule from the first binding domain and the second binding domain is a monoclonal antibody. In some embodiments, the molecule containing the first binding domain and the second binding domain is administered to the subject before the molecule with the third binding domain. In some embodiments, the molecule containing the first binding domain and the second binding domain is administered to the subject at the same time as the molecule with the third binding domain. In some embodiments, the molecule containing the first binding domain and the second binding domain is administered to the subject after the molecule with the third binding domain.

### Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an antibody that specifically binds to Ara h 2 and/or Ara h 6.

The label or package insert indicates that the composition is used for treating the condition of choice. Moreover, the article of manufacture may comprise (a) a first container with a composition contained therein, wherein the composition comprises the bispecific antibody of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind an antigen (e.g., Fab, F(ab′)₂, Fv, single chain antibodies, single chain variable fragments (scFv), diabodies, antibody chimeras, hybrid antibodies, bispecific antibodies, and the like), and recombinant peptides comprising the forgoing. “Antibody” can mean a monospecific antibody, a bispecific antibody, or a multispecific antibody.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular cytotoxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)₂ fragment's heavy chain disulfide bridge. Other chemical couplings of antibody fragments are also known.

The “light chains” of antibodies (immunoglobulins) from any vertebrate species can be assigned to one of two clearly distinct types, called kappa (x) and lambda (Q), based on the amino acid sequences of their constant domains.

“Single chain variable fragment” or “scFv” refers to a fusion protein of the variable region of a light chain and the variable region of a heavy chain. The light chain variable region can be N-terminal to the heavy chain variable region or the heavy chain variable region can be N-terminal to the light chain variable region.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody are determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of allergic response, alleviation of symptoms, diminishment of any direct or indirect pathological consequences of the allergic response, preventing metastasis, decreasing the rate of allergic response, amelioration or palliation of the allergic response, and remission or improved prognosis. In some embodiments, the molecules of the disclosure are used to delay development of a disease or to slow allergic response.

### EMBODIMENTS

Embodiment A1. An isolated antibody that binds to Ara h 2 and Ara h 6 that comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8.

Embodiment A2. The isolated antibody of Embodiment A1, wherein the VH comprises an amino acid sequence with at least 80%, 85%, 90%, 95%, 98%, 99%, or 100% sequence identity to SEQ ID NO: 9 or SEQ ID NO: 10.

Embodiment A3. The isolated antibody of Embodiments A1 or A2, wherein the VL comprises an amino acid sequence with at least 80%, 85%, 90%, 95%, 98%, 99%, or 100% sequence identity to SEQ ID NO: 11.

Embodiment A4. The isolated antibody of any one of Embodiments A1-A3, wherein the VH comprises an amino acid sequence with at least 80%, 85%, 90%, 95%, 98%, 99%, or 100% sequence identity to SEQ ID NO: 9 or SEQ ID NO: 10 and wherein the VL comprises an amino acid sequence with at least 80%, 85%, 90%, 95%, 98%, 99%, or 100% sequence identity to SEQ ID NO: 11.

Embodiment A5. The isolated antibody of any one of Embodiments A1-A4, wherein the isolated antibody comprises a Fab, Fab′, F(ab′)₂, or a single chain variable fragment (scFv).

Embodiment A6. The isolated antibody of any one of Embodiments A1-A5, wherein the isolated antibody is a Fab, Fab′, F(ab′)₂, or a single chain variable fragment (scFv).

Embodiment A7. The isolated antibody of Embodiments A5 or A6, wherein the isolated antibody comprises the scFv.

Embodiment A8. The isolated antibody of any one of Embodiments A1-A7, wherein the VH and VL are connected by an scFv linker sequence.

Embodiment A9. The isolated antibody of Embodiment A8, wherein the scFv linker sequence comprises an amino acid sequence according to SEQ ID No: 12.

Embodiment A10. The isolated antibody of any one of Embodiments A5-A9, wherein the scFv comprises an amino acid sequence with at least 80%, 85%, 90%, 95%, 98%, 99%, or 100% sequence identity to SEQ ID NO: 13 or SEQ ID NO: 14.

Embodiment A11. The isolated antibody of any one of Embodiments A1-A6, wherein the isolated antibody comprises a constant domain.

Embodiment A12. The isolated antibody of Embodiment A11, wherein the constant domain comprises a human IgG4 sequence.

Embodiment A13. The isolated antibody of Embodiment A12, wherein the human IgG4 sequence comprises a S228P point mutation wherein the amino acid numbering is according to the EU index in Kabat et al.

Embodiment A14. The isolated antibody of Embodiments A12 or A13, wherein the human IgG4 sequence does not have a C-terminal lysine residue.

Embodiment A15. The isolated antibody of any one of Embodiments A11-A14, wherein the constant domain comprises a human IgG1 sequence.

Embodiment A16. The isolated antibody of any one of Embodiments A11-A15, wherein the constant domain comprises a C_{H}1, C_{H}2, C_{H}3, or a C_{L} amino acid sequence.

Embodiment A17. The isolated antibody of any one of Embodiments A1-A15, wherein the isolated antibody comprises a C_{H}1, C_{H}2, C_{H}3, and a C_{L} amino acid sequence.

Embodiment A18. The isolated antibody of any one of Embodiments A1-A17, wherein the isolated antibody comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the isolated antibody comprises an amino acid sequence with at least 80%, 85%, 90%, 95%, 98%, 99%, or 100% sequence identity to SEQ ID NO: 17.

Embodiment A19. An isolated recombinant nucleic acid sequence encoding an amino acid sequence of the isolated antibody according to any one of Embodiments A1-A18.

Embodiment A20. A vector comprising the isolated recombinant nucleic acid sequence according to Embodiment A19.

Embodiment A21. A pharmaceutical composition comprising: (a) the isolated antibody according to any one of Embodiments A1-A18; and (b) a pharmaceutically acceptable excipient.

Embodiment A22. A method of treating a food allergy in a subject in need thereof comprising administering to the subject the isolated antibody according to any one of Embodiments A1-A18.

Embodiment A23. The method of Embodiment A22, wherein the food allergy comprises a peanut allergy.

Embodiment B1. An isolated antibody that binds to Ara h 2 and Ara h 6 that comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24.

Embodiment B2. The isolated antibody of Embodiment B1, wherein the VH comprises an amino acid sequence with at least 80%, 85%, 90%, 95%, 98%, 99%, or 100% sequence identity to SEQ ID NO: 25 or SEQ ID NO: 26.

Embodiment B3. The isolated antibody of Embodiments B1 or B2, wherein the VL comprises an amino acid sequence with at least 80%, 85%, 90%, 95%, 98%, 99%, or 100% sequence identity to SEQ ID NO: 27 or SEQ ID NO: 28.

Embodiment B4. The isolated antibody of any one of Embodiments B1-B3, wherein the VH comprises an amino acid sequence with at least 80%, 85%, 90%, 95%, 98%, 99%, or 100% sequence identity to SEQ ID NO: 25 or SEQ ID NO: 26 and wherein the VL comprises an amino acid sequence with at least 80%, 85%, 90%, 95%, 98%, 99%, or 100% sequence identity to SEQ ID NO: 27 or SEQ ID NO: 28.

Embodiment B5. The isolated antibody of any one of Embodiments B1-B4, wherein the isolated antibody comprises a Fab, Fab′, F(ab′)₂, or a single chain variable fragment (scFv).

Embodiment B6. The isolated antibody of any one of Embodiments B1-B5, wherein the isolated antibody is a Fab, Fab′, F(ab′)₂, or a single chain variable fragment (scFv).

Embodiment B7. The isolated antibody of Embodiments B6 or B7, wherein the isolated antibody comprises the scFv.

Embodiment B8. The isolated antibody of any one of Embodiments B1-B7, wherein the VH and VL are connected by an scFv linker sequence.

Embodiment B9. The isolated antibody of Embodiment B8, wherein the scFv linker sequence comprises an amino acid sequence according to SEQ ID NO: 29.

Embodiment B10. The isolated antibody of any one of Embodiments B5-B9, wherein the scFv comprises an amino acid sequence with at least 80%, 85%, 90%, 95%, 98%, 99%, or 100% sequence identity to SEQ ID NO: 30 or SEQ ID NO: 31.

Embodiment B11. The isolated antibody of any one of Embodiments B1-B6, wherein the isolated antibody comprises a constant domain.

Embodiment B12. The isolated antibody of Embodiment B11, wherein the constant domain comprises a human IgG4 sequence.

Embodiment B13. The isolated antibody of Embodiment B12, wherein the human IgG4 sequence comprises a S228P point mutation wherein the amino acid numbering is according to the EU index in Kabat et al.

Embodiment B14. The isolated antibody of Embodiments B12 or B13, wherein the human IgG4 sequence does not have a C-terminal lysine residue.

Embodiment B15. The isolated antibody of any one of Embodiments B11-B14, wherein the constant domain comprises a human IgG1 sequence.

Embodiment B16. The isolated antibody of any one of Embodiments B11-B15, wherein the constant domain comprises a C_{H}1, C_{H}2, C_{H}3, or a C_{L} amino acid sequence.

Embodiment B17. The isolated antibody of any one of Embodiments B1-B15, wherein the isolated antibody comprises a C_{H}1, C_{H}2, C_{H}3, and a C_{L} amino acid sequence.

Embodiment B18. The isolated antibody of any one of Embodiments B1-B17, wherein the isolated antibody comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

Embodiment B19. An isolated recombinant nucleic acid sequence encoding an amino acid sequence of the isolated antibody according to any one of Embodiments B1-B18.

Embodiment B20. A vector comprising the isolated recombinant nucleic acid sequence according to Embodiment B19.

Embodiment B21. A pharmaceutical composition comprising: (a) the isolated antibody according to any one of Embodiments B1-B18; and (b) a pharmaceutically acceptable excipient.

Embodiment B22. A method of treating a food allergy in a subject in need thereof comprising administering to the subject the isolated antibody according to any one of Embodiments B1-B18.

Embodiment B23. The method of Embodiment B22, wherein the food allergy comprises a peanut allergy.

Embodiment C1. An isolated antibody that binds to Ara h 2 that comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

Embodiment C2. The isolated antibody of Embodiment C1, wherein the VH comprises an amino acid sequence with at least 80%, 85%, 90%, 95%, 98%, 99%, or 100% sequence identity to SEQ ID NO: 42 or SEQ ID NO: 43.

Embodiment C3. The isolated antibody of Embodiments C1 or C2, wherein the VL comprises an amino acid sequence with at least 80%, 85%, 90%, 95%, 98%, 99%, or 100% sequence identity to SEQ ID NO: 44.

Embodiment C4. The isolated antibody of any one of Embodiments C1-C3, wherein the VH comprises an amino acid sequence with at least 80%, 85%, 90%, 95%, 98%, 99%, or 100% sequence identity to SEQ ID NO: 42 or SEQ ID NO: 43 and wherein the VL comprises an amino acid sequence with at least 80%, 85%, 90%, 95%, 98%, 99%, or 100% sequence identity to SEQ ID NO: 44.

Embodiment C5. The isolated antibody of any one of Embodiments C1-C4, wherein the isolated antibody comprises a Fab, Fab′, F(ab′)₂, or a single chain variable fragment (scFv).

Embodiment C6. The isolated antibody of any one of Embodiments C1-C5, wherein the isolated antibody is a Fab, Fab′, F(ab′)₂, or a single chain variable fragment (scFv).

Embodiment C7. The isolated antibody of Embodiments C5 or C6, wherein the isolated antibody comprises the scFv.

Embodiment C8. The isolated antibody of any one of Embodiments C1-C7, wherein the VH and VL are connected by an scFv linker sequence.

Embodiment C9. The isolated antibody of Embodiment C8, wherein the scFv linker sequence comprises an amino acid sequence according to SEQ ID NO: 45.

Embodiment C10. The isolated antibody of any one of Embodiments C5-C9, wherein the scFv comprises an amino acid sequence with at least 80%, 85%, 90%, 95%, 98%, 99%, or 100% sequence identity to SEQ ID NO: 46 or SEQ ID NO: 47.

Embodiment C11. The isolated antibody of any one of Embodiments C1-C6, wherein the isolated antibody comprises a constant domain.

Embodiment C12. The isolated antibody of Embodiment C11, wherein the constant domain comprises a human IgG4 sequence.

Embodiment C13. The isolated antibody of Embodiment C12, wherein the human IgG4 sequence comprises a S228P point mutation wherein the amino acid numbering is according to the EU index in Kabat et al.

Embodiment C14. The isolated antibody of Embodiments C12 or C13, wherein the human IgG4 sequence does not have a C-terminal lysine residue.

Embodiment C15. The isolated antibody of any one of Embodiments C11-C14, wherein the constant domain comprises a human IgG1 sequence.

Embodiment C16. The isolated antibody of any one of Embodiments C11-C15, wherein the constant domain comprises a C_{H}1, C_{H}2, C_{H}3, or a C_{L} amino acid sequence.

Embodiment C17. The isolated antibody of any one of Embodiments C1-C16, wherein the isolated antibody comprises a C_{H}1, C_{H}2, C_{H}3, and a C_{L} amino acid sequence.

Embodiment C18. The isolated antibody of any one of Embodiments C1-C17, wherein the isolated antibody comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

Embodiment C19. An isolated recombinant nucleic acid sequence encoding an amino acid sequence of the isolated antibody according to any one of Embodiments C1-C18.

Embodiment C20. A vector comprising the isolated recombinant nucleic acid sequence according to Embodiment C19.

Embodiment C21. A pharmaceutical composition comprising: (a) the isolated antibody according to any one of Embodiments C1-C18; and (b) a pharmaceutically acceptable excipient.

Embodiment C22. A method of treating a food allergy in a subject in need thereof comprising administering to the subject the isolated antibody according to any one of Embodiments C1-C18.

Embodiment C23. The method of Embodiment C22, wherein the food allergy comprises a peanut allergy.

Embodiment D1. An isolated antibody that comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 and Ara h 6 wherein the second binding domain comprises a VH that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24.

Embodiment D2. The isolated antibody of Embodiment D1, wherein the VH of the first binding domain comprises an amino acid sequence with at least 80%, 85%, 90%, 95%, 98%, 99%, or 100% sequence identity to SEQ ID NO: 9 or SEQ ID NO: 10.

Embodiment D3. The isolated antibody of Embodiments D1 or D2, wherein the VL of the first binding domain comprises an amino acid sequence with at least 80%, 85%, 90%, 95%, 98%, 99%, or 100% sequence identity to SEQ ID NO: 11.

Embodiment D4. The isolated antibody of any one of Embodiments D1-D3, wherein the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11.

Embodiment D5. The isolated antibody of any one of Embodiments D1-D4, wherein the first binding domain comprises a Fab, Fab′, F(ab′)₂, or a single chain variable fragment (scFv).

Embodiment D6. The isolated antibody of any one of Embodiments D1-D5, wherein the first binding domain is a Fab, Fab′, F(ab′)₂, or a single chain variable fragment (scFv).

Embodiment D7. The isolated antibody of Embodiments D5 or D6, wherein the first binding domain comprises the scFv.

Embodiment D8. The isolated antibody of any one of Embodiments D1-D7, wherein the VH of the first binding domain and VL of the first binding domain are connected by an scFv linker sequence.

Embodiment D9. The isolated antibody of Embodiment D8, wherein the scFv linker sequence of the first binding domain comprises an amino acid sequence according to SEQ ID No: 12.

Embodiment D10. The isolated antibody of any one of Embodiments D5-D9, wherein the scFv of the first binding domain comprises an amino acid sequence with at least 80%, 85%, 90%, 95%, 98%, 99%, or 100% sequence identity to SEQ ID NO: 13 or SEQ ID NO: 14.

Embodiment D11. The isolated antibody of any one of Embodiments D1-D6, wherein the first binding domain comprises a constant domain.

Embodiment D12. The isolated antibody of Embodiment D11, wherein the constant domain of the first binding domain comprises a human IgG4 sequence.

Embodiment D13. The isolated antibody of Embodiment D12, wherein the human IgG4 sequence of the first binding domain comprises a S228P point mutation wherein the amino acid numbering is according to the EU index in Kabat et al.

Embodiment D14. The isolated antibody of Embodiments D12 or D13, wherein the human IgG4 sequence of the first binding domain does not have a C-terminal lysine residue.

Embodiment D15. The isolated antibody of any one of Embodiments D11-D14, wherein the constant domain of the first binding domain comprises a human IgG1 sequence.

Embodiment D16. The isolated antibody of any one of Embodiments D11-D15, wherein the constant domain of the first binding domain comprises a C_{H}1, C_{H}2, C_{H}3, or a C_{L} amino acid sequence.

Embodiment D17. The isolated antibody of any one of Embodiments D1-D15, wherein the first binding domain comprises a C_{H}1, C_{H}2, C_{H}3, and a C_{L} amino acid sequence.

Embodiment D18. The isolated antibody of any one of Embodiments D1-D17, wherein the first binding domain comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17.

Embodiment D19. The isolated antibody of any one of Embodiments D1-D18, wherein the VH of the second binding domain comprises an amino acid sequence with at least 80%, 85%, 90%, 95%, 98%, 99%, or 100% sequence identity to SEQ ID NO: 25 or SEQ ID NO: 26.

Embodiment D20. The isolated antibody of any one of Embodiments D1-D19, wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment D21. The isolated antibody of any one of Embodiments D1-D20, wherein the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment D22. The isolated antibody of any one of Embodiments D1-D21, wherein the second binding domain comprises a Fab, Fab′, F(ab′)₂, or a single chain variable fragment (scFv).

Embodiment D23. The isolated antibody of any one of Embodiments D1-D22, wherein the second binding domain is a Fab, Fab′, F(ab′)₂, or a single chain variable fragment (scFv).

Embodiment D24. The isolated antibody of Embodiments D22 or D23, wherein the second binding domain comprises the scFv.

Embodiment D25. The isolated antibody of any one or Embodiments D1-D24, wherein the VH of the second binding domain and VL of the second binding domain are connected by an scFv linker sequence.

Embodiment D26. The isolated antibody of Embodiment D25, wherein the scFv linker sequence of the second binding domain comprises an amino acid sequence according to SEQ ID NO: 29.

Embodiment D27. The isolated antibody of any one of Embodiments D1-D26, wherein the scFv of the second binding domain comprises an amino acid sequence with at least 80%, 85%, 90%, 95%, 98%, 99%, or 100% sequence identity to SEQ ID NO: 30 or SEQ ID NO: 31.

Embodiment D28. The isolated antibody of any one of Embodiments D1-D27, wherein the second binding domain comprises a constant domain.

Embodiment D29. The isolated antibody of Embodiment D28, wherein the constant domain of the second binding domain comprises a human IgG4 sequence.

Embodiment D30. The isolated antibody of Embodiment D29, wherein the human IgG4 sequence of the second binding domain comprises a S228P point mutation wherein the amino acid numbering is according to the EU index in Kabat et al.

Embodiment D31. The isolated antibody of Embodiments D29 or D30, wherein the human IgG4 sequence of the second binding domain does not have a C-terminal lysine residue.

Embodiment D32. The isolated antibody of any one of Embodiments D28-D31, wherein the constant domain of the second binding domain comprises a human IgG1 sequence.

Embodiment D33. The isolated antibody of any one of Embodiments D28-D32, wherein the constant domain of the second binding domain comprises a C_{H}1, C_{H}2, C_{H}3, or a C_{L} amino acid sequence.

Embodiment D34. The isolated antibody of any one of Embodiments D1-D32, wherein the second binding domain comprises a C_{H}1, C_{H}2, C_{H}3, and a C_{L} amino acid sequence.

Embodiment D35. The isolated antibody of any one of Embodiments D1-D34, wherein the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

Embodiment D36. The isolated antibody of any one of Embodiments D1-D35, wherein the first binding domain comprises the scFv and the second binding domain comprises a C_{H}1, C_{H}2, C_{H}3, and a C_{L}amino acid sequence.

Embodiment D37. The isolated antibody of any one of Embodiments D1-D35, wherein the second binding domain comprises the scFv and the first binding domain comprises a C_{H}1, C_{H}2, C_{H}3, and a C_{L} amino acid sequence.

Embodiment D38. The isolated antibody of any one of Embodiments D1-D37, wherein the first binding domain and the second binding domain are connected by a linking moiety.

Embodiment D39. The isolated antibody of Embodiment D38, wherein the linking moiety is an amino acid sequence.

Embodiment D40. The isolated antibody of Embodiments D38 or D39, wherein the linking moiety is at least 4 amino acids long.

Embodiment D41. The isolated antibody of any one of Embodiments D38-D40, wherein the linking moiety is between 4-20 amino acids in length.

Embodiment D42. The isolated antibody of any one of Embodiments D38-D41, wherein the linking moiety connects the C_{H}3 amino acid sequence of the second binding domain to the scFv of the first binding domain.

Embodiment D43. The isolated antibody of any one of Embodiments D38-D42, wherein the linking moiety comprises the amino acid sequence of SEQ ID NO: 51.

Embodiment D44. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52.

Embodiment D45. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54.

Embodiment D46. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64.

Embodiment D47. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66.

Embodiment D48. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68.

Embodiment D49. The isolated antibody of any one of Embodiments D1-D43, wherein the isolated antibody comprises an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70.

Embodiment D50. An isolated recombinant nucleic acid sequence encoding an amino acid sequence of the isolated antibody according to any one of Embodiments D1-D49.

Embodiment D51. A vector comprising the isolated recombinant nucleic acid sequence according to Embodiment D50.

Embodiment D52. A pharmaceutical composition comprising: (a) the isolated antibody according to any one of Embodiments D1-D49; and (b) a pharmaceutically acceptable excipient.

Embodiment D53. A method of treating a food allergy in a subject in need thereof comprising administering to the subject the isolated antibody according to any one of Embodiments D1-D49.

Embodiment D54. The method of Embodiment D53, wherein the food allergy comprises a peanut allergy.

Embodiment EL. An isolated antibody that comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

Embodiment E2. The isolated antibody of Embodiment E1, wherein the VH of the first binding domain comprises an amino acid sequence with at least 80%, 85%, 90%, 95%, 98%, 99%, or 100% sequence identity to SEQ ID NO: 9 or SEQ ID NO: 10.

Embodiment E3. The isolated antibody of Embodiments E1 or E2, wherein the VL of the first binding domain comprises an amino acid sequence with at least 80%, 85%, 90%, 95%, 98%, 99%, or 100% sequence identity to SEQ ID NO: 11.

Embodiment E4. The isolated antibody of any one of Embodiments E1-E3, wherein the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11.

Embodiment E5. The isolated antibody of any one of Embodiments E1-E4, wherein the first binding domain comprises a Fab, Fab′, F(ab′)₂, or a single chain variable fragment (scFv).

Embodiment E6. The isolated antibody of any one of Embodiments E1-E5, wherein the first binding domain is a Fab, Fab′, F(ab′)₂, or a single chain variable fragment (scFv).

Embodiment E7. The isolated antibody of Embodiments E5 or E6, wherein the first binding domain comprises the scFv.

Embodiment E8. The isolated antibody of any one of Embodiments E1-E7, wherein the VH of the first binding domain and VL of the first binding domain are connected by an scFv linker sequence.

Embodiment E9. The isolated antibody of Embodiment E8, wherein the scFv linker sequence of the first binding domain comprises an amino acid sequence according to SEQ ID No: 12.

Embodiment E10. The isolated antibody of Embodiment E7, wherein the scFv of the first binding domain comprises an amino acid sequence with at least 80%, 85%, 90%, 95%, 98%, 99%, or 100% sequence identity to SEQ ID NO: 13 or SEQ ID NO: 14.

Embodiment E11. The isolated antibody of any one of Embodiments E1-E6, wherein the first binding domain comprises a constant domain.

Embodiment E12. The isolated antibody of Embodiment E11, wherein the constant domain of the first binding domain comprises a human IgG4 sequence.

Embodiment E13. The isolated antibody of Embodiment E12, wherein the human IgG4 sequence of the first binding domain comprises a S228P point mutation wherein the amino acid numbering is according to the EU index in Kabat et al.

Embodiment E14. The isolated antibody of Embodiments E12 or E13, wherein the human IgG4 sequence of the first binding domain does not have a C-terminal lysine residue.

Embodiment E15. The isolated antibody of any one of Embodiments E11-E14, wherein the constant domain of the first binding domain comprises a human IgG1 sequence.

Embodiment E16. The isolated antibody of any one of Embodiments E11-E15, wherein the constant domain of the first binding domain comprises a C_{H}1, C_{H}2, C_{H}3, or a C_{L} amino acid sequence.

Embodiment E17. The isolated antibody of any one of Embodiments E1-E15, wherein the first binding domain comprises a C_{H}1, C_{H}2, C_{H}3, and a C_{L} amino acid sequence.

Embodiment E18. The isolated antibody of any one of Embodiments E1-E17, wherein the first binding domain comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17.

Embodiment E19. The isolated antibody of any one of Embodiments E1-E18, wherein the VH of the second binding domain comprises an amino acid sequence with at least 80%, 85%, 90%, 95%, 98%, 99%, or 100% sequence identity to SEQ ID NO: 42 or SEQ ID NO: 43.

Embodiment E20. The isolated antibody of any one of Embodiments E1-E19, wherein the VL of the second binding domain comprises an amino acid sequence with at least 80%, 85%, 90%, 95%, 98%, 99%, or 100% sequence identity to SEQ ID NO: 44.

Embodiment E21. The isolated antibody of any one of Embodiments E1-E20, wherein the VH of the second binding domain comprises an amino acid sequence with at least 80%, 85%, 90%, 95%, 98%, 99%, or 100% sequence identity to SEQ ID NO: 42 or SEQ ID NO: 43 and wherein the VL of the second binding domain comprises an amino acid sequence with at least 80%, 85%, 90%, 95%, 98%, 99%, or 100% sequence identity to SEQ ID NO: 44.

Embodiment E22. The isolated antibody of any one of Embodiments E1-E21, wherein the second binding domain comprises a Fab, Fab′, F(ab′)₂, or a single chain variable fragment (scFv).

Embodiment E23. The isolated antibody of any one of Embodiments E1-E22, wherein the second binding domain is a Fab, Fab′, F(ab′)₂, or a single chain variable fragment (scFv).

Embodiment E24. The isolated antibody of Embodiments E22 or E23, wherein the second binding domain comprises the scFv.

Embodiment E25. The isolated antibody of any one of Embodiments E1-E24, wherein the VH of the second binding domain and VL of the second binding domain are connected by an scFv linker sequence.

Embodiment E26. The isolated antibody of Embodiment E25, wherein the scFv linker sequence of the second binding domain comprises an amino acid sequence according to SEQ ID NO: 45.

Embodiment E27. The isolated antibody of any one of Embodiments E1-E26, wherein the scFv of the second binding domain comprises an amino acid sequence with at least 80%, 85%, 90%, 95%, 98%, 99%, or 100% sequence identity to SEQ ID NO: 46 or SEQ ID NO: 47.

Embodiment E28. The isolated antibody of any one of Embodiments E1-E23, wherein the second binding domain comprises a constant domain.

Embodiment E29. The isolated antibody of Embodiment E28, wherein the constant domain of the second binding domain comprises a human IgG4 sequence.

Embodiment E30. The isolated antibody of Embodiment E29, wherein the human IgG4 sequence of the second binding domain comprises a S228P point mutation wherein the amino acid numbering is according to the EU index in Kabat et al.

Embodiment E31. The isolated antibody of Embodiment E28 or E29, wherein the human IgG4 sequence of the second binding domain does not have a C-terminal lysine residue.

Embodiment E32. The isolated antibody of any one of Embodiments E28-E31, wherein the constant domain of the second binding domain comprises a human IgG1 sequence.

Embodiment E33. The isolated antibody of any one of Embodiments E28-E32, wherein the constant domain of the second binding domain comprises a C_{H}1, C_{H}2, C_{H}3, or a C_{L} amino acid sequence.

Embodiment E34. The isolated antibody of any one of Embodiments E1-E32, wherein the second binding domain comprises a C_{H}1, C_{H}2, C_{H}3, and a C_{L} amino acid sequence.

Embodiment E35. The isolated antibody of any one of Embodiments E1-E34, wherein the second binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

Embodiment E36. The isolated antibody of any one of embodiments E1-E35, wherein the first binding domain comprises the scFv and the second binding domain comprises a C_{H}1, C_{H}2, C_{H}3, and a C_{L}amino acid sequence.

Embodiment E37. The isolated antibody of any one of embodiments E1-E35, wherein the second binding domain comprises the scFv and the first binding domain comprises a C_{H}1, C_{H}2, C_{H}3, and a C_{L} amino acid sequence.

Embodiment E38. The isolated antibody of any one of Embodiments E1-E37, wherein the first binding domain and the second binding domain are connected by a linking moiety.

Embodiment E39. The isolated antibody of Embodiment E38, wherein the linking moiety is an amino acid sequence.

Embodiment E40. The isolated antibody of Embodiment E38 or E39, wherein the linking moiety is at least 4 amino acids long.

Embodiment E41. The isolated antibody of any one of Embodiments E38-E41, wherein the linking moiety is between 4-20 amino acids in length.

Embodiment E42. The isolated antibody of any one of Embodiments E38-E41, wherein the linking moiety connects the C_{H}3 amino acid sequence of the second binding domain to the scFv of the first binding domain.

Embodiment E43. The isolated antibody of any one Embodiments E38-E42, wherein the linking moiety comprises the amino acid sequence of SEQ ID NO: 51.

Embodiment E44. The isolated antibody of any one of Embodiments E1-E43, wherein the isolated antibody comprises an amino acid sequence with at least 80%, 85%, 90%, 95%, 98%, 99%, or 100% sequence identity to SEQ ID NO: 57 and an amino acid sequence with at least 80%, 85%, 90%, 95%, 98%, 99%, or 100% sequence identity to SEQ ID NO: 56.

Embodiment E45. The isolated antibody of any one of Embodiments E1-E43, wherein the isolated antibody comprises an amino acid sequence with at least 80%, 85%, 90%, 95%, 98%, 99%, or 100% sequence identity to SEQ ID NO: 59 and an amino acid sequence with at least 80%, 85%, 90%, 95%, 98%, 99%, or 100% sequence identity to SEQ ID NO: 58.

Embodiment E46. An isolated recombinant nucleic acid sequence encoding an amino acid sequence of the isolated antibody according to any one of Embodiments E1-E45.

Embodiment E47. A vector comprising the isolated recombinant nucleic acid sequence according to Embodiment E46.

Embodiment E48. A pharmaceutical composition comprising: (a) the isolated antibody according to any one of Embodiments E1-E45; and (b) a pharmaceutically acceptable excipient.

Embodiment E49. A method of treating a food allergy in a subject in need thereof comprising administering to the subject the isolated antibody according to any one of Embodiments E1-E45.

Embodiment E50. The method of Embodiment E49, wherein the food allergy comprises a peanut allergy.

Embodiment F1. An isolated antibody that comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, wherein the isolated antibody comprises a second binding domain that binds to Ara h 2 wherein the second binding domain comprises a (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the isolated antibody comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

Embodiment F2. The isolated antibody of Embodiment F1, wherein the VH of the first binding domain comprises an amino acid sequence with at least 80%, 85%, 90%, 95%, 98%, 99%, or 100% sequence identity to SEQ ID NO: 25 or SEQ ID NO: 26.

Embodiment F3. The isolated antibody of Embodiment F1 or F2, wherein the VL of the first binding domain comprises an amino acid sequence with at least 80%, 85%, 90%, 95%, 98%, 99%, or 100% sequence identity to SEQ ID NO: 27 or SEQ ID NO: 28.

Embodiment F4. The isolated antibody of any one of Embodiments F1-F3, wherein the VH of the first binding domain comprises an amino acid sequence with at least 80%, 85%, 90%, 95%, 98%, 99%, or 100% sequence identity to SEQ ID NO: 25 or SEQ ID NO: 26 and wherein the VL of the first binding domain comprises an amino acid sequence with at least 80%, 85%, 90%, 95%, 98%, 99%, or 100% sequence identity to SEQ ID NO: 27 or SEQ ID NO: 28.

Embodiment F5. The isolated antibody of any one of Embodiments F1-F4, wherein the first binding domain comprises a Fab, Fab′, F(ab′)₂, or a single chain variable fragment (scFv).

Embodiment F6. The isolated antibody of any one of Embodiments F1-F5, wherein the first binding domain is a Fab, Fab′, F(ab′)₂, or a single chain variable fragment (scFv).

Embodiment F7. The isolated antibody of Embodiment F5 or F6, wherein the first binding domain comprises the scFv.

Embodiment F8. The isolated antibody of any one of Embodiments F1-F7, wherein the VH of the first binding domain and VL of the first binding domain are connected by an scFv linker sequence.

Embodiment F9. The isolated antibody of Embodiment F8, wherein the scFv linker sequence of the first binding domain comprises an amino acid sequence according to SEQ ID NO: 29.

Embodiment F10. The isolated antibody of any one of Embodiments F1-F9, wherein the scFv of the first binding domain comprises an amino acid sequence with at least 80%, 85%, 90%, 95%, 98%, 99%, or 100% sequence identity to SEQ ID NO: 30 or SEQ ID NO: 31.

Embodiment F11. The isolated antibody of any one of Embodiments F1-F6, wherein the first binding domain comprises a constant domain.

Embodiment F12. The isolated antibody of Embodiment F11, wherein the constant domain of the first binding domain comprises a human IgG4 sequence.

Embodiment F13. The isolated antibody of Embodiment F12, wherein the human IgG4 sequence of the first binding domain comprises a S228P point mutation wherein the amino acid numbering is according to the EU index in Kabat et al.

Embodiment F14. The isolated antibody of Embodiment F12 or F13, wherein the human IgG4 sequence of the first binding domain does not have a C-terminal lysine residue.

Embodiment F15. The isolated antibody of any one of Embodiments F11-F14, wherein the constant domain of the first binding domain comprises a human IgG1 sequence.

Embodiment F16. The isolated antibody of any one of Embodiments F11-F15, wherein the constant domain of the first binding domain comprises a C_{H}1, C_{H}2, C_{H}3, or a C_{L} amino acid sequence.

Embodiment F17. The isolated antibody of any one of Embodiments F1-F15, wherein the first binding domain comprises a C_{H}1, C_{H}2, C_{H}3, and a C_{L} amino acid sequence.

Embodiment F18. The isolated antibody of any one of Embodiments F1-F17, wherein the first binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

Embodiment F19. The isolated antibody of any one of Embodiments F1-F18, wherein the VH of the second binding domain comprises an amino acid sequence with at least 80%, 85%, 90%, 95%, 98%, 99%, or 100% sequence identity to SEQ ID NO: 42 or SEQ ID NO: 43.

Embodiment F20. The isolated antibody of any one of Embodiments F1-F19, wherein the VL of the second binding domain comprises an amino acid sequence with at least 80%, 85%, 90%, 95%, 98%, 99%, or 100% sequence identity to SEQ ID NO: 44.

Embodiment F21. The isolated antibody of any one of Embodiments F1-F20, wherein the VH of the second binding domain comprises an amino acid sequence with at least 80%, 85%, 90%, 95%, 98%, 99%, or 100% sequence identity to SEQ ID NO: 42 or SEQ ID NO: 43 and wherein the VL of the second binding domain comprises an amino acid sequence with at least 80%, 85%, 90%, 95%, 98%, 99%, or 100% sequence identity to SEQ ID NO: 44.

Embodiment F22. The isolated antibody of any one of Embodiments F1-F21, wherein the second binding domain comprises a Fab, Fab′, F(ab′)₂, or a single chain variable fragment (scFv).

Embodiment F23. The isolated antibody of any one of Embodiments F1-F22, wherein the second binding domain is a Fab, Fab′, F(ab′)₂, or a single chain variable fragment (scFv).

Embodiment F24. The isolated antibody of Embodiment F22 or F23, wherein the second binding domain comprises the scFv.

Embodiment F25. The isolated antibody of any one of Embodiments F1-F24, wherein the VH of the second binding domain and VL of the second binding domain are connected by an scFv linker sequence.

Embodiment F26. The isolated antibody of Embodiment F25, wherein the scFv linker sequence of the second binding domain comprises an amino acid sequence according to SEQ ID NO: 45.

Embodiment F27. The isolated antibody of any one of Embodiments F1-F26, wherein the scFv of the second binding domain comprises an amino acid sequence with at least 80%, 85%, 90%, 95%, 98%, 99%, or 100% sequence identity to SEQ ID NO: 46 or SEQ ID NO: 47.

Embodiment F28. The isolated antibody of any one of Embodiments F1-F23, wherein the second binding domain comprises a constant domain.

Embodiment F29. The isolated antibody of Embodiment F28, wherein the constant domain of the second binding domain comprises a human IgG4 sequence.

Embodiment F30. The isolated antibody of Embodiment F29, wherein the human IgG4 sequence of the second binding domain comprises a S228P point mutation wherein the amino acid numbering is according to the EU index in Kabat et al.

Embodiment F31. The isolated antibody of Embodiment F29 or F30, wherein the human IgG4 sequence of the second binding domain does not have a C-terminal lysine residue.

Embodiment F32. The isolated antibody of any one of Embodiments F28-F31, wherein the constant domain of the second binding domain comprises a human IgG1 sequence.

Embodiment F33. The isolated antibody of any one of Embodiments F28-F32, wherein the constant domain of the second binding domain comprises a C_{H}1, C_{H}2, C_{H}3, or a C_{L} amino acid sequence.

Embodiment F34. The isolated antibody of any one of Embodiments F1-F32, wherein the second binding domain comprises a C_{H}1, C_{H}2, C_{H}3, and a C_{L} amino acid sequence.

Embodiment F35. The isolated antibody of any one of Embodiments F1-F34, wherein the second binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

Embodiment F36. The isolated antibody of any one of Embodiments F1-F35, wherein the first binding domain comprises the scFv and the second binding domain comprises a C_{H}1, C_{H}2, C_{H}3, and a C_{L}amino acid sequence.

Embodiment F37. The isolated antibody of any one of Embodiments F1-F35, wherein the second binding domain comprises the scFv and the first binding domain comprises a C_{H}1, C_{H}2, C_{H}3, and a C_{L} amino acid sequence.

Embodiment F38. The isolated antibody of any one of Embodiments F1-F37, wherein the first binding domain and the second binding domain are connected by a linking moiety.

Embodiment F39. The isolated antibody of Embodiment F38, wherein the linking moiety is an amino acid sequence.

Embodiment F40. The isolated antibody of Embodiment F38 or F39, wherein the linking moiety is at least 4 amino acids long.

Embodiment F41. The isolated antibody of any one of Embodiments F38-F40, wherein the linking moiety is between 4-20 amino acids in length.

Embodiment F42. The isolated antibody of any one of Embodiments F38-F41, wherein the linking moiety connects the C_{H}3 amino acid sequence of the second binding domain to the scFv of the first binding domain.

Embodiment F43. The isolated antibody of any one of Embodiments F38-F42, wherein the linking moiety comprises the amino acid sequence of SEQ ID NO: 51.

Embodiment F44. The isolated antibody of any one of Embodiments F1-F43, wherein the isolated antibody comprises an amino acid sequence with at least 80%, 85%, 90%, 95%, 98%, 99%, or 100% sequence identity to SEQ ID NO: 61 and an amino acid sequence with at least 80%, 85%, 90%, 95%, 98%, 99%, or 100% sequence identity to SEQ ID NO: 60.

Embodiment F45. The isolated antibody of any one of Embodiments F1-F43, wherein the isolated antibody comprises an amino acid sequence with at least 80%, 85%, 90%, 95%, 98%, 99%, or 100% sequence identity to SEQ ID NO: 63 and an amino acid sequence with at least 80%, 85%, 90%, 95%, 98%, 99%, or 100% sequence identity to SEQ ID NO: 62.

Embodiment F46. An isolated recombinant nucleic acid sequence encoding an amino acid sequence of the isolated antibody according to any one of Embodiments F1-F45.

Embodiment F47. A vector comprising the isolated recombinant nucleic acid sequence according to Embodiment F46.

Embodiment F48. A pharmaceutical composition comprising: (a) the isolated antibody according to any one of Embodiments F1-F47; and (b) a pharmaceutically acceptable excipient.

Embodiment F49. A method of treating a food allergy in a subject in need thereof comprising administering to the subject the isolated antibody according to any one of Embodiment F1-F47.

Embodiment F50. The method of Embodiment F49, wherein the food allergy comprises a peanut allergy.

Embodiment G1. A composition that comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

Embodiment G2. The composition of Embodiment G1, wherein the VH of the first binding domain comprises an amino acid sequence with at least 80%, 85%, 90%, 95%, 98%, 99%, or 100% sequence identity to SEQ ID NO: 9 or SEQ ID NO: 10.

Embodiment G3. The composition of Embodiment G1 or G2, wherein the VL of the first binding domain comprises an amino acid sequence with at least 80%, 85%, 90%, 95%, 98%, 99%, or 100% sequence identity to SEQ ID NO: 11.

Embodiment G4. The composition of any one of Embodiments G1-G3, wherein the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11.

Embodiment G5. The composition of any one of Embodiments G1-G4, wherein the first binding domain comprises a Fab, Fab′, F(ab′)₂, or a single chain variable fragment (scFv).

Embodiment G6. The composition of any one of Embodiments G1-G5, wherein the first binding domain is a Fab, Fab′, F(ab′)₂, or a single chain variable fragment (scFv).

Embodiment G7. The composition of Embodiment G5 or G6, wherein the first binding domain comprises the scFv.

Embodiment G8. The composition of any one of Embodiments G1-G7, wherein the VH of the first binding domain and VL of the first binding domain are connected by an scFv linker sequence.

Embodiment G9. The composition of Embodiment G8, wherein the scFv linker sequence of the first binding domain comprises an amino acid sequence according to SEQ ID No: 12.

Embodiment G10. The composition of any one of Embodiments G5-G9, wherein the scFv of the first binding domain comprises an amino acid sequence with at least 80%, 85%, 90%, 95%, 98%, 99%, or 100% sequence identity to SEQ ID NO: 13 or SEQ ID NO: 14.

Embodiment G11. The composition of any one of Embodiments G1-G6, wherein the first binding domain comprises a constant domain.

Embodiment G12. The composition of Embodiment G11, wherein the constant domain of the first binding domain comprises a human IgG4 sequence.

Embodiment G13. The composition of Embodiment G12, wherein the human IgG4 sequence of the first binding domain comprises a S228P point mutation wherein the amino acid numbering is according to the EU index in Kabat et al.

Embodiment G14. The composition of Embodiment G12 or G13, wherein the human IgG4 sequence of the first binding domain does not have a C-terminal lysine residue.

Embodiment G15. The composition of any one of Embodiments G11-G14, wherein the constant domain of the first binding domain comprises a human IgG1 sequence.

Embodiment G16. The composition of any one of Embodiments G11-G15, wherein the constant domain of the first binding domain comprises a C_{H}1, C_{H}2, C_{H}3, or a C_{L} amino acid sequence.

Embodiment G17. The composition of any one of Embodiments G1-G15, wherein the first binding domain comprises a C_{H}1, C_{H}2, C_{H}3, and a C_{L} amino acid sequence.

Embodiment G18. The composition of any one of Embodiments G1-G17, wherein the first binding domain comprises an amino acid sequence with at least 80%, 85%, 90%, 95%, 98%, 99%, or 100% sequence identity to SEQ ID NO:15, SEQ ID NO:16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17.

Embodiment G19. The composition of any one of Embodiments G1-G18, wherein the VH of the second binding domain comprises an amino acid sequence with at least 80%, 85%, 90%, 95%, 98%, 99%, or 100% sequence identity to SEQ ID NO: 25 or SEQ ID NO: 26.

Embodiment G20. The composition of any one of Embodiments G1-G19, wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment G21. The composition of any one of Embodiments G1-G20, wherein the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment G22. The composition of any one of Embodiments G1-G21, wherein the second binding domain comprises a Fab, Fab′, F(ab′)₂, or a single chain variable fragment (scFv).

Embodiment G23. The composition of any one of Embodiments G1-G22, wherein the second binding domain is a Fab, Fab′, F(ab′)₂, or a single chain variable fragment (scFv).

Embodiment G24. The composition of Embodiment G22 or G23, wherein the second binding domain comprises the scFv.

Embodiment G25. The composition of any one of Embodiments G1-G24, wherein the VH of the second binding domain and VL of the second binding domain are connected by an scFv linker sequence.

Embodiment G26. The composition of Embodiment G25, wherein the scFv linker sequence of the second binding domain comprises an amino acid sequence according to SEQ ID NO: 29.

Embodiment G27. The composition of any one of Embodiments G22-G26, wherein the scFv of the second binding domain comprises an amino acid sequence with at least 80%, 85%, 90%, 95%, 98%, 99%, or 100% sequence identity to SEQ ID NO: 30 or SEQ ID NO: 31.

Embodiment G28. The composition of any one of Embodiments G1-G23, wherein the second binding domain comprises a constant domain.

Embodiment G29. The composition of Embodiment G28, wherein the constant domain of the second binding domain comprises a human IgG4 sequence.

Embodiment G30. The composition of Embodiment G29, wherein the human IgG4 sequence of the second binding domain comprises a S228P point mutation wherein the amino acid numbering is according to the EU index in Kabat et al.

Embodiment G31. The composition of Embodiment G29 or G30, wherein the human IgG4 sequence of the second binding domain does not have a C-terminal lysine residue.

Embodiment G32. The composition of any one of Embodiments G28-G31, wherein the constant domain of the second binding domain comprises a human IgG1 sequence.

Embodiment G33. The composition of any one of Embodiments G28-G32, wherein the constant domain of the second binding domain comprises a C_{H}1, C_{H}2, C_{H}3, or a C_{L} amino acid sequence.

Embodiment G34. The composition of any one of Embodiments G1-G32, wherein the second binding domain comprises a C_{H}1, C_{H}2, C_{H}3, and a C_{L} amino acid sequence.

Embodiment G35. The composition of any one of Embodiments G1-G34, wherein the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

Embodiment G36. The composition of any one of Embodiments G1-G35, wherein the VH of the third binding domain comprises an amino acid sequence with at least 80%, 85%, 90%, 95%, 98%, 99%, or 100% sequence identity to SEQ ID NO: 42 or SEQ ID NO: 43.

Embodiment G37. The composition of any one of Embodiments G1-G36, wherein the VL of the third binding domain comprises an amino acid sequence with at least 80%, 85%, 90%, 95%, 98%, 99%, or 100% sequence identity to SEQ ID NO: 44.

Embodiment G38. The composition of any one of Embodiments G1-G37, wherein the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44.

Embodiment G39. The composition of any one of Embodiments G1-G38, wherein the third binding domain comprises a Fab, Fab′, F(ab′)₂, or a single chain variable fragment (scFv).

Embodiment G40. The composition of any one of Embodiments G1-G39, wherein the third binding domain is a Fab, Fab′, F(ab′)₂, or a single chain variable fragment (scFv).

Embodiment G41. The composition of Embodiment G39 or G40, wherein the third binding domain comprises the scFv.

Embodiment G42. The composition of any one of Embodiments G1-G41, wherein the VH of the third binding domain and VL of the third binding domain are connected by an scFv linker sequence.

Embodiment G43. The composition of Embodiment G42, wherein the scFv linker sequence of the third binding domain comprises an amino acid sequence according to SEQ ID NO: 45.

Embodiment G44. The composition of any one of Embodiments G39-G43, wherein the scFv of the third binding domain comprises an amino acid sequence with at least 80%, 85%, 90%, 95%, 98%, 99%, or 100% sequence identity to SEQ ID NO: 46 or SEQ ID NO: 47.

Embodiment G45. The composition of any one of Embodiments G1-G40, wherein the third binding domain comprises a constant domain.

Embodiment G46. The composition of Embodiment G45, wherein the constant domain of the third binding domain comprises a human IgG4 sequence.

Embodiment G47. The composition of Embodiment G46, wherein the human IgG4 sequence of the third binding domain comprises a S228P point mutation wherein the amino acid numbering is according to the EU index in Kabat et al.

Embodiment G48. The composition of Embodiment G46 or G47, wherein the human IgG4 sequence of the third binding domain does not have a C-terminal lysine residue.

Embodiment G49. The composition of any one of Embodiments G45-G48, wherein the constant domain of the third binding domain comprises a human IgG1 sequence.

Embodiment G50. The composition of any one of Embodiments G45-G49, wherein the constant domain of the third binding domain comprises a C_{H}1, C_{H}2, C_{H}3, or a C_{L} amino acid sequence.

Embodiment G51. The composition of any one of Embodiments G1-G49, wherein the third binding domain comprises a C_{H}1, C_{H}2, C_{H}3, and a C_{L} amino acid sequence.

Embodiment G52. The composition of any one of Embodiments G1-G51, wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

Embodiment G53. The composition of any one of Embodiments G1-G52, wherein the first binding domain, second binding domain, and the third binding domain are connected within the same molecule.

Embodiment G54. The composition of any one of Embodiments G1-G52, wherein the first binding domain, second binding domain, and the third binding domain are separate molecules.

Embodiment G55. The composition of any one of Embodiments G1-G52, wherein the first binding domain and second binding domain are within the same molecule and the third binding domain is a separate molecule from the molecule with the first binding domain and the second binding domain.

Embodiment G56. The composition of any one of Embodiments G1-G52, wherein the first binding domain and third binding domain are within the same molecule and the second binding domain is a separate molecule from the molecule with the first binding domain and the third binding domain.

Embodiment G57. The composition of any one of Embodiments G1-G52, wherein the second binding domain and third binding domain are within the same molecule and the first binding domain is a separate molecule from the molecule with the second binding domain and the third binding domain.

Embodiment G58. The composition of any one of Embodiments G5-G57, wherein the first binding domain comprises the scFv and the second binding domain comprises a C_{H}1, C_{H}2, C_{H}3, and a C_{L} amino acid sequence.

Embodiment G59. The composition of any one of Embodiments G22-G58, wherein the second binding domain comprises the scFv and the first binding domain comprises a C_{H}1, C_{H}2, C_{H}3, and a C_{L} amino acid sequence.

Embodiment G60. The composition of any one of Embodiments G1-G59, wherein the first binding domain and the second binding domain are connected by a linking moiety.

Embodiment G61. The composition of Embodiment G60, wherein the linking moiety is an amino acid sequence.

Embodiment G62. The composition of Embodiment G60 or G61, wherein the linking moiety is at least 4 amino acids long.

Embodiment G63. The composition of any one of Embodiments G60-G62, wherein the linking moiety is between 4-20 amino acids in length.

Embodiment G64. The composition of any one of Embodiments G60-G63, wherein the linking moiety connects the C_{H}3 amino acid sequence of the second binding domain to the scFv of the first binding domain.

Embodiment G65. The composition of any one of Embodiments G60-G64, wherein the linking moiety comprises the amino acid sequence of SEQ ID NO: 51.

Embodiment G66. The composition of any one of Embodiments GI-G65, wherein the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G67. The composition of any one of Embodiments GI-G65, wherein the first and second binding domains comprise an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G68. The composition of any one of Embodiments G1-G65, wherein the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G69. The composition of any one of Embodiments G1-G65, wherein the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G70. The composition of any one of Embodiments G1-G65, wherein the first and second binding domains comprise an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G71. The composition of any one of Embodiments G1-G65, wherein the first and second binding domains comprise an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment G72. The composition of any one of Embodiments G1-G71, wherein the composition comprises a pharmaceutically acceptable excipient.

Embodiment G73. An isolated recombinant nucleic acid sequence encoding an amino acid sequence of the composition according to any one of Embodiments G1-G71.

Embodiment G74. A vector comprising the isolated recombinant nucleic acid sequence according to Embodiment G73.

Embodiment G75. A kit that comprises at least one of: (a) the composition of any one of Embodiments GI-G72; (b) the vector of Embodiment G74; or (b) the nucleic acid molecule of Embodiment G73.

Embodiment G76. A method of treating a food allergy in a subject in need thereof comprising administering to the subject the composition according to any one of Embodiments G1-G72.

Embodiment G77. The method of Embodiment G76, wherein the food allergy comprises a peanut allergy.

Embodiment H1. A composition that comprises a first molecule that comprises a first binding domain that binds to Ara h 2 and Ara h 6 and a second binding domain that binds to Ara h 2 and Ara h 6, wherein the first binding domain comprises a scFv and the second binding domain comprises a heavy chain variable domain, a light chain variable domain, and a constant domain, and the scFv of the first binding domain is connected to the constant domain of the second binding domain by a linking moiety, and the composition comprises a second molecule that comprises a third binding domain that binds to Ara h 2.

Embodiment H2. The composition of Embodiment H1, wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

Embodiment H3. The composition of Embodiment H2, wherein the VH of the first binding domain comprises an amino acid sequence with at least 80%, 85%, 90%, 95%, 98%, 99%, or 100% sequence identity to SEQ ID NO: 9 or SEQ ID NO: 10.

Embodiment H4. The composition of any one of Embodiment H2 or H3, wherein the VL of the first binding domain comprises an amino acid sequence with at least 80%, 85%, 90%, 95%, 98%, 99%, or 100% sequence identity to SEQ ID NO: 11.

Embodiment H5. The composition of any one of Embodiments H2-H4, wherein the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11.

Embodiment H6. The composition of any one of Embodiments H2-H5, wherein the VH of the first binding domain and VL of the first binding domain are connected by an scFv linker sequence.

Embodiment H7. The composition of Embodiment H6, wherein the scFv linker sequence of the first binding domain comprises an amino acid sequence according to SEQ ID No: 12.

Embodiment H8. The composition of any one of Embodiments H1-H7, wherein the scFv of the first binding domain comprises an amino acid sequence with at least 80%, 85%, 90%, 95%, 98%, 99%, or 100% sequence identity to SEQ ID NO: 13 or SEQ ID NO: 14.

Embodiment H9. The composition of any one of Embodiments H2-H8, wherein the VH of the second binding domain comprises an amino acid sequence with at least 80%, 85%, 90%, 95%, 98%, 99%, or 100% sequence identity to SEQ ID NO: 25 or SEQ ID NO: 26.

Embodiment H10. The composition of any one of Embodiments H2-H9, wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment H11. The composition of any one of Embodiments H1-H10, wherein the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28.

Embodiment H12. The composition of any one of Embodiments H1-H11, wherein the second binding domain comprises a Fab, Fab′, or a F(ab′)₂

Embodiment H13. The composition of any one of Embodiments H1-H12, wherein the second binding domain is a Fab, Fab′, or a F(ab′)₂

Embodiment H14. The composition of any one of Embodiments H1-H13, wherein the constant domain of the second binding domain comprises a human IgG4 sequence.

Embodiment H15. The composition of Embodiment H14, wherein the human IgG4 sequence of the second binding domain comprises a S228P point mutation wherein the amino acid numbering is according to the EU index in Kabat et al.

Embodiment H16. The composition of Embodiment H14 or H15, wherein the human IgG4 sequence of the second binding domain does not have a C-terminal lysine residue.

Embodiment H17. The composition of any one of Embodiments H1-H16, wherein the constant domain of the second binding domain comprises a human IgG1 sequence.

Embodiment H18. The composition of any one of Embodiments H1-H17, wherein the constant domain of the second binding domain comprises a C_{H}1, C_{H}2, C_{H}3, or a C_{L} amino acid sequence.

Embodiment H19. The composition of any one of Embodiments H1-H18, wherein the second binding domain comprises a C_{H}1, C_{H}2, C_{H}3, and a C_{L} amino acid sequence.

Embodiment H20. The composition of any one of Embodiments H1-H19, wherein the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

Embodiment H21. The composition of any one of Embodiments H2-H20, wherein the VH of the third binding domain comprises an amino acid sequence with at least 80%, 85%, 90%, 95%, 98%, 99%, or 100% sequence identity to SEQ ID NO: 42 or SEQ ID NO: 43.

Embodiment H22. The composition of any one of Embodiments H2-H21, wherein the VL of the third binding domain comprises an amino acid sequence with at least 80%, 85%, 90%, 95%, 98%, 99%, or 100% sequence identity to SEQ ID NO: 44.

Embodiment H23. The composition of any one of Embodiments H2-H22, wherein the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44.

Embodiment H24. The composition of any one of Embodiments H1-H23, wherein the third binding domain comprises a Fab, Fab′, F(ab′)₂, or a single chain variable fragment (scFv).

Embodiment H25. The composition of any one of Embodiments H1-H24, wherein the third binding domain is a Fab, Fab′, F(ab′)₂, or a single chain variable fragment (scFv).

Embodiment H26. The composition of Embodiments H24 or H25, wherein the third binding domain comprises the scFv.

Embodiment H27. The composition of any one of Embodiments H2-H26, wherein the VH of the third binding domain and VL of the third binding domain are connected by an scFv linker sequence.

Embodiment H28. The composition of Embodiment H27, wherein the scFv linker sequence of the third binding domain comprises an amino acid sequence according to SEQ ID NO: 45.

Embodiment H29. The composition of Embodiment H26, wherein the scFv of the third binding domain comprises an amino acid sequence with at least 80%, 85%, 90%, 95%, 98%, 99%, or 100% sequence identity to SEQ ID NO: 46 or SEQ ID NO: 47.

Embodiment H30. The composition of any one of Embodiments H1-H25, wherein the third binding domain comprises a constant domain.

Embodiment H31. The composition of Embodiment H30, wherein the constant domain of the third binding domain comprises a human IgG4 sequence.

Embodiment H32. The composition of Embodiment H31, wherein the human IgG4 sequence of the third binding domain comprises a S228P point mutation wherein the amino acid numbering is according to the EU index in Kabat et al.

Embodiment H33. The composition of Embodiment H31 or H32, wherein the human IgG4 sequence of the third binding domain does not have a C-terminal lysine residue.

Embodiment H34. The composition of any one of Embodiments H30-H33, wherein the constant domain of the third binding domain comprises a human IgG1 sequence.

Embodiment H35. The composition of any one of Embodiments H30-H34, wherein the constant domain of the third binding domain comprises a C_{H}1, C_{H}2, C_{H}3, or a C_{L} amino acid sequence.

Embodiment H36. The composition of any one of Embodiments H1-H35, wherein the third binding domain comprises a C_{H}1, C_{H}2, C_{H}3, and a C_{L} amino acid sequence.

Embodiment H37. The composition of any one of Embodiments H1-H36, wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

Embodiment H38. The composition of any one of Embodiments H1-H37, wherein the first binding domain and the second binding domain are connected by a linking moiety.

Embodiment H39. The composition of Embodiment H38, wherein the linking moiety is an amino acid sequence.

Embodiment H40. The composition of Embodiment H38 or H39, wherein the linking moiety is at least 4 amino acids long.

Embodiment H41. The composition of any one of Embodiments H38-H40, wherein the linking moiety is between 4-20 amino acids in length.

Embodiment H42. The composition of any one of Embodiments H38-H41, wherein the linking moiety connects the C_{H}3 amino acid sequence of the second binding domain to the scFv of the first binding domain.

Embodiment H43. The composition of any one of Embodiments H38-H42, wherein the linking moiety comprises the amino acid sequence of SEQ ID NO: 51.

Embodiment H44. The composition of any one of Embodiments H1-H43, wherein the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment H45. The composition of any one of Embodiments H1-H43, wherein the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment H46. The composition of any one of Embodiments H1-H43, wherein the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

Embodiment H47. The composition of any one of Embodiments H1-H46, wherein the composition comprises a pharmaceutically acceptable excipient.

Embodiment H48. An isolated recombinant nucleic acid sequence encoding an amino acid sequence of the isolated antibody according to any one of Embodiments H1-H46.

Embodiment H49. A vector comprising the isolated recombinant nucleic acid sequence according to Embodiment H48.

Embodiment H50. A kit that comprises at least one of: (a) the composition of any one of Embodiments H1-H47; (b) the vector of Embodiment H49; or (b) the nucleic acid molecule of Embodiment H48.

Embodiment H51. A method of treating a food allergy in a subject in need thereof comprising administering to the subject the composition according to any one of Embodiments H1-H47.

Embodiment H52. The method of Embodiment H51, wherein the food allergy comprises a peanut allergy.

### EXAMPLES

### Example 1. Discovery of IgE Monoclonal Antibodies from Peanut Allergic Individuals

Blood was collected from dozens of highly peanut allergic individuals (peanut specific IgE >15kU/A) across the United States under informed consent in an Institutional Review Board (IRB) approved observational study. The SEQ SIFTER™ platform for single cell sorting and transcriptomics was then applied to capture extremely rare circulating IgE-producing B cells in an unbiased fashion (Croote et al. “High affinity allergen-specific human antibodies cloned from single IgE B cell transcriptomes,” Science 362.6420 (2018): 1306-1309). High-throughput cloud-based bioinformatics pipelines were then used to reassemble full-length, paired, heavy and light chain sequences that comprise monoclonal IgE antibodies thus providing insight into the IgE repertoires of allergic individuals.

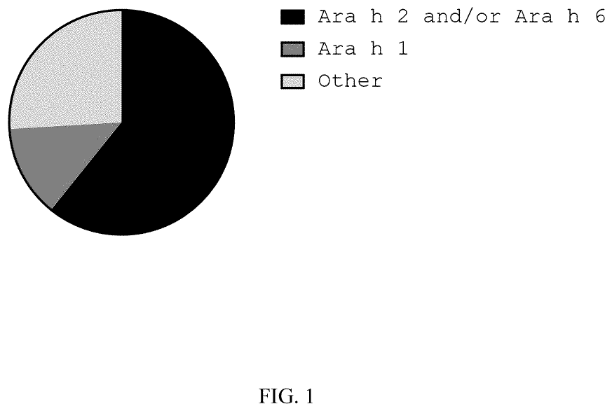

Discovered IgE variable region sequences from peanut allergic individuals were expressed recombinantly with IgG4, rather than IgE, constant regions. In doing so, monoclonal antibodies (mAbs) were generated with the specificities of the original IgE antibodies that now block, rather than initiate, the allergic cascade. As this discovery process is unbiased with regards to the specificity of the IgE antibody produced by circulating B cells, the distribution of Ara h allergen specificities naturally reflects the repertoire of circulating IgE-producing B cells. It was found that a majority of the peanut specific antibodies are specific to Ara h 2 and/or the homologous allergen Ara h 6 (FIG. 1). Notably, while there were several mAbs discovered against Ara h 1, these originated predominantly from a single individual and were lower affinity relative to mAbs specific to Ara h 2 and Ara h 6, with only 13% of Ara h 1-specific mAbs in the sub-nanomolar range. This led to a focus on Ara h 2. FIG. 1 illustrates the specificity distribution of 158 mAbs that were discovered in an unbiased fashion from 27 peanut allergic individuals. “Other” includes mAbs specific to Ara h 3, Ara h 8, and Ara h 9, as well as mAbs that had no detectable binding to Ara h 1, Ara h 2, Ara h 3, Ara h 6, Ara h 8, or Ara h 9 on ImmunoCAP.

### Example 2. Measurement of Monoclonal Antibody Affinity and Epitope Binning

The Carterra LSA, an instrument that utilizes surface plasmon resonance (SPR), was used for both antibody affinity measurement and epitope binning, a pairwise competition assay that groups antibodies by the epitope to which they bind. The goal was to understand the number of epitopes (linear and/or conformational) on an antigen that is recognized by monoclonal IgE antibodies (expressed as IgG4s), and to understand the distribution of the monoclonal antibody epitope specificity (i.e. is there a bias towards immunodominant epitopes?). The antibodies were then grouped by epitope bin, which, when combined with affinity data, enables the informed selection of monoclonal antibodies to be further evaluated in functional assays like a blocking ELISA, basophil activation test (BAT), or mast cell activation test (MAT).

For measurement of kinetic parameters, antibodies were first coupled to HC30M sensor chips, and the sensor chips were then activated. Antibodies were coupled to the activated surface at multiple concentrations, with remaining reactive groups subsequently inactivated. Natural Ara h 2 or Ara h 6 (InBio) was serially injected at multiple concentrations using the Carterra LSA single flow cell over the antibody-coupled chip without regeneration between injections. Association and dissociation curves were fit using a 1:1 Langmuir binding model to obtain kd, ka, and KD with Kinetics software (Carterra).

For epitope binning, surfaces were functionalized with antibodies as described above. Each binding cycle consisted of natural Ara h 2 or Ara h 6 injected using the Carterra LSA single flow cell, followed by antibody, and then regeneration. Analysis of epitope binning was performed by Epitope software (Carterra). Binding of antibody was normalized to signal from the buffer-only injections that follow antigen injections. Antibody pairs were classified as blocking or sandwiching according to whether they met binding signal thresholds above the buffer injection background.

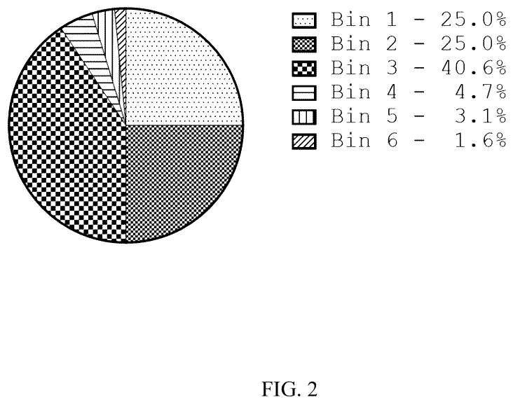

Using SPR, it was determined that over 90% of 64 Ara h 2-specific mAbs were high-affinity in the sub-nanomolar range. Additionally, it was found that 90% of these mAbs belonged to epitope bins 1, 2, or 3 (FIG. 2). This led to the selection and engineering of mAbs that belonged to these three epitope bins for in vitro characterization. FIG. 2 illustrates the distribution of 64 mAbs by Ara h 2 epitope bin and highlights three immunodominant epitope bins.

### Example 3. In Vitro Characterization by ELISA

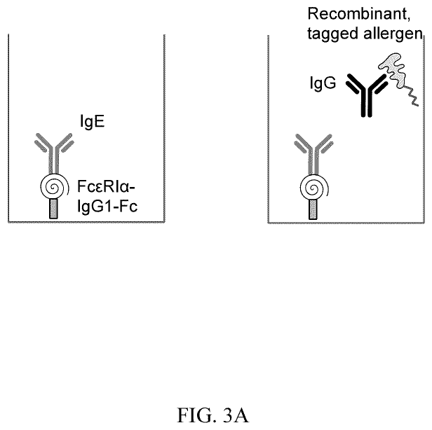

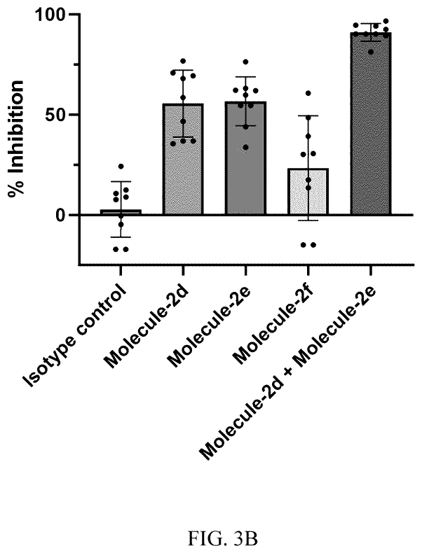

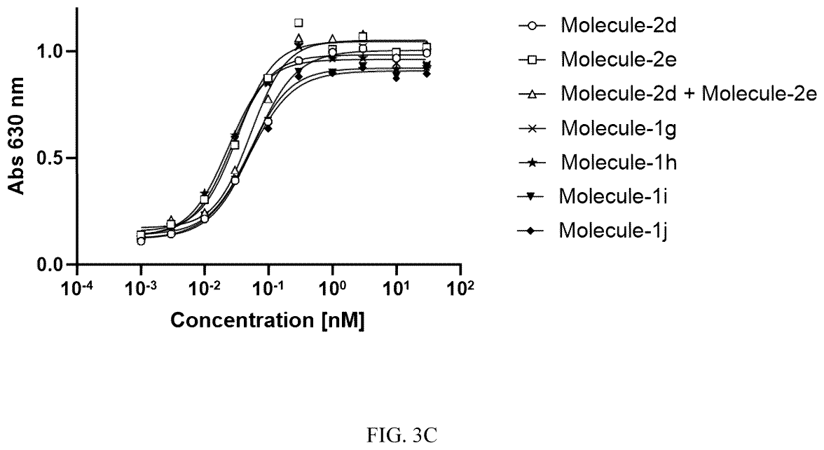

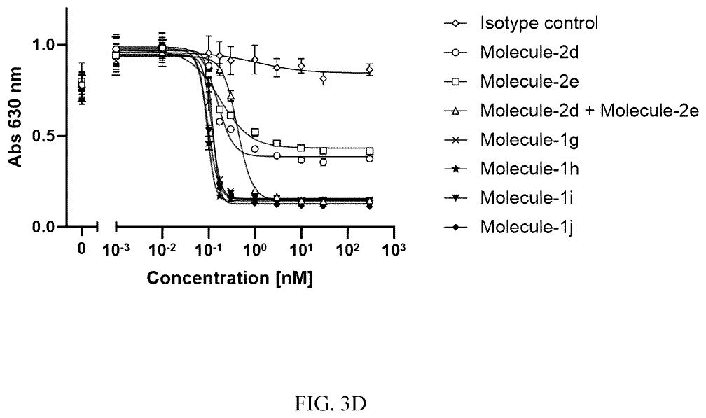

In vitro ELISAs were used to characterize the binding and inhibitory capacities of IgG mAbs. The in vitro blocking ELISA was used to assess the ability of one or more IgG monoclonal antibodies to block the binding of allergic plasma IgE to recombinant Ara h 2. In this assay, a simplified version of which is shown in FIG. 3A, human FcεRIα-IgG Fc fusion protein captures human IgE from peanut allergic plasma. Following this capture, a preincubated mixture of His-tagged recombinant Ara h 2 and a titrated series of antibody is added. An anti-His HRP-conjugated detection antibody and TMB were used to measure recombinant Ara h 2 bound by plasma IgE. Inhibition was calculated relative to no-antibody control wells.

It was found that independently, Molecule-2d, Molecule-2e, and Molecule-2f inhibit binding of allergic plasma IgE to recombinant Ara h 2 to variable degrees (FIG. 3B), whereas the combination of Molecule-2d and Molecule-2e had greater and more consistent inhibition, achieving 91% average inhibition across 9 peanut allergic plasmas. Molecule-2d, Molecule-2e, and Molecule-2fare high affinity IgG4 antibodies with the S228P hinge-stabilizing mutation belonging to epitope bins 1, 2, and 3, respectively.

TABLE 5
Molecule-2 Naming Reference

| | | CMC | | | |
|---|---|---|---|---|---|
| | | liabilities | | SEQ ID | SEQ ID |
| Name | Binder- | removed | Constant region | NO: HC | NO: LC |
| Molecule-2a | 1 | Y | IgG4 S228P/no-CtermK | 15 | 17 |
| Molecule-2b | 2 | Y | IgG4 S228P/no-CtermK | 32 | 34 |
| Molecule-2c | 3 | Y | IgG4 S228P/no-CtermK | 48 | 50 |
| Molecule-2d | 1 | N | IgG4 S228P | 72 | 17 |
| Molecule-2e | 2 | N | IgG4 S228P | 74 | 76 |
| Molecule-2f | 3 | N | IgG4 S228P | 77 | 50 |
| Molecule-2g | 1 | N | IgG1 | 73 | 17 |
| Molecule-2h | 2 | N | IgG1 | 75 | 76 |
| Molecule-2i | 3 | N | IgG1 | 78 | 50 |

Subsequently, a number of human IgG4-scFv antibodies, named with a “Molecule-1” prefix, were engineered based on the combination of binding domains, optionally further modified to remove chemistry, manufacturing, and controls (CMC) liabilities, taken from Molecule-2d, Molecule-2e, and Molecule-2f. These engineered molecules are bispecific, but more specifically, have bi-epitopic specificity. These bi-epitopic antibodies bind through Fab domains to one Ara h 2 epitope, either “bin 1”, or “bin 2”, or “bin 3”, and through scFv domains to a different epitope chosen from the same set. The bin 1 and bin 2 epitopes are conserved in the homologous peanut protein Ara h 6, while the “bin 3” epitope is unique to Ara h 2.

TABLE 6
Molecule-1 Naming Reference

| | | scFv (Binder-), in | | | |
|---|---|---|---|---|---|
| | | VH then VL order | CMC | SEQ | SEQ |
| | Fab | unless stated | liabilities | ID NO: | ID NO: |
| Name | (Binder-) | otherwise | removed | HC | LC |
| Molecule-1a | 2 | 1 | Y | 53 | 52 |
| Molecule-1b | 1 | 2 | Y | 55 | 54 |
| Molecule-1c | 1 | 3 | Y | 57 | 56 |
| Molecule-1d | 3 | 1 | Y | 59 | 58 |
| Molecule-1e | 2 | 3 | Y | 61 | 60 |
| Molecule-1f | 3 | 2 | Y | 63 | 62 |
| Molecule-1g | 2 | 1 | N | 65 | 64 |
| Molecule-1h | 2 | 1 (VL then VH) | N | 67 | 66 |
| Molecule-1i | 1 | 2 | N | 69 | 68 |
| Molecule-1j | 1 | 2 (VL then VH) | N | 71 | 70 |

Four Molecule-1 mAbs were evaluated for their ability to bind to recombinant Ara h 2. All were found to bind in a concentration-dependent fashion similar to one another and similar to Molecule-2d, Molecule-2e, and the combination thereof (FIG. 3C). Next, the ability of these molecules to inhibit recombinant Ara h 2 from binding polyclonal peanut allergic plasma IgE was evaluated. All four Molecule-1 mAbs exhibited greater inhibition than either Molecule-2d or Molecule-2e alone and inhibited at a lower IC50 than the combination of Molecule-2d and Molecule-2e (FIG. 3D).

FIG. 3A illustrates a simplified ELISA for assessing the ability of IgG mAbs to block the binding of allergic plasma IgE to a tagged allergen. FIG. 3B illustrates inhibition of recombinant Ara h 2 binding to peanut allergic plasma IgE by one or more Molecule-2 mAbs in the blocking ELISA. Isotype control=F080-F1, an IgG4 mAb specific to a non-peanut allergen. Data show individual peanut allergic plasmas (n=9) with bars indicating mean±SD. FIG. 3C illustrates binding of Molecule-1 and Molecule-2 mAbs to recombinant Ara h 2. FIG. 3D illustrates Molecule-1 mAbs inhibiting peanut allergic plasma IgE from binding recombinant Ara h 2 relative to Molecule-2 mAbs alone or in combination for one peanut allergic plasma. Isotype control=F080-F1, an IgG4 mAb specific to a non-peanut allergen.

### Example 4. In Vitro Characterization by Mast Cell Activation Tests (MATs)

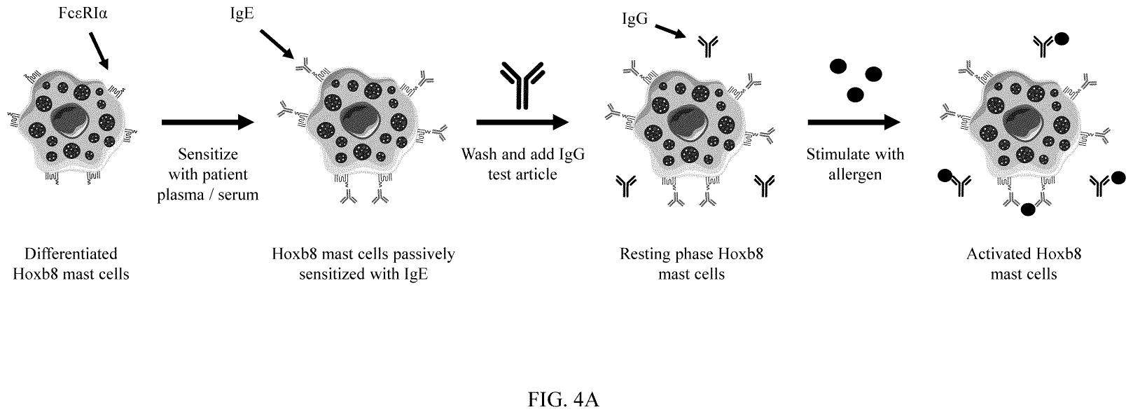

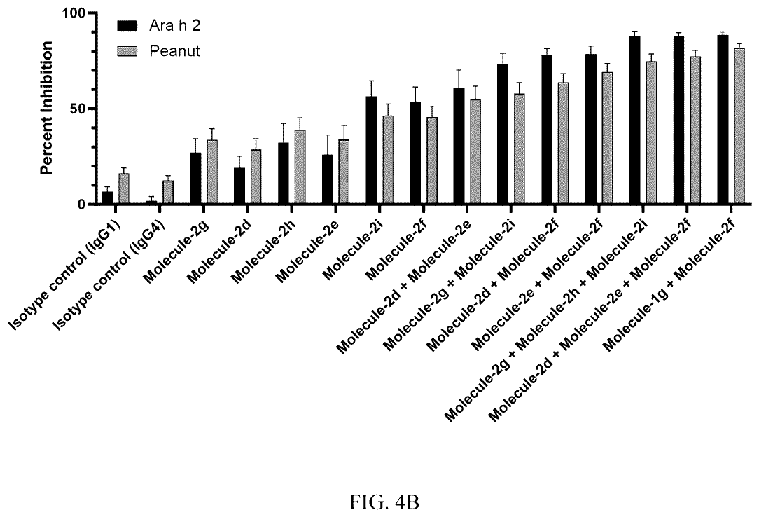

An overview of the MAT experimental design is illustrated in FIG. 4A. Murine Hoxb8 mast cells expressing human FcεRIα were first sensitized with patient plasma, which allowed them to capture polyclonal IgE via the high affinity FCεRI. Subsequently, cells were washed, incubated with IgG4 antibody for 30 min, and then, without washing, a predetermined, optimal concentration of Ara h 2 or peanut protein was added. This optimal concentration of Ara h 2 or peanut protein necessary for slightly above half-maximal activation was predetermined for each plasma separately. Activation was assessed by CD107a expression using flow cytometry and percent inhibition was calculated relative to activation of no antibody control wells.

mAbs with both human IgG1 and human IgG4 S228P constant regions were evaluated in the MAT using seven peanut allergic plasmas. The constant region was found to have little effect when comparing otherwise identical Molecule-2 mAbs, e.g. Molecule-2g and Molecule-2d, Molecule-2h and Molecule-2e, and Molecule-2i and Molecule-2f (FIG. 4B). As compared to individual Molecule-2 mAbs, combinations of two or three had improved levels of inhibition, with the greatest inhibition of peanut-mediated mast cell activation achieved by a combination of Molecule-2f (IgG4 S228P) and bi-epitopic Molecule-Ig (IgG4 S228P).

FIG. 4A illustrates an overview of mast cell activation test (MAT). FIG. 4B illustrates percent inhibition of peanut-mediated and Ara h 2-mediated mast cell activation by human IgG1 and human IgG4 S228P mAbs. The total antibody concentration is 10 μg/mL for all test articles. Isotype control (IgG1 and IgG4 S228P)=G010-F5, a mAb specific to a non-peanut allergen. Mean±SEM of n=7 peanut allergic plasmas shown.

### Example 5. In Vitro Characterization of Combination Therapy

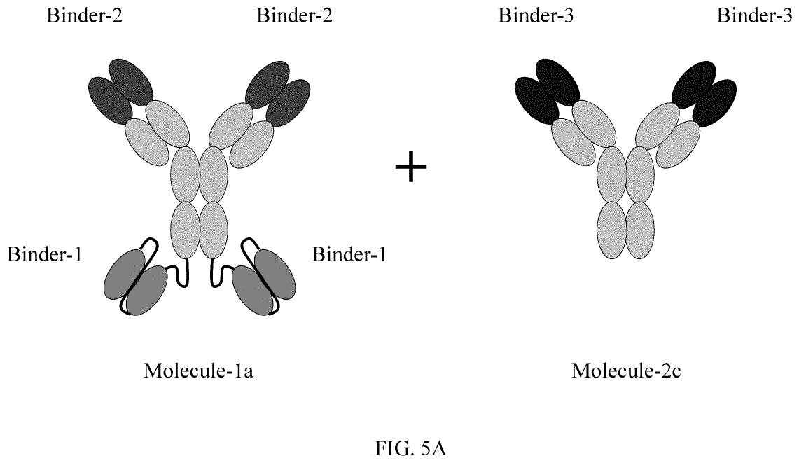

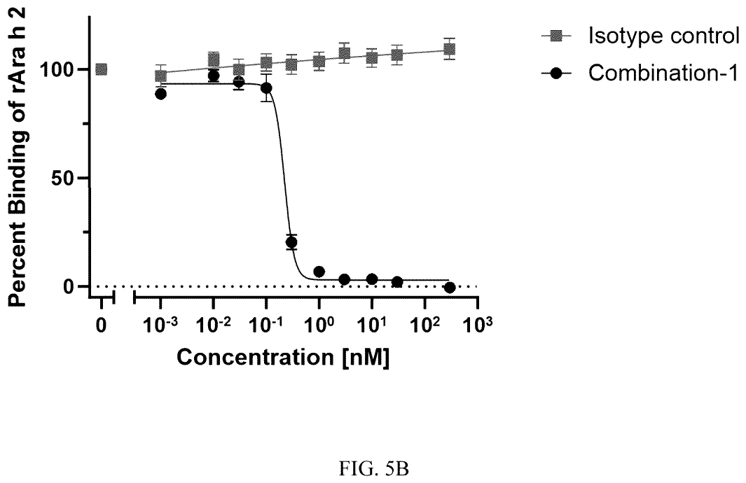

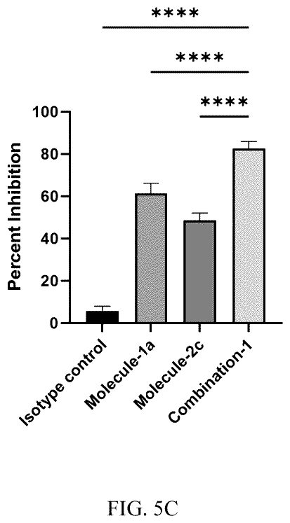

Based upon the experiments in the preceding examples, a combination therapy was developed (Combination-1). Combination-1 consists of a 1:1 by mass mixture of Molecule-1a and Molecule-2c as shown in FIG. 5A. Molecule-1a is an IgG4 antibody with a stabilized hinge region (S228P) in which an scFv domain is fused to the c-terminus of each heavy chain. Binder-1 of Molecule-1 binds to epitope bin 1 on Ara h 2 and Ara h 6. Binder-2 of Molecule-1 binds to epitope bin 2 on Ara h 2 and Ara h 6. The binding is bi-valent for both epitopes. Molecule-2 is an IgG4 S228P antibody that lacks the heavy chain constant region c-terminal lysine. Binder-3 binds to epitope bin 3 present on Ara h 2 only. The binding is bi-valent for this epitope.

Monoclonal antibodies were produced by transient transfection of mammalian cells. Heavy and light chain variable region genes from each mAb were synthesized after codon optimization, cloned into pcDNA3.4 vectors containing the appropriate constant region (IgG1 or IgG4 S228P, and either A or k, as appropriate), and transiently co-transfected into Chinese hamster ovary (CHO) cells. After approximately 7 days in culture, antibodies were purified from the supernatant with 1-step protein A purification. After washing and elution, the eluted fractions were pooled and buffer exchanged to a suitable formulation buffer. Antibodies were characterized by, at minimum, SDS-PAGE under reducing and nonreducing conditions.

For larger scale monoclonal antibody production, stable cell lines were generated using a suitable retroviral system. A characterized CHO cell line was transfected with cDNA corresponding to the heavy and light chains and stable cell lines were subsequently selected following single-cell subcloning and characterization of clones in shake flasks and small-scale bioreactors. To produce each mAb, CHO cells were grown in bioreactors, and mAb secreted into the media was recovered by conventional IgG-purification techniques.

In order to evaluate the potency and activity of Combination-1, a number of experiments were conducted. Using the blocking ELISA format described in Example 3, Combination-1 completely inhibited recombinant Ara h 2 binding to IgE across five peanut allergic plasmas, whereas no inhibition was observed for the isotype control (FIG. 5B). Next, the MAT described in Example 4 was used to evaluate Combination-1 relative to its constituent components Molecule-1a and Molecule-2c. Compared to the isotype control, Combination-1 significantly inhibited peanut-mediated mast cell activation in 19 peanut allergic plasmas (FIG. 5C). Average inhibition was 82.6% with a maximal inhibition of 97.5% for Combination-1 compared to an average inhibition of 5.8% for the isotype control (IgG4 trastuzumab). Average inhibition was 61.4% for Molecule-1a alone and 48.7% for Molecule-2c alone, both significantly less than Combination-1. The coefficient of variation was also greater for Molecule-1a alone and Molecule-2c alone compared to Combination-1.

FIG. 5A illustrates Combination-1 comprising two molecules. FIG. 5B illustrates blocking of rAra h 2 binding to peanut allergic plasma IgE by Combination-1. Isotype control=F080-F1, an IgG4 mAb specific to a non-peanut allergen. Mean±SEM of n=5 plasmas shown. FIG. 5C illustrates inhibition of peanut-mediated mast cell activation by Combination-1 and its constituent components for n=19 peanut allergic plasmas. The total antibody concentration is 10 μg/mL for all test articles. Isotype control=IgG4 trastuzumab. Mean±SEM shown. ****P<0.001 by one-way ANOVA followed by Bonferroni's multiple comparisons test.

### Example 6. In Vivo Characterization of Combination Therapy in a Mouse Model of Peanut Allergy

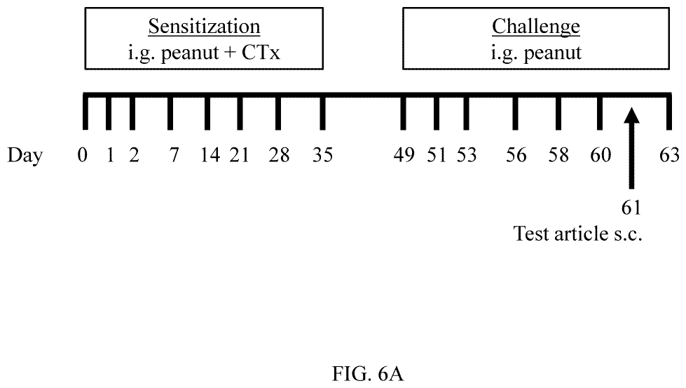

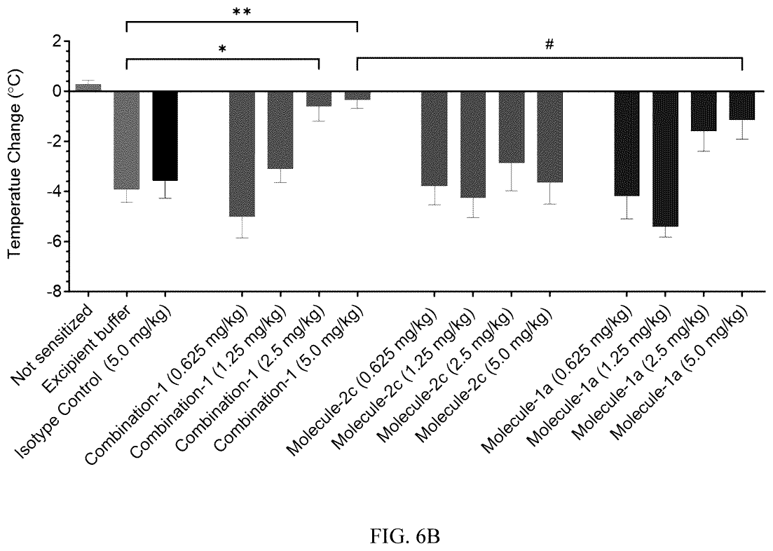

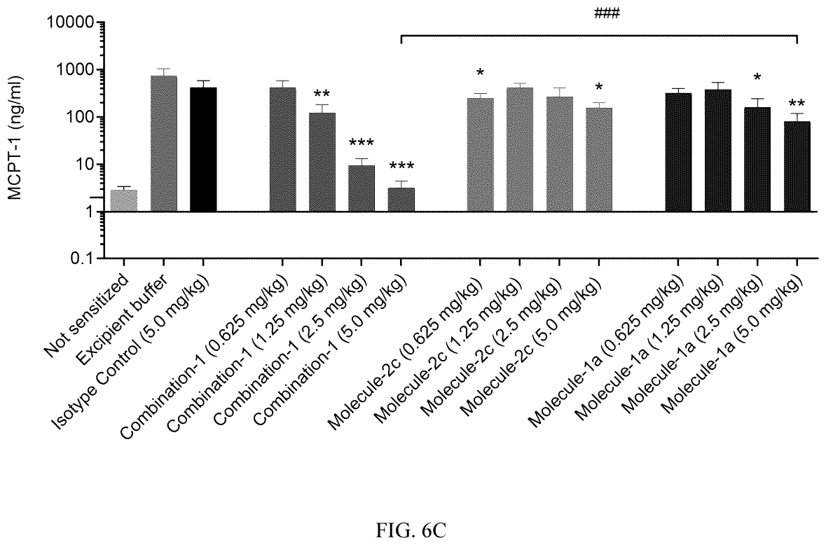

The efficacy of Combination-1 was evaluated in a mouse model of peanut allergy based on an adapted protocol from Landers et al. Targeted allergen-specific immunotherapy within the skin improves allergen delivery to induce desensitization to peanut. Immunotherapy. 2022 May; 14(7):539-552. doi: 10.2217/imt-2021-0206. Epub 2022 Feb. 24. Briefly, naive C3H/HeJ female mice were sensitized on Weeks 0-5 with 2 mg peanut protein+10 μg cholera toxin (CTx) delivered via oral gavage. A peanut challenge protocol was initiated on day 49. The challenge protocol consisted of 7 challenges performed by oral gavage with 25 mg peanut protein on alternating days over a 2-week period. At day 61, 48 hours prior to the 7th peanut protein challenge, mice were treated with either test articles or excipient buffer (vehicle) by subcutaneous injection (FIG. 6A). Reactions to the 7′ peanut protein challenge, which was performed 48 hours after administration of the test articles, were recorded. Mice were monitored for core body temperature and observers were blinded to treatment. Sixty minutes after the final peanut protein challenge, mice were euthanized and blood was collected by cardiac puncture. Serum was collected and analyzed for levels of human IgG and mast cell protease 1 (MCPT-1), an indicator of mast cell degranulation.

Unless stated otherwise, all data were analyzed using a one-way ANOVA followed by a Dunnett's multiple comparisons test to compare each treatment group to the excipient buffer treated group. Statistical significance is noted as *P<0.05; **P<0.01 and ***P<0.001 and values are reported as mean±SEM.

In this study, forty-eight hours prior to the 7′ challenge animals were dosed with either test article (Combination-1, Molecule-1a, Molecule-2c at doses of 0.625, 1.25, 2.5 or 5.0 mg/kg), isotype control (IgG4 trastuzumab at 5.0 mg/kg) or excipient buffer. The 7th peanut protein challenge resulted in a severe anaphylactic response as measured by a 3.9±0.5° C. fall in rectal temperature in animals that were treated with excipient buffer (FIG. 6A). This hypothermic response to peanut challenge was dose-dependently reversed in the Combination-I-treated animals and the reduced response in the Combination-1, 2.5 and 5.0 mg/kg-treated groups was statistically significant compared to that measured in the excipient buffer-treated group. In animals pre-treated with Molecule-1a alone there was a dose-dependent reduction in the hypothermic response to peanut challenge; however, the responses at all dose levels were not statistically different from the response seen in the excipient buffer-treated group. A direct comparison (unpaired Student's t-test) between the highest dose of the Combination-1 and Molecule-1a showed that there was a significant difference between the two treatments. Molecule-2c alone resulted in no significant reduction in the hypothermic response to peanut protein challenge at any of the doses tested. The isotype control antibody (IgG4 trastuzumab) had no effect on the hypothermic response.

At the end of the 60 min post-challenge observation period the animals were euthanized, and a terminal blood sample collected. A fraction of this sample was assayed for MCPT-1 as a direct measure of mast cell degranulation. Combination-1 reduced MCPT-1 relative to the excipient buffer treated animals in a dose-dependent fashion, achieving levels equivalent to those seen in the non-sensitized animals at 5 mg/kg. Molecule-1a alone yielded a marked reduction in MCPT-1 serum levels but this was significantly less than that associated with Combination-1 at comparable doses (5.0 mg/kg). Neither the isotype control antibody (IgG4 trastuzumab) nor Molecule-2c meaningfully decreased MCPT-1 levels.

FIG. 6A illustrates a protocol for a mouse model of peanut allergy oral sensitization/oral peanut challenge. At day 61, 48 hours prior to the 7th peanut protein challenge, mice were treated with either test articles or excipient buffer (vehicle) by subcutaneous (s.c.) injection. Sixty minutes after the final peanut protein challenge on day 63, mice were euthanized and blood was collected by cardiac puncture. Serum was collected and analyzed for levels of human IgG and MCPT-1. FIG. 6B illustrates the dose-dependent effects of Combination-1 and its constituent components on the hypothermic response in peanut allergic animals when challenged with 25 mg of peanut protein delivered via oral gavage. Isotype control=IgG4 trastuzumab. All data were compared to the effects of excipient buffer. The effects of Combination-1 and Molecule-1a (5.0 mg/kg) were compared using an unpaired Student's t-test (#P<0.05). FIG. 6C illustrates the dose-dependent effects of Combination-1, Molecule-1a and Molecule-2c on serum MCPT-1 as a direct measure of mast cell degranulation in peanut allergic animals when challenged with 25 mg of peanut protein delivered via oral gavage. Isotype control=IgG4 trastuzumab. All data are compared to the effects of excipient buffer. The effects of Combination-1 and Molecule-1a (5.0 mg/kg) were compared using an unpaired Student's t-test (###). Note that serum levels of MCPT-1 in animals treated with 5 mg/kg Combination-1 were equivalent to those in non-sensitized animals.

What is claimed is:

1. A composition that comprises a first binding domain that binds to Ara h 2 and Ara h 6 wherein the first binding domain comprises a heavy chain variable domain (VH) that comprises complementarity determining regions (CDRs): HC-CDR1, HC-CDR2, HC-CDR3, wherein the HC-CDR1, HC-CDR2, HC-CDR3, comprise the amino acid sequences according to HC-CDR1: SEQ ID NO: 1, HC-CDR2: SEQ ID NO: 2 or SEQ ID NO: 3, HC-CDR-3: SEQ ID NO: 4 or SEQ ID NO: 5, and the first binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 6, LC-CDR2: SEQ ID NO: 7, LC-CDR-3: SEQ ID NO: 8, wherein the composition comprises a second binding domain that binds to Ara h 2 and Ara h 6 wherein the second binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 18, HC-CDR2: SEQ ID NO: 19, HC-CDR-3: SEQ ID NO: 20 or SEQ ID NO: 21, and the second binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 22, LC-CDR2: SEQ ID NO: 23, LC-CDR-3: SEQ ID NO: 24, and wherein the composition comprises a third binding domain that binds to Ara h 2 wherein the third binding domain comprises a VH that comprises CDRs: HC-CDR1, HC-CDR2, HC-CDR3, wherein the HC-CDR1, HC-CDR2, HC-CDR3, comprise the amino acid sequences according to HC-CDR1: SEQ ID NO: 35, HC-CDR2: SEQ ID NO: 36 or SEQ ID NO: 37, HC-CDR-3: SEQ ID NO: 38, and the third binding domain comprises a light chain variable (VL) domain that comprises CDRs: LC-CDR1, LC-CDR2, LC-CDR3, wherein the LC-CDR1, LC-CDR2, LC-CDR3, comprise the amino acid sequences according to LC-CDR1: SEQ ID NO: 39, LC-CDR2: SEQ ID NO: 40, LC-CDR-3: SEQ ID NO: 41.

2. The composition of claim 1, wherein the VH of the first binding domain comprises an amino acid sequence with at least 80%, 85%, 90%, 95%, 98%, 99%, or 100% sequence identity to SEQ ID NO: 9 or SEQ ID NO: 10.

3. The composition of claim 1, wherein the VL of the first binding domain comprises an amino acid sequence with at least 80%, 85%, 90%, 95%, 98%, 99%, or 100% sequence identity to SEQ ID NO: 11.

4. The composition of claim 1, wherein the VH of the first binding domain comprises an amino acid sequence with at least 80%, 85%, 90%, 95%, 98%, 99%, or 100% sequence identity to SEQ ID NO: 9 or SEQ ID NO: 10 and wherein the VL of the first binding domain comprises an amino acid sequence with at least 80%, 85%, 90%, 95%, 98%, 99%, or 100% sequence identity to SEQ ID NO: 11.

5. The composition of claim 1, wherein the first binding domain comprises a Fab, Fab′, F(ab′)₂, or a single chain variable fragment (scFv).

6. The composition of claim 1, wherein the first binding domain is a Fab, Fab′, F(ab′)₂, or a single chain variable fragment (scFv).

7. The composition of claim 5, wherein the first binding domain comprises the scFv.

8. The composition of claim 1, wherein the VH of the first binding domain and VL of the first binding domain are connected by an scFv linker sequence.

9. The composition of claim 8, wherein the scFv linker sequence of the first binding domain comprises an amino acid sequence according to SEQ ID No: 12.

10. The composition of claim 7, wherein the scFv of the first binding domain comprises an amino acid sequence with at least 80%, 85%, 90%, 95%, 98%, 99%, or 100% sequence identity to SEQ ID NO: 13 or SEQ ID NO: 14.

11. The composition of claim 1, wherein the first binding domain comprises a constant domain.

12. The composition of claim 11, wherein the constant domain of the first binding domain comprises a human IgG4 sequence.

13. The composition of claim 12, wherein the human IgG4 sequence of the first binding domain comprises a S228P point mutation wherein the amino acid numbering is according to the EU index in Kabat et al.

14. The composition of claim 12, wherein the human IgG4 sequence of the first binding domain does not have a C-terminal lysine residue.

15. The composition of claim 11, wherein the constant domain of the first binding domain comprises a human IgG1 sequence.

16. The composition of claim 11, wherein the constant domain of the first binding domain comprises a C_{H}1, C_{H}2, C_{H}3, or a C_{L} amino acid sequence.

17. The composition of claim 11, wherein the first binding domain comprises a C_{H}1, C_{H}2, C_{H}3, and a C_{L} amino acid sequence.

18. The composition of claim 1, wherein the first binding domain comprises an amino acid sequence with at least 80%, 85%, 90%, 95%, 98%, 99%, or 100% sequence identity to SEQ ID NO:15, SEQ ID NO: 16, SEQ ID NO: 72, or SEQ ID NO: 73, and the first binding domain comprises an amino acid sequence with at least 80%, 85%, 90%, 95%, 98%, 99%, or 100% sequence identity to SEQ ID NO: 17.

19. The composition of claim 1, wherein the VH of the second binding domain comprises an amino acid sequence with at least 80%, 85%, 90%, 95%, 98%, 99%, or 100% sequence identity to SEQ ID NO: 25 or SEQ ID NO: 26.

20. The composition of claim 1, wherein the VL of the second binding domain comprises an amino acid sequence with at least 80%, 85%, 90%, 95%, 98%, 99%, or 100% sequence identity to SEQ ID NO: 27 or SEQ ID NO: 28.

21. The composition of claim 1, wherein the VH of the second binding domain comprises an amino acid sequence with at least 80%, 85%, 90%, 95%, 98%, 99%, or 100% sequence identity to SEQ ID NO: 25 or SEQ ID NO: 26 and wherein the VL of the second binding domain comprises an amino acid sequence with at least 80%, 85%, 90%, 95%, 98%, 99%, or 100% sequence identity to SEQ ID NO: 27 or SEQ ID NO: 28.

22. The composition of claim 1, wherein the second binding domain comprises a Fab, Fab′, F(ab′)₂, or a single chain variable fragment (scFv).

23. The composition of claim 1, wherein the second binding domain is a Fab, Fab′, F(ab′)₂, or a single chain variable fragment (scFv).

24. The composition of claim 22, wherein the second binding domain comprises the scFv.

25. The composition of claim 24, wherein the VH of the second binding domain and VL of the second binding domain are connected by an scFv linker sequence.

26. The composition of claim 25, wherein the scFv linker sequence of the second binding domain comprises an amino acid sequence according to SEQ ID NO: 29.

27. The composition of claim 22, wherein the scFv of the second binding domain comprises an amino acid sequence with at least 80%, 85%, 90%, 95%, 98%, 99%, or 100% sequence identity to SEQ ID NO: 30 or SEQ ID NO: 31.

28. The composition of claim 1, wherein the second binding domain comprises a constant domain.

29. The composition of claim 28, wherein the constant domain of the second binding domain comprises a human IgG4 sequence.

30. The composition of claim 29, wherein the human IgG4 sequence of the second binding domain comprises a S228P point mutation wherein the amino acid numbering is according to the EU index in Kabat et al.

31. The composition of claim 29, wherein the human IgG4 sequence of the second binding domain does not have a C-terminal lysine residue.

32. The composition of claim 28, wherein the constant domain of the second binding domain comprises a human IgG1 sequence.

33. The composition of claim 28, wherein the constant domain of the second binding domain comprises a C_{H}1, C_{H}2, C_{H}3, or a C_{L} amino acid sequence.

34. The composition of claim 28, wherein the second binding domain comprises a C_{H}1, C_{H}2, C_{H}3, and a C_{L} amino acid sequence.

35. The composition of claim 1, wherein the second binding domain comprises an amino acid sequence with at least 80%, 85%, 90%, 95%, 98%, 99%, or 100% sequence identity to SEQ ID NO: 32, SEQ ID NO: 33, SEQ ID NO: 74, or SEQ ID NO: 75 and the isolated antibody comprises an amino acid sequence with at least 80%, 85%, 90%, 95%, 98%, 99%, or 100% sequence identity to SEQ ID NO: 34 or SEQ ID NO: 76.

36. The composition of claim 1, wherein the VH of the third binding domain comprises an amino acid sequence with at least 80%, 85%, 90%, 95%, 98%, 99%, or 100% sequence identity to SEQ ID NO: 42 or SEQ ID NO: 43.

37. The composition of claim 1, wherein the VL of the third binding domain comprises an amino acid sequence with at least 80%, 85%, 90%, 95%, 98%, 99%, or 100% sequence identity to SEQ ID NO: 44.

38. The composition of claim 1, wherein the VH of the third binding domain comprises an amino acid sequence with at least 80%, 85%, 90%, 95%, 98%, 99%, or 100% sequence identity to SEQ ID NO: 42 or SEQ ID NO: 43 and wherein the VL of the third binding domain comprises an amino acid sequence with at least 80%, 85%, 90%, 95%, 98%, 99%, or 100% sequence identity to SEQ ID NO: 44.

39. The composition of claim 1, wherein the third binding domain comprises a Fab, Fab′, F(ab′)₂, or a single chain variable fragment (scFv).

40. The composition of claim 1, wherein the third binding domain is a Fab, Fab′, F(ab′)₂, or a single chain variable fragment (scFv).

41. The composition of claim 39, wherein the third binding domain comprises the scFv.

42. The composition of claim 39, wherein the VH of the third binding domain and VL of the third binding domain are connected by an scFv linker sequence.

43. The composition of claim 42, wherein the scFv linker sequence of the third binding domain comprises an amino acid sequence according to SEQ ID NO: 45.

44. The composition of claim 39, wherein the scFv of the third binding domain comprises an amino acid sequence with at least 80%, 85%, 90%, 95%, 98%, 99%, or 100% sequence identity to SEQ ID NO: 46 or SEQ ID NO: 47.

45. The composition of claim 1, wherein the third binding domain comprises a constant domain.

46. The composition of claim 45, wherein the constant domain of the third binding domain comprises a human IgG4 sequence.

47. The composition of claim 46, wherein the human IgG4 sequence of the third binding domain comprises a S228P point mutation wherein the amino acid numbering is according to the EU index in Kabat et al.

48. The composition of claim 46, wherein the human IgG4 sequence of the third binding domain does not have a C-terminal lysine residue.

49. The composition of claim 45, wherein the constant domain of the third binding domain comprises a human IgG1 sequence.

50. The composition of claim 45, wherein the constant domain of the third binding domain comprises a C_{H}1, C_{H}2, C_{H}3, or a C_{L} amino acid sequence.

51. The composition of claim 45, wherein the third binding domain comprises a C_{H}1, C_{H}2, C_{H}3, and a C_{L} amino acid sequence.

52. The composition of claim 1, wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the isolated antibody comprises an amino acid sequence with at least 80%, 85%, 90%, 95%, 98%, 99%, or 100% sequence identity to SEQ ID NO: 50.

53. The composition of claim 1, wherein the first binding domain, second binding domain, and the third binding domain are connected within the same molecule.

54. The composition of claim 1, wherein the first binding domain, second binding domain, and the third binding domain are separate molecules.

55. The composition of claim 1, wherein the first binding domain and second binding domain are within the same molecule and the third binding domain is a separate molecule from the molecule with the first binding domain and the second binding domain.

56. The composition of claim 1, wherein the first binding domain and third binding domain are within the same molecule and the second binding domain is a separate molecule from the molecule with the first binding domain and the third binding domain.

57. The composition of claim 1, wherein the second binding domain and third binding domain are within the same molecule and the first binding domain is a separate molecule from the molecule with the second binding domain and the third binding domain.

58. The composition of claim 1, wherein the first binding domain comprises a scFv and the second binding domain comprises a C_{H}1, C_{H}2, C_{H}3, and a C_{L} amino acid sequence.

59. The composition of claim 1, wherein the second binding domain comprises a scFv and the first binding domain comprises a C_{H}1, C_{H}2, C_{H}3, and a C_{L} amino acid sequence.

60. The composition of claim 1, wherein the first binding domain and the second binding domain are connected by a linking moiety.

61. The composition of claim 1, wherein the linking moiety is an amino acid sequence.

62. The composition of claim 1, wherein the linking moiety is at least 4 amino acids long.

63. The composition of claim 1, wherein the linking moiety is between 4-20 amino acids in length.

64. The composition of claim 1, wherein the linking moiety connects the C_{H}3 amino acid sequence of the second binding domain to the scFv of the first binding domain.

65. The composition of claim 1, wherein the linking moiety comprises the amino acid sequence of SEQ ID NO: 51.

66. The composition of claim 1, wherein the first and second binding domains comprise an amino acid sequence with at least 80%, 85%, 90%, 95%, 98%, 99%, or 100% sequence identity to SEQ ID NO: 53 and an amino acid sequence with at least 80%, 85%, 90%, 95%, 98%, 99%, or 100% sequence identity to SEQ ID NO: 52 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

67. The composition of claim 1, wherein the first and second binding domains comprise an amino acid sequence with at least 80%, 85%, 90%, 95%, 98%, 99%, or 100% sequence identity to SEQ ID NO: 55 and an amino acid sequence with at least 80%, 85%, 90%, 95%, 98%, 99%, or 100% sequence identity to SEQ ID NO: 54 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

68. The composition of claim 1, wherein the first and second binding domains comprise an amino acid sequence with at least 80%, 85%, 90%, 95%, 98%, 99%, or 100% sequence identity to SEQ ID NO: 65 and an amino acid sequence with at least 80%, 85%, 90%, 95%, 98%, 99%, or 100% sequence identity to SEQ ID NO: 64 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

69. The composition of claim 1, wherein the first and second binding domains comprise an amino acid sequence with at least 80%, 85%, 90%, 95%, 98%, 99%, or 100% sequence identity to SEQ ID NO: 67 and an amino acid sequence with at least 80%, 85%, 90%, 95%, 98%, 99%, or 100% sequence identity to SEQ ID NO: 66 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

70. The composition of claim 1, wherein the first and second binding domains comprise an amino acid sequence with at least 80%, 85%, 90%, 95%, 98%, 99%, or 100% sequence identity to SEQ ID NO: 69 and an amino acid sequence with at least 80%, 85%, 90%, 95%, 98%, 99%, or 100% sequence identity to SEQ ID NO: 68 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

71. The composition of claim 1, wherein the first and second binding domains comprise an amino acid sequence with at least 80%, 85%, 90%, 95%, 98%, 99%, or 100% sequence identity to SEQ ID NO: 71 and an amino acid sequence with at least 80%, 85%, 90%, 95%, 98%, 99%, or 100% sequence identity to SEQ ID NO: 70 and wherein the third binding domain comprises an amino acid sequence with at least 80%, 85%, 90%, 95%, 98%, 99%, or 100% sequence identity to SEQ ID NO: 48, SEQ ID NO: 49, SEQ ID NO: 77, or SEQ ID NO: 78 and the third binding domain comprises an amino acid sequence with at least 80%, 85%, 90%, 95%, 98%, 99%, or 100% sequence identity to SEQ ID NO: 50.

72. The composition of claim 1, wherein the composition comprises a pharmaceutically acceptable excipient.

73. An isolated recombinant nucleic acid sequence encoding an amino acid sequence of the composition according to any one of claims 1-72.

74. A vector comprising the isolated recombinant nucleic acid sequence according to claim 73.

75. A kit that comprises at least one of:
a. the composition of any one of claims 1-72;
b. the vector of claim 74; or
c. the nucleic acid molecule of claim 73.

76. A method of treating a food allergy in a subject in need thereof comprising administering to the subject the composition according to any one of claims 1-72.

77. The method of claim 76, wherein the food allergy comprises a peanut allergy.
